(19) United States
(12) Patent Application Publication
(10) Pub. No.: US 2009/0170775 A1
(43) Pub. Date: Jul. 2, 2009

(54) VASOACTIVE INTESTINAL POLYPEPTIDE COMPOSITIONS

(75) Inventor: John J. Nestor, JR., Encinitas, CA (US)

Correspondence Address: HOWSON & HOWSON LLP, 501 OFFICE CENTER DRIVE, SUITE 210, FORT WASHINGTON, PA, 19034, US

(73) Assignee: Transition Therapeutics, Inc., Toronto (CA)

(21) Appl. No.: 12/370,842

(22) Filed: Feb. 13, 2009

Related U.S. Application Data
(63) Continuation of application No. 12/093,195.
Application No. 12/093,195 is a continuation-in-part of application No. 11/279,238, filed on Apr. 10, 2006.
Application No. 11/279,238 is a continuation-in-part of application No. 11/245,499, filed on Oct. 7, 2005.
Application No. 11/245,499 is a continuation-in-part of application No. 11/869,032, filed on Oct. 9, 2007.
Application No. 11/869,032 is a continuation-in-part of application No. 11/539,613, filed on Oct. 6, 2006.
Application No. 11/539,613 is a continuation-in-part of application No. 11/279,238, filed on Apr. 10, 2006.
Application No. 11/279,238 is a continuation-in-part of application No. 11/245,499, filed on Oct. 7, 2005.
Application No. 11/245,499 is a continuation-in-part of application No. PCT/US2007/080738, filed on Oct. 8, 2007.
Application No. PCT/US2007/080738 is a continuation-in-part of application No. PCT/US2006/039267, filed on Oct. 6, 2006.
Application No. PCT/US2006/039267 is a continuation-in-part of application No. 11/539,613, filed on Oct. 6, 2006.
(60) Provisional application No. 60/617,500, filed on Oct. 8, 2004.

(51) Int. Cl. A61K 38/16; C07K 14/00; C12P 21/00; C12N 15/74; C12N 5/10; A61P 3/10

(52) U.S. Cl. 514/12; 530/324; 530/335; 435/320.1; 435/325

(57) ABSTRACT

Pharmaceutical compositions relating to vasoactive intestinal polypeptides and methods for the treatment of metabolic disorders, including diabetes, insulin resistance, metabolic acidosis and obesity are presented. Methods of using the vasoactive intestinal polypeptide compositions are also disclosed.

### CROSS REFERENCE TO RELATED APPLICATIONS

This application is a continuation of U.S. application Ser. No. 12/093,195, filed May 9, 2008, which was a 371 of International Application No. PCT/US2006/039267, filed Oct. 6, 2006, which is a continuation-in-part of U.S. application Ser. No. 11/279,238, filed Apr. 10, 2006, and U.S. application Ser. No. 11/245,499 filed Oct. 7, 2005. This application is a continuation-in-part of U.S. application Ser. No. 11/869,032, filed Oct. 9, 2007, which is a continuation-in-part of U.S. application Ser. No. 11/539,613, filed Oct. 6, 2006, which is a continuation-in-part of U.S. application Ser. No. 11/279,238, filed Apr. 10, 2006, which is a continuation-in-part of U.S. application Ser. No. 11/245,499 filed Oct. 7, 2005, which claims benefit of U.S. Provisional Application No. 60/617,500 filed Oct. 8, 2004, now abandoned. The contents of these above-mentioned applications are incorporated by reference herein in their entireties.

### FIELD OF THE INVENTION

The invention relates to polypeptide analogs and their synthesis and uses. More particularly, the invention relates to synthetic polypeptide analogs related to vasoactive intestinal polypeptide, and pharmaceutical compositions thereof.

### BACKGROUND

When food is present in the alimentary canal, cells in the gut secrete a hormonal signal (an “incretin”), which sensitizes the pancreas to the presence of glucose and results in a potentiated glucose-dependent insulin secretory response. Such a synergistic response to provide glucose-dependent insulin release (Kieffer T J and Habener, J R., Endocr. Rev. 20, 876-913 (1999)) is seen for the incretin signals, Glucagon-like Peptide 1 (GLP1) and Glucose-dependent Insulinotropic Peptide (GIP). These incretin signals typically exhibit short duration of action in the body, with GLP1 exhibiting a t_{1/2} of approximately 1-2 minutes (Knudsen, L B., J. Med. Chem. 47, 4128-34 (2004)). GLP1 and GIP are cleaved by an amino peptidase, dipeptidyl peptidase IV (DPPIV) and thus, the naturally occurring native hormone is not generally used in medicinal formulations. A peptide found in the saliva of the Gila Monster (exendin 4, Exenatide, BYETTA®; Amylin Pharmaceuticals Inc., San Diego, Calif.) was shown to bind to the GLP1 receptor and exhibit potent agonistic activity (Young, A A, et al., Diabetes, 48: 1026-34 (1999)), thereby imparting a desirable glucose-dependent insulin secretory response (Nielsen L L, Young, A A, Parkes, D G., Regul. Peptides, 117, 77-88 (2004)). Exenatide and analogs of GLP1 have been administered to patients in need of treatment for type 2 diabetes.

Pituitary Adenylate Cyclase-Activating Peptide (PACAP) is a neuromodulatory peptide which stimulates PAC1, VPAC1, and VPAC2 receptors, and is emitted from nerve endings in the pancreas. Receptors of this general class reside in multiple tissues in the body, including in the pancreas (Vaudry D, et al. Pharmacol Rev 52: 269-324 (2000)). PACAP is believed to participate in the physiological response to food in the gut and thus appears to be complementary to the hormonal, incretin response (Filipsson, K., et al., Am. J. Physiol. Regulatory Integrative Comp. Physiol. 279: R424-32 (2000)). Administration (infusion) of PACAP to human volunteers or to rodents causes potentiated glucose-dependent insulin secretion, but also results in hyperglycemia (Filipsson K, Tornoe K, Holst J and Ahren B., J Clin Endocrinol Metab 82: 3093-8 (1997)). In contrast, Vasoactive Intestinal Polypeptide (VIP) activates only the VPAC1 and VPAC2 receptors. In the pancreas, stimulation of the VPAC2 receptors has been shown to provide a potentiated, glucose-dependent insulin release in response to elevated blood glucose levels similar to that of GLP1 or exenatide (Tsutsumi, M., et al., Diabetes 51, 1453-60 (2002)). Furthermore, VPAC2 receptors are present on human pancreatic beta cells. Thus, in view of the complementary physiological role of PACAP, such a stimulus (from PACAP or VPAC agonistic analogs) could be synergistic or alternative to incretin-like signals in stimulating glucose-dependent insulin release, since a similar profile of potentiated insulin secretion results from activation of a second class of receptor. Such an effect would be beneficial in the treatment of metabolic disorders, including Type 2 Diabetes Mellitus (T2DM), metabolic acidosis, insulin resistance and obesity. However, the lack of blood glucose lowering by PACAP in vivo is thought to be related to its ability to cause gluconeogenesis in the liver and release of glucagon. These activities, as well as several side effects (watery diarrhea, hypotension, hepatic gluconeogenesis), are believed to be caused by activation of PAC1 and VPAC1 receptors (Tsutsumi, M., et al., Diabetes 51, 1453-60 (2002)). It was therefore determined that a VPAC2 modulatory ligand could have beneficial effects in the treatment of T2DM and have a reduced side effect profile. In addition, the naturally occurring native sequence of PACAP and its analogs also are typically short-lived in the body. Therefore there is an important medical need for selective VPAC2 modulators. VPAC2 modulators can be either VPAC2 agonists or antagonists.

Another reptile hormone-like molecule, Heliodermin (SEQ ID NO: 80), exhibits great selectivity for the VPAC2 rather than for the VPAC1 receptor (Gourlet, P., et al. Ann. NY Acad. Sci. 865: 247-52 (1998)). Certain substitutions, such as Gln at positions 8 and 9, as well as Leu-Ala-Lys at positions 14 through 16 may have particular significance for receptor selectivity. However the reptile peptides, being foreign to the human body, can be highly antigenic in man. Although the reptile GLP1 like molecule is longer acting than the mammalian incretins, synthetic exendin-4 (BYETTA® Amylin Pharmaceuticals, Inc., San Diego, Calif.) remains a relatively short acting peptide (t_{1/2} 2 hr in man) and there is a medical need for longer-acting peptides that can modulate glucose-dependent insulin secretion.

Treatment of preconstricted smooth muscle preparations from the lungs of animals and humans with VPAC2 agonists results in prompt relaxation (O'Donnell, K., et al., J. Pharmacol. Exptl. Therapeut. 270: 1282-8 (1994)). Similarly, treatment of asthma patients with a VPAC2 agonist has been reported to result in prompt bronchodilatation (Linden, A., et al. Thorax 58: 217-21 (2003)).

### SUMMARY OF THE INVENTION

In one aspect, synthetic polypeptide analogs of PACAP and Vasoactive Intestinal Polypeptide (VIP), and salts thereof are provided, in which the C-terminus comprises amino acid residues that form an amphipathic α-helix, said residues selected from hydrophilic amino acids (Haa) and lipophilic amino acids (Laa) ordered in the sequence:

(SEQ ID NOS: 83-87)

(Laa Laa Haa Haa)ₙ, Laa, wherein n = 1-5
(hereinafter Formula A).
In an embodiment, n = 1 or 2.

In another embodiment, said residues selected from hydrophilic amino acids (Haa) and lipophilic amino acids (Laa) are ordered in the sequence:

(SEQ ID NOS: 88, 409-412)

Haa (Laa Laa Haa Haa)ₙ, Laa, wherein n = 1-5
(hereinafter Formula B).
In an embodiment, n = 1 or 2.

Modifications introduced in the present polypeptide analogs of PACAP and VIP facilitate increased duration of action of therapeutics which activate the PACAP and VIP family of receptors, preferably the VPAC2 receptor. Without being bound to any particular theory, it is believed that an increase in duration of action may be due to the ability of the amphipathic helix in the C-terminal region to interact with the phospholipids of the cell membranes in the body and thereby have a “depoting” effect. Thus, the present peptide analogs are thought to bind to cell membranes and then slowly re-release to the plasma to impart its effect distally. In contrast, if a peptide such as PACAP, VIP or GLP1 is free in the plasma it is rapidly acted upon by proteases or cleared by glomerular filtration into the urine (Nestor J J Jr., Improved Duration of Action of Peptide Drugs. In Peptide-based Drug Design: Taylor M D, Amidon G L, Eds.; American Chemical Society Washington D.C., 1995: 449-471).

Therefore, in one aspect analogs to PACAP and/or VIP, and the physiologically active truncated analogs and homologs of same, or salts thereof are provided, in which the C-terminus preferably comprises amino acid residues that form an amphipathic α-helix, the sequence of said residues selected from the native amino acids or selected unnatural amino acids having the ability to stabilize said α-helix.

Also provided are pharmaceutical compositions for the delivery of an effective glucose-dependant insulin releasing amount of a polypeptide analog of PACAP and/or VIP, and the physiologically active truncated analogs and homologs of same, or a salt thereof, in which the C-terminus preferably comprises amino acid residues that form an amphipathic α-helix, said residues selected from hydrophilic amino acids (Haa) and lipophilic amino acids (Laa) ordered in the sequence of Formula A.

In another embodiment, said residues selected from hydrophilic amino acids (Haa) and lipophilic amino acids (Laa) are ordered in the sequence of Formula B.

In another aspect, methods for treating mammalian conditions characterized by high blood glucose are provided, which methods comprise administering to a mammal in need thereof an effective glucose-dependant insulin releasing amount of a polypeptide analog of PACAP and/or VIP, and the physiologically active truncated analogs and homologs of same, or a salt thereof, in which the C-terminus preferably comprises amino acid that form an amphipathic α-helix, said residues selected from hydrophilic amino acids (Haa) and lipophilic amino acids (Laa) ordered in the sequence of Formula A. In an embodiment, n=1 or 2.

In another embodiment, said residues selected from hydrophilic amino acids (Haa) and lipophilic amino acids (Laa) are ordered in the sequence of Formula B. In an embodiment, n=1 or 2.

In another aspect, methods for treating mammalian conditions affected by VPAC receptor activation are provided, which methods comprise administering to a mammal in need thereof an effective glucose-dependant insulin releasing amount of a polypeptide analog of PACAP and/or VIP, and the physiologically active truncated analogs and homologs of same, or a salt thereof, in which the C-terminus preferably comprises amino acid that form an amphipathic α-helix, said residues selected from hydrophilic amino acids (Haa) and lipophilic amino acids (Laa) ordered in the sequence of Formula A. In an embodiment, n=1 or 2.

In another embodiment, said residues selected from hydrophilic amino acids (Haa) and lipophilic amino acids (Laa) are ordered in the sequence of Formula B. In an embodiment, n=1 or 2.

Processes are provided for the solid phase synthesis of polypeptide analogs of PACAP and/or VIP, and the physiologically active truncated analogs and homologs of same, or a salt thereof, in which the C-terminus preferably comprises amino acid residues that form an amphipathic α-helix, said residues selected from hydrophilic amino acids (Haa), and lipophilic amino acids (Laa) ordered in the sequence of Formula A. In an embodiment, n=1 or 2.

In another embodiment, said residues selected from hydrophilic amino acids (Haa) and lipophilic amino acids (Laa) are ordered in the sequence of Formula B. In an embodiment, n=1 or 2.

Processes presented herein for preparing polypeptide analogs comprise sequentially coupling protected amino acids on a suitable resin support, removing the side chain and Nα-protecting groups, and cleaving the polypeptide from the resin.

In further or alternative embodiments, the method further comprising the step of using microwave assistance. In further or alternative embodiments, the microwave assistance is used for synthesizing polypeptides containing at least one amino acid which is not one of the twenty standard amino acids.

Another embodiment provides DNA sequences, vectors, and plasmids for the recombinant synthesis of polypeptide analogs of PACAP and/or VIP, and the physiologically active truncated analogs and homologs of same, or a salt thereof, in which the C-terminus comprises amino acid residues that form an amphipathic α-helix, said residues selected from hydrophilic amino acids (Haa) and lipophilic amino acids (Laa) ordered in the sequence of Formula A. In an embodiment, n=1 or 2.

In another embodiment, said residues selected from hydrophilic amino acids (Haa) and lipophilic amino acids (Laa) are ordered in the sequence of Formula B. In an embodiment, n=1 or 2.

Another aspect provides pharmaceutical compositions and methods for the prevention and treatment of a variety of diseases and disorders including, but not limited to: metabolic disorders, including diabetes, insulin resistance, hyperglycemia, metabolic acidosis and obesity, which are manifested by elevated blood glucose levels, dyslipidemia, hypertriglyceridemia and obesity, as well as chronic obstructive pulmonary disease, cardioprotection during ischemia, primary pulmonary hypertension and asthma, comprising an effective amount of these described polypeptide(s), or salt thereof, and a pharmaceutically acceptable carrier. In other aspects, therapeutically effective amounts of metabolic disorder compounds, including insulin, insulin analogs, incretin, incretin analogs, glucagon-like peptide, glucagon-like peptide analogs, glucose dependent insulinotropic peptide analogs, exendin, exendin analogs, sulfonylureas, meglitinides, biguanides, α-glucosidase inhibitors, thiazolidinediones, peroxisome proliferator activated receptor (PPAR) agonists, PPAR antagonists and PPAR partial agonists may be administered in combination with the described polypeptides. In yet other aspects, therapeutically effective amounts of various other agents useful for the prevention and treatment of the aforementioned diseases and disorders, and described further hereinbelow, may be administered in combination with the described polypeptides.

### BRIEF DESCRIPTION OF THE DRAWINGS

FIGS. 1A, 1B, 1C, 1D, and 1E are lists of exemplary polypeptide analogs described herein. In this and other figures and throughout this specification, unless otherwise provided, standard nomenclature using single letter abbreviations for amino acids are used. In certain embodiments, the letter “X” refers to a polyethylene glycol chain or PEG having C₁₀-C₃₀₀₀ chain. Preferred polyethylene glycol chains may be linear or branched and will have a molecular weight above 20 kiloDalton. In another embodiment, the polyethylene glycol chain will have a molecular weight of 250 to 5,000 Da, preferably from 500 to 2,000 Da. The term “acyl” refers to a C₂-C₃₀ acyl chain. This chain may comprise a linear aliphatic chain, a branched aliphatic chain, an aralkyl chain, or an aryl chain containing an acyl moiety. The letter “Z” refers to lysine having a long acyl chain at the epsilon position. For clarity, when the Z is at the C-terminus, it also denotes the presence of an amide C-terminus as described below as X (that is NHR1), unless otherwise noted. When it is not at the C-terminus it denotes an episilon-modified lysine residue. The term “hex” refers to hexanoyl. The term “open” refers to pentanoyl. The terms “lau” refers to lauroyl. The term “myr” refers to myristoyl. The term “step” refers to stearoyl. The term “pr” refers to propionyl. Arachidoyl refers to a linear C20 saturated fatty acid substituent (i.e. 20:0). The term “Be” refers to behenoyl (22:0), “Er” to erucoyl (22:1), and “Ner” to nervonyl (24:1).

FIG. 2 lists other polypeptide and polypeptide analogs.

FIGS. 3A, 3B, 3C, 3D, 3E, 3F, 3G, 3H, 3J, 3K, 3L, 3M, 3N, 3P, 3Q and 3R list additional exemplary polypeptide analogs described herein.

FIGS. 4A and 4B list preferred compounds described herein. FIGS. 4C, 4D, 4E, 4F, 4G, and 4H list additional exemplary polypeptide analogs described herein.

FIGS. 5A to 5M list additional exemplary polypeptide analogs.

### DETAILED DESCRIPTION OF THE INVENTION

### Abbreviations and Definitions

The one- and three-letter abbreviations for the various common nucleotide bases and amino acids are as recommended in Pure Appl. Chem. 31, 639-645 (1972) and 40, 277-290 (1974) and comply with 37 CFR § 1.822 (55 FR 18245, May 1, 1990). The abbreviations represent L-amino acids unless otherwise designated as D- or DL. Certain amino acids, both natural and non-natural, are achiral, e.g., glycine. All peptide sequences are presented with the N-terminal amino acid on the left and the C-terminal amino acid on the right.

“Hydrophilic amino acid (Haa)” refers to an amino acid having at least one hydrophilic functional group in addition to those required for peptide bond formation, such as, but not limited to, arginine, asparagine, aspartic acid, glutamic acid, glutamine, histidine, lysine, serine, threonine, and their homologs.

“Lipophilic amino acid (Laa)” refers to an uncharged, aliphatic or aromatic amino acid, such as, but not limited to, isoleucine, leucine, methionine, phenylalanine, tryptophan, tyrosine, valine, and their homologs.

In this specification, alanine is classified as “ambiphilic” i.e., capable of acting as either hydrophilic or lipophilic.

“Homolog of PACAP or VIP” refers to a polypeptide comprising amino acids in a sequence that is substantially similar to the native sequence of PACAP or VIP, such as at least 50, 60, 70, 80, 85, 90, 91, 92, 93, 94, 95, 96, 97, 98, or 99% amino acid sequence identity. Homologs presented herein may comprise amino acid substitutions, deletions, and/or insertions relative to the native sequence of PACAP or VIP. Exemplary homologs comprise a span of at least 5, 10, 15, 20, 25, 30, or 35 contiguous amino acids that are identical or substantially similar to the native sequence of PACAP or VIP.

“Analogs of PACAP or VIP” refers to a polypeptide comprising: (i) PACAP, VIP, and/or homologs of PACAP or VIP; and (ii) at least one functionality not present in naturally occurring native PACAP and/or VIP. For example, analogs can optionally comprise a functionality within the sidechain of an amino acid or at the amino or carboxyl terminal of the polypeptide. Exemplary functionalities include alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amino group, or the like or any combination thereof. Other exemplary functionalities that can be introduced include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analogue, glycosylated amino acids such as a sugar substituted serine, other carbohydrate modified amino acids, keto containing amino acids, amino acids comprising polyethylene glycol or polyether, heavy atom substituted amino acids, chemically cleavable and/or photocleavable amino acids, amino acids with an elongated side chains as compared to natural amino acids, e.g., polyethers or long chain hydrocarbons, e.g., greater than about 5 or greater than about 10 carbons, carbon-linked sugar-containing amino acids, redox-active amino acids, amino thioacid containing amino acids, and amino acids comprising one or more toxic moiety.

Analogs presented herein may comprise non-natural amino acids based on natural amino acids, such as tyrosine analogs include para-substituted tyrosines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, or the like. Glutamine analogs include, but are not limited to, α-hydroxy derivatives, β-substituted derivatives, cyclic derivatives, and amide substituted glutamine derivatives. Examples of phenylalanine analogs include, but are not limited to, meta-substituted phenylalanines, wherein the substituent comprises a hydroxy group, a methoxy group, a methyl group, an allyl group, an acetyl group, or the like. Specific examples include, but are not limited to, α-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAc-β-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

Generally, analogs are optionally designed or selected to modify the biological properties of the polypeptide, such as to modulate toxicity, biodistribution, solubility, stability, e.g., thermal, hydrolytic, oxidative, resistance to enzymatic degradation, and the like, facility of purification and processing, structural properties, spectroscopic properties, chemical and/or photochemical properties, catalytic activity, redox potential, half-life, ability to react with other molecules, e.g., covalently or noncovalently, and the like.

One type of modification is designed to block proteolysis in the tissues. For example, it is known that the proteolytic pattern for VIP administered to inflamed lungs shows rapid cleavage by a trypsin-like enzyme at the Arg residue at position Arg¹⁴ to give largely VIP1-14 (Lilly, C. M., et al., J. Clin. Invest. 93: 2667-74 (1994)). Thus substitution by a non-basic amino acid at this position would block this principal clearance route. The use of portions of the sequence found in Heliodermin in this region (Leu¹³-Leu-Ala-Lys-Leu-Ala-Leu-Gln²⁰ (SEQ ID NO: 82)) is therefore a desirable modification, especially for development of treatments for inflammatory lung diseases like asthma and COPD. Particularly preferred is the use of Leu at position 14.

“Physiologically active truncated homolog or analog of PACAP or VIP” refers to a polypeptide having a sequence comprising less than the full complement of amino acids found in PACAP or VIP which, however, elicits a similar physiological response. Representative truncated homologs and/or analogs presented herein comprise at least 5, 10, 15, 20, 25, 30, or 35 contiguous amino acids found in the native sequence of PACAP or VIP. The truncated PACAP or VIP need not be fully homologous with PACAP or VIP to elicit a similar physiological response. PACAP or VIP are preferred, but not exclusive, representatives of this group.

“PEG” refers to polyethylene glycol, polypropylene glycol, or polyoxyalkylenes attached to the peptide or protein through a linker functional group (see reviews—Veronese, F. M., et al., Drug Disc. Today 10: 1451-8 (2005); Greenwald, R. B., et al., Adv. Drug Deliv. Rev. 55: 217-50 (2003); Roberts, M. J., et al., Adv. Drug Deliv. Rev., 54: 459-76 (2002)). PEG-modified (PEGylated) proteins or peptides can exhibit very beneficial characteristics such as very prolonged duration of action and reduced antigenicity, following parenteral delivery. These beneficial characteristics are believed to be due in part to a decreased recognition by proteases and the reticuloendothelial system due to a shielding effect by the PEG chain. Another very important mechanism is by increasing the apparent molecular weight so that it becomes greater than the cutoff for filtration through the glomerular barrier in the kidney and into the urine. This cutoff size is near that of serum albumin (about 60 kDa). The highly hydrated character of the PEG chain causes it to have an “effective molecular weight” with respect to glomerular filtration like that of a globular protein more than three times larger than its true molecular weight. Thus for prolongation of duration of action following parenteral administration, preferred forms of PEG for use herein have a molecular weight of greater than 10,000 Da and most preferred forms have a molecular weight of 20,000 Da or greater. PEG chains may be linear or branched molecules.

Another type of PEG chain is modified to be amphiphilic in nature. That is it has both the hydrophilic PEG structure but is modified to contain hydrophobic regions such as fatty acid esters and other hydrophobic components (see for example Miller, M. A., et al., Bioconjug. Chem. 17: 267-74 (2006); Ekwuribe, et al. U.S. Pat. No. 6,309,633; Ekwuribe, et al. U.S. Pat. No. 6,815,530; Ekwuribe, et al. U.S. Pat. No. 6,835,802). Although these amphiphilic PEG conjugates to proteins were originally developed to increase oral bioavailability they were relatively ineffective in this role. However the use of such amphiphilic PEG conjugates with the amphipathic peptides described herein will give significantly prolonged residence in the lung to extend the useful biological activity of these pharmaceuticals. The preferred PEG chains are in the molecular weight range of 500 to 3000 Da. Detailed descriptions of the methods of synthesis of these conjugates is given in the references above, the full content of which is incorporated herein.

Another type of PEG modification uses monodisperse or discrete PEGs. Thus while earlier, conventional PEG molecules were made by polymerization to yield mixtures of molecules within a relatively broad range of molecular weights, Quanta BioDesign (Powell, Ohio) has generated reagents with a single molecular weight, designated discrete PEGs. These latter reagents are felt to have certain advantages due the homogeneity of the product formed. Such discrete PEGylated products will be easier to characterize and may be more reproducible to produce. In the examples the reagents of this class are denoted m-d PEG for the methoxy-discrete PEG class. Various types of linkage to the peptide chain are possible with these and other PEG units. Preferred linkages are through a cysteine residue using a maleimide moiety on the PEG or through a lysine ε-amino function using an acid linkage on the PEG.

PEGylation of a protein (that is, reaction with various functionalized PEG chains to incorporate PEG into the structure) can have potentially negative effects as well. Thus PEGylation can cause a substantial loss of biological activity for some proteins and this may relate to ligands for specific classes of receptors. In such instances there is a benefit to reversible PEGylation (Peleg-Shulman, T., et al., J. Med. Chem. 47: 4897-4904; Greenwald, R. B., et al. Adv. Drug Del. Rev., 55: 217-50)).

In addition, the increased molecular mass may prevent penetration of physiological barriers other than the glomerular membrane barrier. For example, it has been suggested that high molecular weight forms of PEGylation may prevent penetration to some tissues and thereby reduce therapeutic efficacy. In addition, high molecular weight may prevent uptake across mucosal membrane barriers (nasal, buccal, vaginal, oral, rectal, lung delivery). However delayed uptake may be highly advantageous for administration of stable molecules to the lung, substantially prolonging the duration of action.

An important aspect is the use of not just long chain PEG polymers, but the use of short chain versions as well. Administration of treatments for diabetes by inhalation is an important new approach for drug delivery and the lung has a highly permeable barrier (e.g. Exubera). For this application, delayed penetration of the lung barrier, preferred forms of PEGylation are in the lower molecular weight range of C₁₀ to C₄₀₀ (roughly 250 to 10,000 Da). Thus while a primary route to prolongation by PEG is the achievement of an “effective molecular weight” above the glomerular filtration cut-off (greater than 60 kDa) and this is the preponderant use of PEG, use of shorter chains, as illustrated here, may be an important route for prolongation of residence in the lung for treatment of lung diseases and other respiratory conditions. Thus PEG chains of about 500 to 3000 Da are of sufficient size to slow the entry into the peripheral circulation, but insufficient to cause them to have a very prolonged circulation time, and are preferred in certain embodiments. Shorter PEG chains have clear advantages for application to the lung, while longer PEG chains may not be cleared well from the lung or the systemic circulation. Thus, in these embodiments, PEGylation may be applied to give increased local efficacy to the lung tissue with reduced potential for systemic side effects for the compounds described herein. As used herein, those PEG chains in the range from about 750 to about 1500 Da are referred collectively as “PG1K.” While PEG of 2000 Da average molecular weight also fall within the “PG1k” definition, in specific instances herein, they may be denoted PG2k.

Polyethylene glycol chains are functionalized to allow their conjugation to reactive groups on the polypeptide or protein chain. Typical functional groups allow reaction with amino, carboxy or sulfhydryl groups on the peptide through the corresponding carboxy, amino or maleimido groups (and the like) on the polyethylene glycol chain. In an embodiment, PEG comprises a C₁₀-C₃₀₀₀ chain. In another embodiment, PEG has a molecular weight above 40,000 Daltons. In yet another embodiment, PEG has a molecular weight below 10,000 Daltons. PEG as a protein modification is well known in the art and its use is described, for example, in U.S. Pat. Nos. 4,640,835; 4,496,689; 4,301,144; 4,670,417; 4,791,192; and 4,179,337.

“Amphipathic α-helix” refers to the secondary structure exhibited by certain polypeptides in which the amino acids assume a α-helical configuration having opposing polar and nonpolar faces oriented along the long axis of the helix. Various authors use the terms amphipathic or amphiphilic α-helix interchangeably in that one face is polar and one is nonpolar, and both terms are used to mean the same type of structure herein.

The possibility of α-helical structure in the polypeptide of interest may be explored to some extent by the construction of a “Schiffer-Edmundson wheel” (Schiffer, M. and Edmundson, A. B., Biophys. J. 7, 121 (1967), incorporated herein by reference), of the appropriate pitch and noting the segregation of the hydrophilic and lipophilic residues on opposite faces of the cylinder circumscribing the helix. Alternatively, empirical evidence, such as circular dichroism or x-ray diffraction data, may be available indicating the presence of an α-helical region in a given polypeptide. An ideal α-helix has 3.6 amino acid residues per turn with adjacent side chains separated by 100° of arc.

Another aspect of protein structure relevant to certain polypeptides described herein, and in particular those compounds of the structure corresponding to Formula C (SEQ ID NO: 81) or modifications thereof, is the use of a polyproline type II helix (Stapley, B. J. and Creamer, T. P., Protein Sci 8: 587-95 (1999)) to facilitate the development of the amphipathic a helix described above. Polyproline helices increasingly are recognized as being an important element in protein structure and an important aspect of that helix is its amphiphilic character. Here we make use of such a polyproline type II helix to facilitate that formation of the amphipathic a helix described above to yield potent VPAC2 ligands. A prominent feature of polyproline helices is the very strong preference for Pro residues within the helix and specific amino acids as capping residues at the N-terminus. Some examples of favored capping residues are Gln, Ser, Gly, Asp, Ala, Arg, Lys, Glu (Rucker A L, et al., Proteins 53: 68-75 (2003)).

Another aspect of the polyproline helix is the resistance to proteolysis that it affords. A number of naturally occurring peptides and proteins have polyproline regions or Pro residues at their C-terminus, where they may also prevent proteolytic digestion. Examples that bind to the GLP1 receptor are Exendin-4, heliodermin, and heliospectin.

Unless stated otherwise, standard nomenclature using single letter abbreviations for amino acids are used. The letter “X” refers to a polyethylene glycol chain having C₁₀-C₃₀₀₀ chain. Preferred polyethylene glycol chains may be linear or branched and will have a molecular weight above 20 kiloDalton. In another embodiment, the polyethylene chain will have a molecular weight of from about 250 to about 5,000 Da, preferably from about 500 to about 2,000 Da. The term “acyl” refers to a C₂-C₃₀ acyl chain. This chain may comprise a linear aliphatic chain, a branched aliphatic chain, an aralkyl chain, or an aryl chain containing an acyl moiety. The letter “Z” refers to lysine having a long acyl chain at the epsilon position. For clarity, when the Z is at the C-terminus, it also denotes the presence of an amide C-terminus as described below as X (that is NHR1), unless otherwise noted. When it is not at the C-terminus it denotes an episilon-modified lysine residue. The term “hex” refers to hexanoyl. The term “pen” refers to pentanoyl. The terms “lau” refers to lauroyl. The term “myr” refers to myristoyl. The term “step” refers to stearoyl. The term “pr” refers to propionyl. Arachidoyl refers to a linear C20 saturated fatty acid substituent (i.e. 20:0). The term “Be” refers to behenoyl (22:0), “Er” to erucoyl (22:1), and “Ner” to nervonyl (24:1). For example, in SEQ ID NO: 25, the “Z myr” represents “Lys(epsilon myristoyl),” making the sequence end Leu-Lys(epsilon myristoyl)-Pro-Pro-Pro.

Although it may be apparent to an ordinary person skilled in the art, a PEG entity itself does not have a functional group to be attached to a target molecule, such as a peptide. Therefore, to create PEG attachment, a PEG entity must be functionalized first, then a functionalized attachment is used to attach the PEG entity to a target molecule, such as a peptide (Veronese, F. M., et al., Drug Disc. Today 10: 1451-8 (2005); Greenwald, R. B., et al., Adv. Drug Deliv. Rev. 55: 217-50 (2003); Roberts, M. J., et al., Adv. Drug Deliv. Rev., 54: 459-76 (2002)). In one embodiment, site-specific PEGylation can be achieved through Cys substitution on a peptide molecule. The target peptide can be synthesized by solid phase synthesis, recombinant means, and other means, as described herein. One embodiment of the invention discloses the combination concept of using acylation on a Lys residue and specific PEGylation on at least one Cys residue. Certain Lys residues in disclosed peptide sequences can be substituted to Cys for site-specific PEGylation.

In another embodiment, a Lys or other residue residue with a nucleophilic side chain may be used for incorporation of a PEG residue. This may be accomplished through the use of an amide or carbamate linkage to a PEG-carboxyl or PEG-carbonate chain (for example, as described in Veronese, F. M., et al. Drug Dise. Today 10: 1451-8 (2005)). An alternative approach is to modify the Lys side chain amino function through attachment of an SH containing residue, such as mercaptoacetyl, mercaptopropionyl (CO—CH₂—CH₂—CH₂—SH), and the like. Additional methods for attaching PEG chains utilize reaction with the side chains of His and Trp. Other similar methods of modifying the peptide chain to allow attachment of a PEG chain are known in the art and are incorporated herein by reference.

Aside from the twenty standard amino acids, there are a vast number of “nonstandard amino acids” which exist in various life forms that may be incorporated in the compounds described herein. Examples of nonstandard amino acids include the sulfur-containing taurine and the neurotransmitters GABA and dopamine. Other examples are lanthionine, 2-Aminoisobutyric acid (Aib), and dehydroalanine. Nonstandard amino acids often occur in the metabolic pathways for standard amino acids—for example ornithine (Orn) and citrulline (Cit) occur in the urea cycle, part of amino acid breakdown.

The term “naturally occurring amino acid” as used herein includes both twenty standard amino acids and other nonstandard amino acid, including, but not limited to, Aib, Orn, and Cit.

### Polypeptides

In an embodiment, polypeptides presented herein comprise truncated portions of PACAP and/or VIP having at least 5, 10, 15, 20, 25, 30, or 35 contiguous amino acids of the native sequence of PACAP or VIP. In another embodiment, the present polypeptides share at least 50, 60, 70, 80, 85, 90, 95, or 99% amino acid sequence identity to the native sequence of PACAP or VIP. In yet another embodiment, the present polypeptides comprise a span of at least 5, 10, 15, 20, 25, 30, or contiguous amino acids of PACAP and/or VIP having at least 50, 60, 70, 80, 85, 90, 91, 92, 93, 94, 95, 96, 97, 98, 99, or 100% amino acid sequence identity to the native sequence of PACAP or VIP.

One type of modification is designed to block proteolysis in the tissues. For example, it is known that the proteolytic pattern for VIP administered to inflamed lungs shows rapid cleavage by a trypsin-like enzyme at the Arg residue at position Arg¹⁴ to give largely VIP1-14 (Lilly, C. M., et al., J. Clin. Invest. 93: 2667-74 (1994)). Thus substitution by a non-basic amino acid at this position would block this principal clearance route. The use of portions of the sequence found in Heliodermin in this region (Leu¹³-Leu-Ala-Lys-Leu-Ala-Leu-Gln²⁰) (SEQ ID NO: 82) is therefore a desirable modification, especially for development of treatments for inflammatory lung diseases like asthma and COPD. Particularly preferred is the use of Leu at position 14. Certain substitutions, such as Gln at positions 8 and 9, as well as Leu-Ala-Lys at positions 14 through 16 may have particular significance for receptor selectivity.

Polypeptides presented herein optionally comprise modifications, functionalities, and/or amino acid substitutions which modulate VPAC2 selectivity. Exemplary modifications, functionalities, and/or substitutions include, but are not limited to, C-terminal cationic extensions and/or mutations (Gourlet et al., Peptides 18, 403-8; (1997)); Xia M, et al., J. Pharmacol. Exp. Ther. 281: 629-33 (1997); the contents of both of which are incorporated herein by reference).

Modifications at the amino or carboxyl terminus may optionally be introduced into the present polypeptides. For example, the present polypeptides, such as analogs of VIP, can be acylated on the N-terminus by long chain fatty acids to yield polypeptides exhibiting low efficacy, partial agonist and antagonist activity (Gourlet et al., Eur. J. Pharmacol. 354: 105-111 (1998)), the contents of which are incorporated herein by reference). Modification of the peptides described herein with longer chain fatty acids at the N-terminus, similarly will yield antagonists with a prolonged duration of action (Moreno D, et al., Peptides 21: 1543-9 (2000)). Other modifications to the N-terminus, such as deletions or incorporation of D-amino acids such as D-Phe also give potent and long acting antagonists when substituted into the compounds of Formulae C and D. Such antagonists also have commercial utility and are within the scope of this invention.

Other exemplary modifications of the present polypeptides, such as analogs of VIP and/or PACAP, include acylation with hexanoic acid to yield polypeptides that exhibit increased selectivity towards VPAC2 (Langer et al., Peptides 25: 275-8 (2004)), the contents of which is incorporated herein by reference). Thus the length and positioning of such acylation is important since it can alter efficacy, and could result in loss of efficacy (antagonistic) or agonistic analogs. Contrary to this unpredictability, polypeptides of the type presented herein have been designed and tested to obtain desired efficacy and activity.

Another very favorable aspect of N-terminal acylation is the blockade of rapid proteolysis by DPPIV seen for the parent peptide due to such acylation. Thus although PACAP and VIP have very short duration of action in vivo, the peptides described herein preferably have a principal proteolysis route blocked by this N-terminal acylation.

Modifications may optionally be introduced within the side chain of at least one amino acid within the present polypeptides to increase duration of action and/or potency. For example, the present polypeptides can optionally comprise at least one amino acid acylated to a functionality in the side chain (i.e., R group). Representative modifications include fatty acid acylation, directly or through linkers, of reactive side chains (such as Lys) at various positions within the polypeptide. Similar modifications have been reported in Kurtzhals et al. where acylation of insulin on LysB²⁹ resulted in insulin detemir (Kurtzhals et al., Biochem J. 312, 725-31 (1995) and Kurtzhals, P., Int. J. Obesity 28: Suppl 2, S23-8 (2004)). Similarly, acylation with long chain fatty acids through linkers (preferably Glu) has resulted in potent and long-acting analogs of GLP1 (Knudsen L. B., et al., J. Med. Chem. 43:1664-69 (2000)), but the acylation can result in loss of activity or potent agonists, depending on the length and positioning of the acyl chain(s). Contrary to the unpredictable effects with the introduction of long chain fatty acids, polypeptides presented herein have been designed to incorporate an optimal number, length and positioning of the acyl chains so as to obtain desired activity. Such linkage is demonstrated here for direct acylation to Lys, but linkage through other linkers, such as Glu (Knudsen, L B, et al., J. Med. Chem. 43: 1664-9 (2000)), is also within the scope of the present invention.

Another type of modification that can optionally be introduced into the present polypeptides (e.g. within the polypeptide chain or at either the N- or C-terminal) to extend duration of action is PEGylation or incorporation of long-chain polyethylene glycol polymers (PEG). Introduction of PEG or long chain polymers of PEG increases the effective molecular weight of the present polypeptides to prevent rapid filtration into the urine. Any Lys residue in any peptide analog sequence may be conjugated to PEG directly or through a linker to yield a potent and long acting analog. Such linker can be a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are well known in the art (described, for example, in Veronese, F. M., et al., Drug Disc. Today 10: 1451-8 (2005); Greenwald, R. B., et al., Adv. Drug Deliv. Rev. 55: 217-50 (2003); Roberts, M. J., et al., Adv. Drug Deliv. Rev., 54: 459-76 (2002)), the contents of which is incorporated herein by reference.

A more recently reported alternative approach for incorporating PEG or PEG polymers through incorporation of non-natural amino acids can be performed with the present polypeptides. This approach utilizes an evolved tRNA/tRNA synthetase pair and is coded in the expression plasmid by the amber suppressor codon (Deiters, A, et al. (2004). Bio-org. Med. Chem. Lett. 14, 5743-5). For example, p-azidophenylalanine can be incorporated into the present polypeptides and then reacted with a PEG polymer having an acetylene moiety in the presence of a reducing agent and copper ions to facilitate an organic reaction known as “Huisgen [3+2]cycloaddition.”

### Amphipathic Helix

In one aspect or embodiment, certain described polypeptides comprise an amphipathic α-helix corresponding to the formula:

(SEQ ID NOS: 83-87)

(Laa Laa Haa Haa)ₙ, Laa, wherein n = 1-5
(Formula A).

Each Haa is independently selected from the group of hydrophilic amino acids and each Laa is independently selected from the group of lipophilic amino acids, as defined above.

In another embodiment, said residues selected from hydrophilic amino acids (Haa) and lipophilic amino acids (Laa) are ordered in the sequence:

(SEQ ID NOS: 88, 409-412)

Haa (Laa Laa Haa Haa)ₙ Laa), wherein n = 1-5
(Formula B). In an embodiment, n = 1 or 2.

Polypeptides described herein comprise a peptide region that is an amphipathic α helix, not merely an α-helix. Without wishing to be bound by any particular theory, the amphipathic α helix is believed to facilitate increased interaction with cell membranes and assist in proper placement of C-terminal fatty acyl chain modifications for membrane interaction. In addition, and without being bound to any particular theory, it is believed that the amphipathic helix in the C-terminal region imparts an increase in duration of action of the present polypeptides by interacting with the phospholipids of the cell membranes in the body and thereby has a “depoting” effect. Further, addition of positive charge in this amphipathic α-helical region can significantly increase the binding to the negatively charged phospholipid membrane. Such a charged region generates increased Guoy-Chapman forces that cause the peptide to accumulate on the membrane. This can be beneficial in further prolonging the duration of action and increasing the amount of peptide in the biologically active conformation in proximity to the VPAC2 receptors in the cell membranes.

Studies by Eisenberg et al. have combined a hydrophobicity scale with the helical wheel to quantify the concept of amphipathic helices (Nature 299: 371-374 (1982) and Proc. Nat. Acad. Sci. USA 81: 140-144 (1984); the disclosures of which are hereby incorporated by reference). The mean hydrophobic moment is defined as the vector sum of the hydrophobicities of the component amino acids making up the helix. The following hydrophobicities for the amino acids are those reported by Eisenberg et al. as the “consensus” scale: Ile 0.73; Phe 0.61; Val 0.54; Leu 0.53; Trp 0.37; Met 0.26 Ala 0.25; Gly 0.16; Cys 0.04; Tyr 0.02; Pro −0.07; Thr −0.18; Ser −0.26; His −0.40; Glu −0.62; Asn −0.64; Gln −0.69; Asp −0.72; Lys −1.10; Arg −1.76.

The hydrophobic moment, μH, for an ideal α-helix having 3.6 residues per turn (or a 100° arc (=360°/3.6) between side chains), may be calculated from:

μH=[(ΣH_{N} sine δ(N−1)²+(ΣH_{N} cos δ(N−1))²]^{1/2},

where H_{N} is the hydrophobicity value of the N^{th} amino acid and the sums are taken over the N amino acids in the sequence with periodicity δ=100°. The hydrophobic moment may be expressed as the mean hydrophobic moment per residue by dividing μH by N to obtain <μH>. A value of <μH> at 100°±0.20° of about 0.20 or greater is suggestive of amphipathic helix formation.

A study by Cornett et al. has further extended the study of amphiphathic α-helices by introducing the “amphipathic index” as a predictor of amphipathicity (J. Mol. Biol., 195: 659-685 (1987); the disclosure of which is hereby incorporated by reference). They concluded that approximately half of all known α-helices are amphipathic, and that the dominant frequency is 97.5° rather than 100°, with the number of residues per turn being closer to 3.7 than 3.6. The basic approach of Eisenberg, et al. is sufficient to classify a given sequence as amphipathic, particularly when one is designing a sequence ab initio to form an amphipathic α-helix.

A substitute amphipathic α-helical amino acid sequence may lack homology with the sequence of a given segment of a naturally occurring polypeptide but elicits a similar secondary structure, i.e., an α-helix having opposing polar and nonpolar faces, in the physiological environment. Replacement of the naturally occurring amino acid sequence with an alternative sequence may beneficially affect the physiological activity, stability, or other properties of the altered parent polypeptide. Exemplary reports describing the design and selection of such sequences is provided in J. L. Krstenansky, et al., FEBS Letters 242: 2, 409-413 (1989), and J. P. Segrest, et al. Proteins: Structure, Function, and Genetics 8: 103-117 (1990) among others.

Polypeptides described herein comprise amphipathic α-helix corresponding to the Formula A, wherein each Haa is independently selected from the group of hydrophilic amino acids and each Laa is independently selected from the group of lipophilic amino acids, as defined above.

In another embodiment, said residues selected from hydrophilic amino acids (Haa) and lipophilic amino acids (Laa) are ordered in the sequence of Formula B. In an embodiment, n=1 or 2.

Assuming an idealized α-helix in an embodiment of Formula A or B, wherein n=2, residues 1, 4, 5, 8, and 9 are distributed along one face (A) of the helix within about a 140° arc of each other, while residues 2, 3, 6, 7, and 10 occupy an opposing 140° arc on the other face (B) of the helix. In an embodiment, all the residues on one face are of the same polarity while all those on the other face are of the opposite polarity, i.e., if face A is all hydrophilic, face B is all lipophilic and vice versa. The skilled artisan will recognize that while the helices of the polypeptides are described by Formula A, the reverse sequence, Laa (Haa Haa Laa Laa)ₙ (hereinafter Formula A1; SEQ ID NOs:414-418) will also meet the residue distribution criteria and is an equivalent descriptor of the helices of the polypeptides described herein.

Accordingly, in another embodiment, the skilled artisan will recognize that while certain useful helices of the polypeptides are described by Formula B, the reverse sequence Laa (Haa Haa Laa Laa)ₙ Haa (hereinafter Formula B1; SEQ ID NOs: 419-423) will also meet the residue distribution criteria and is an equivalent descriptor of the helices of the described polypeptides.

Alanine may be substituted for either hydrophilic or lipophilic amino acids, since Ala can reside readily on either face of an amphipathic α-helix, although Ala-10 does not form an amphipathic α-helix. Generally, proline, cysteine, and tyrosine are not used; however, their presence and other random errors in the sequence may be tolerated (e.g., a hydrophilic residue on the lipophilic face) as long as the remaining amino acids in the segment substantially conform to the hydrophilic face—lipophilic face division. A convenient method for determining if a sequence is sufficiently amphipathic to be a sequence of this invention is to calculate the mean hydrophobic moment, as defined above. If the peak mean moment per residue at 100°±20° exceeds about 0.20, then the sequence will form an amphipathic helix and is a sequence of the invention.

In applying this concept to PACAP and VIP, it is hypothesized that either or both regions (N-terminal or C-terminal), preferably the C-terminal, may exhibit α-helical secondary structure and could be replaced with a non-homologous sequence having similar structural tendencies, without loss of biological activity or induction of immunoreaction.

It is to be fully appreciated that the provision of this amphipathic alpha-helix is one aspect of the polypeptides and uses described herein. The selection of design of other substituents, whether at the N-terminal or C-terminal, is by choice. As such, within some embodiments, there is the provision of a long acyl group at the C-terminal while, in other embodiments, it is not selected.

### Pharmaceutical Formulations

Polypeptides of the present invention may be administered in any amount to impart beneficial therapeutic effect. In a preferred embodiment, certain compounds described herein are useful in the treatment of elevated blood glucose levels, hyperglycemia, and diabetes, including Type 2 Diabetes Mellitus, insulin resistance, metabolic acidosis and obesity. In an embodiment, compounds presented herein impart beneficial activity in the modulation of insulin and/or glucose levels. In an embodiment, the present polypeptides are administered to a patient at concentrations higher or lower than that of other forms of treatment which modulate insulin and/or glucose secretion. In yet another embodiment, the present polypeptides are administered with other compounds to produce a synergistic therapeutic effect. For example, polypeptides described herein may be administered in conjunction with exendin-4 or exendin analogs.

### EXAMPLES

The following examples are provided by way of illustration only and are not intended to limit the scope of the invention.

### Example 1

### Synthetic Analogs

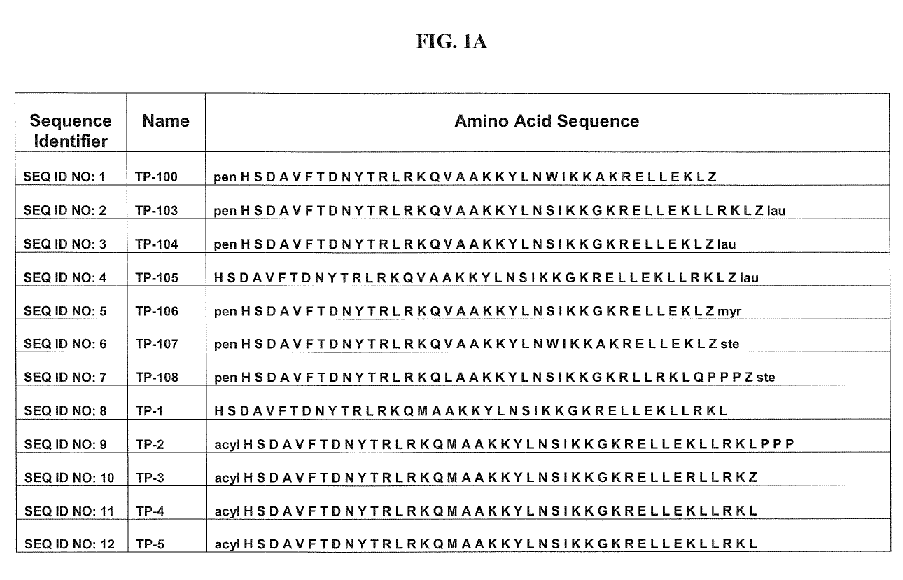

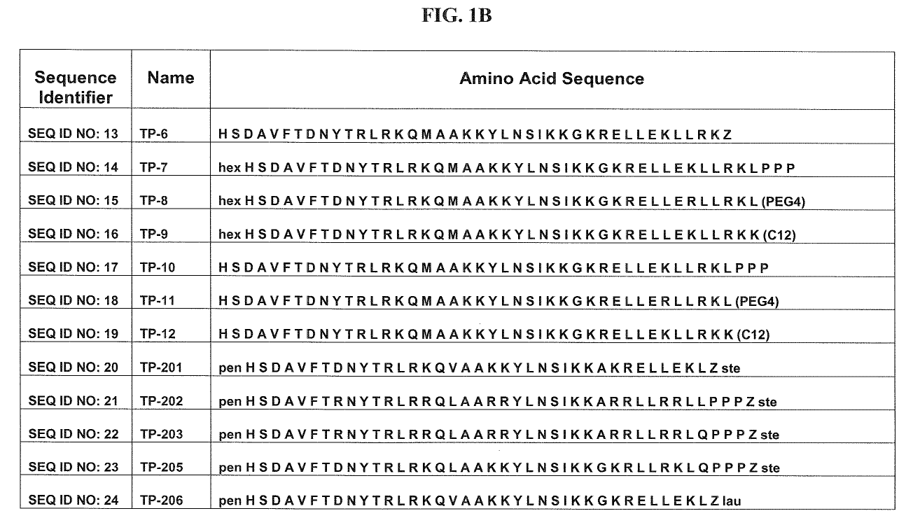

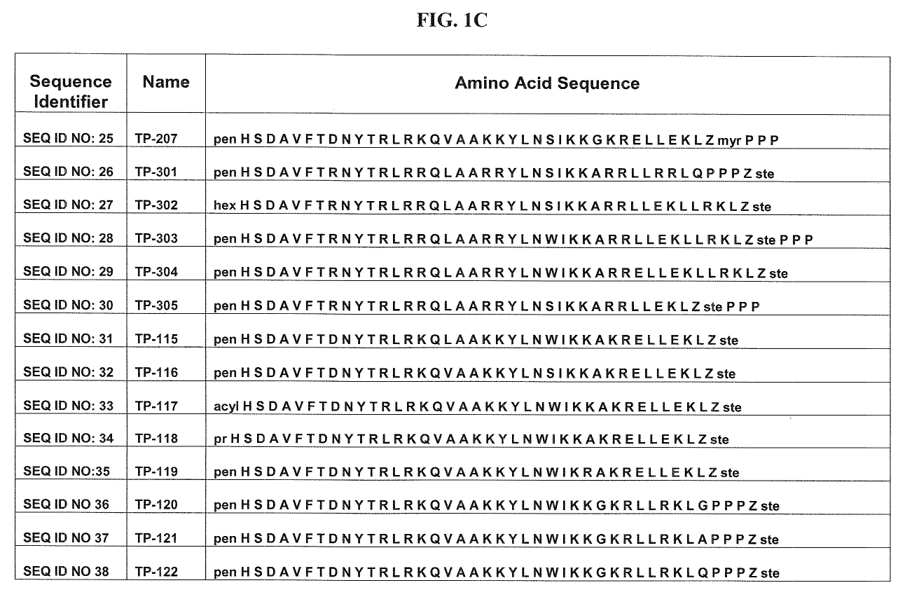

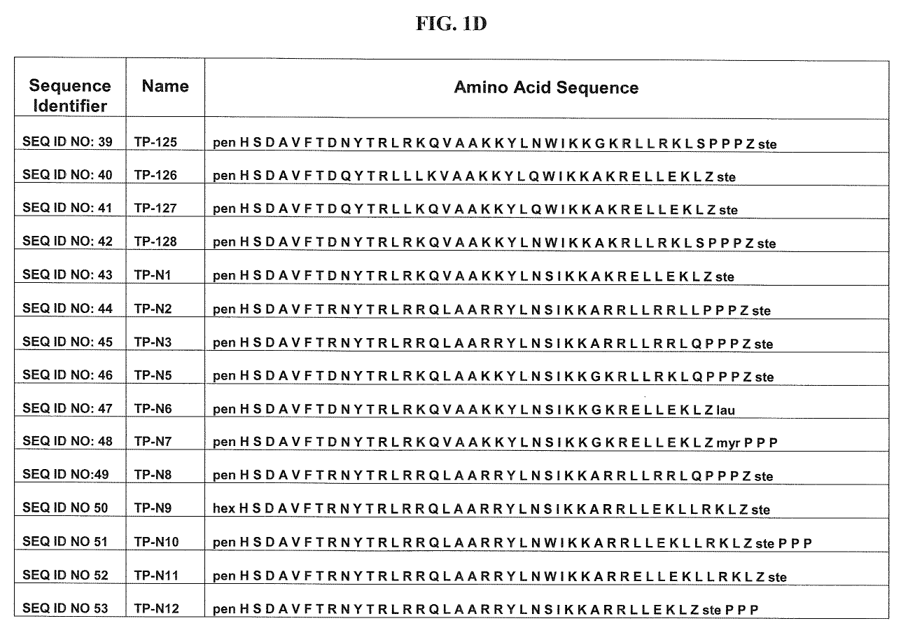

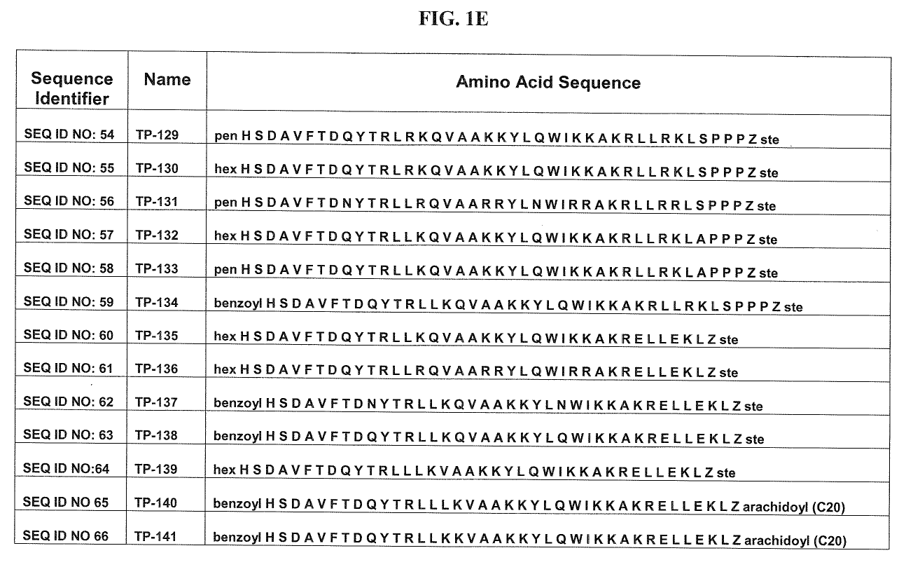

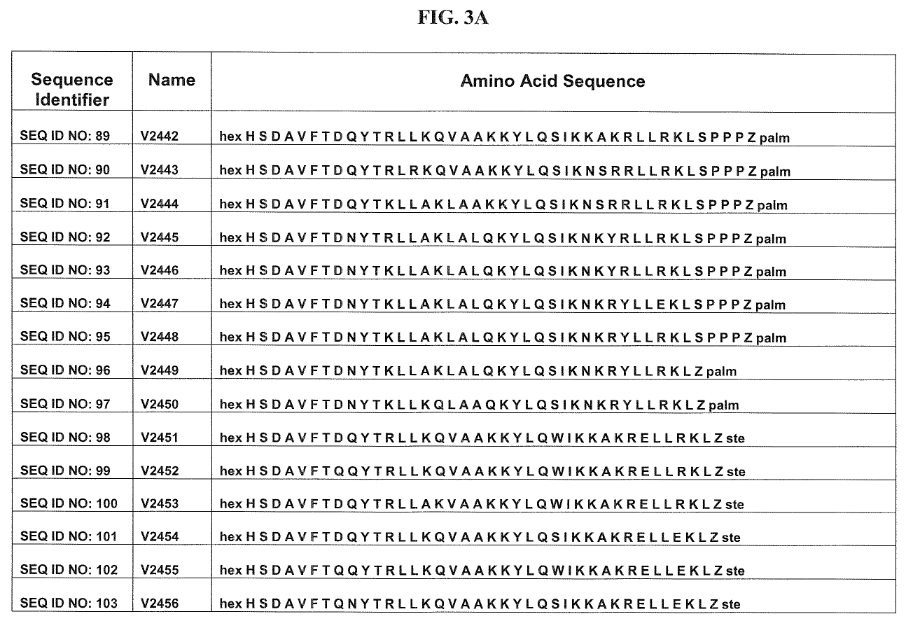

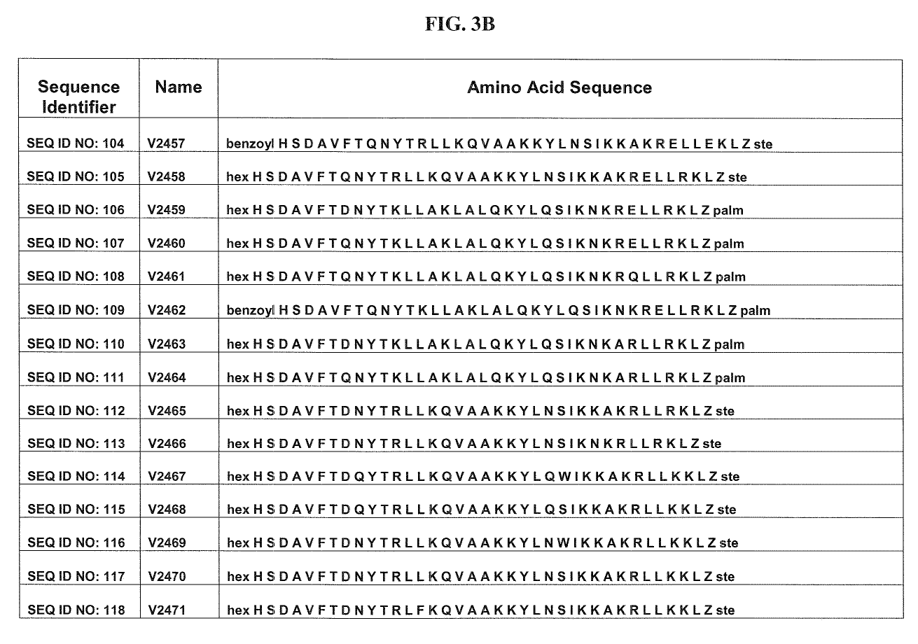

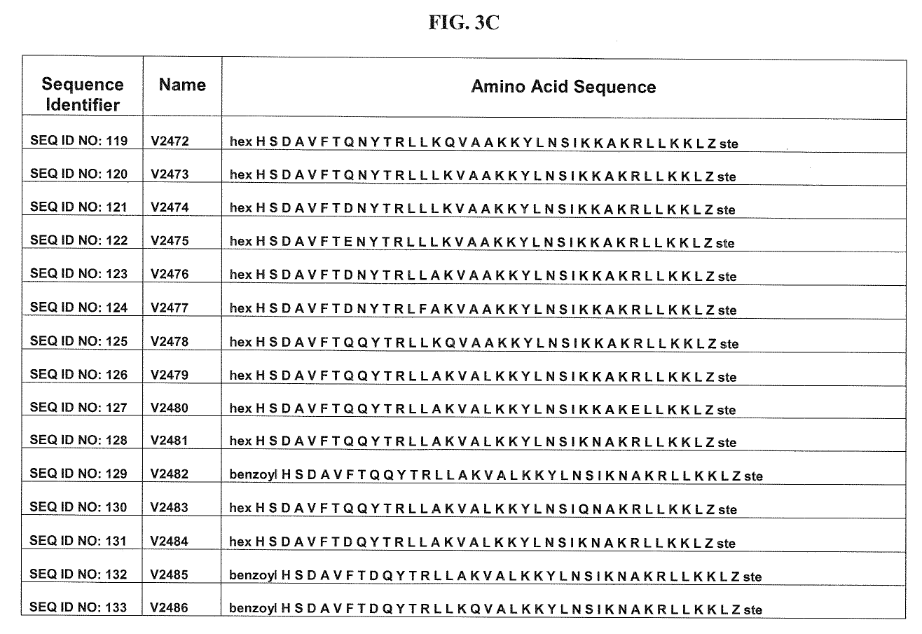

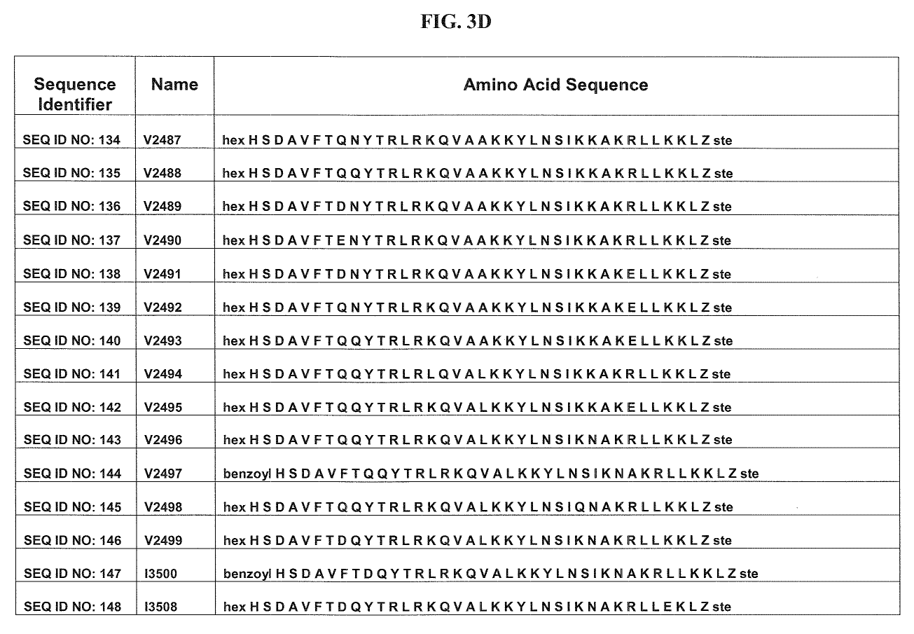

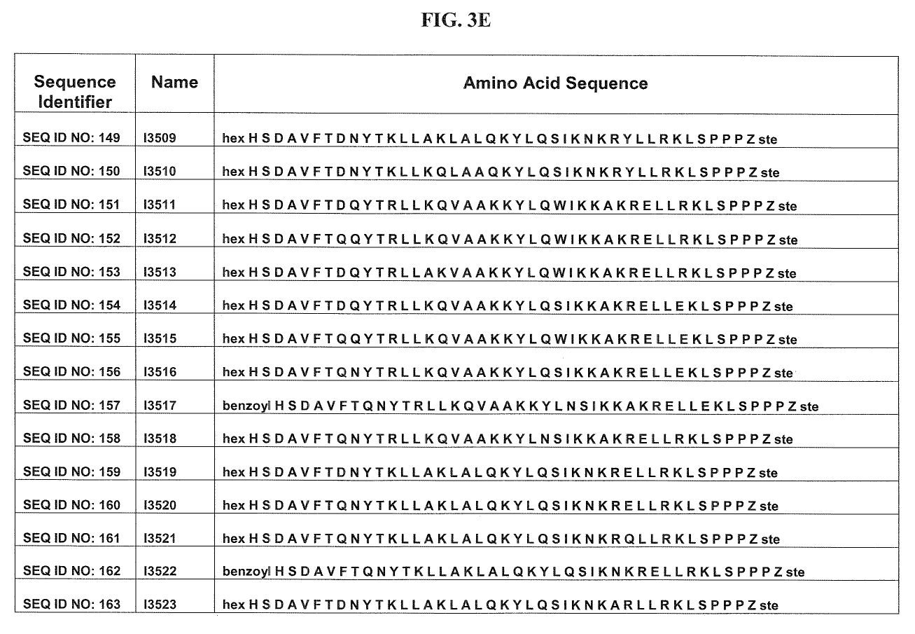

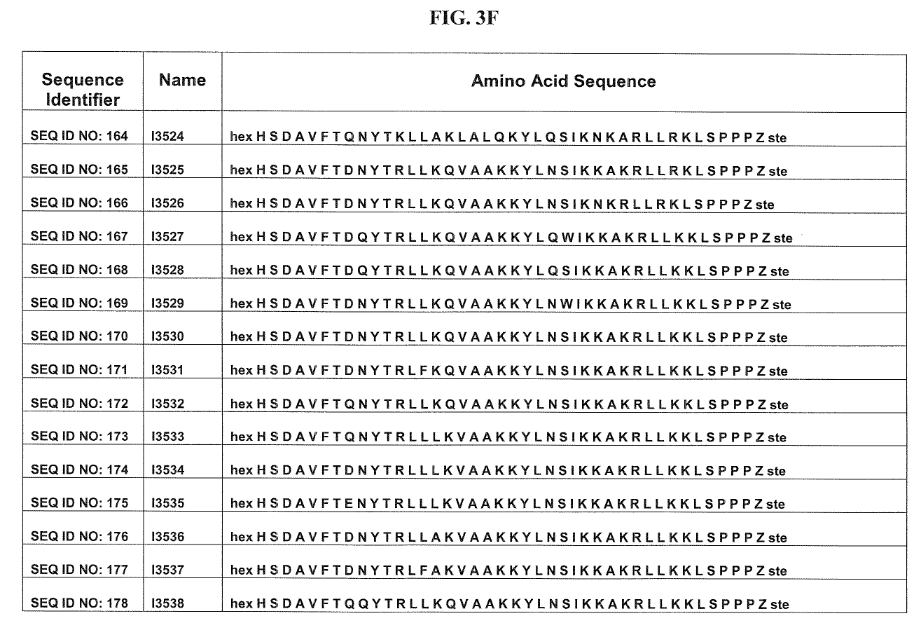

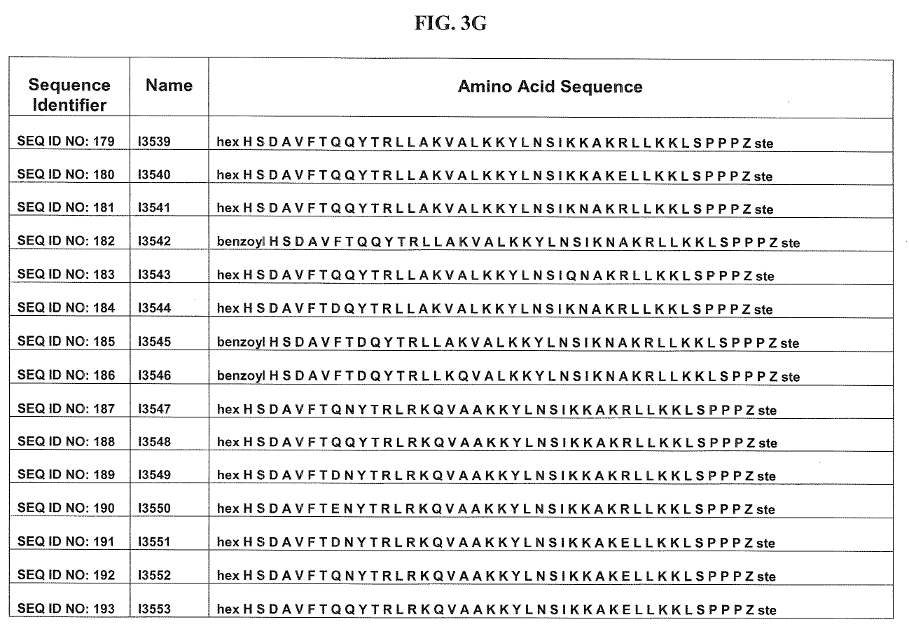

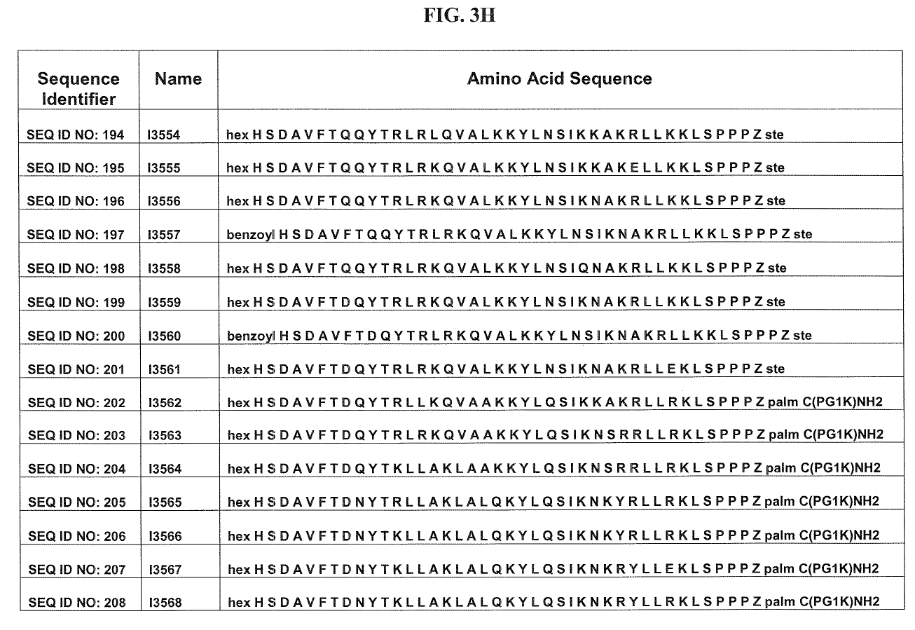

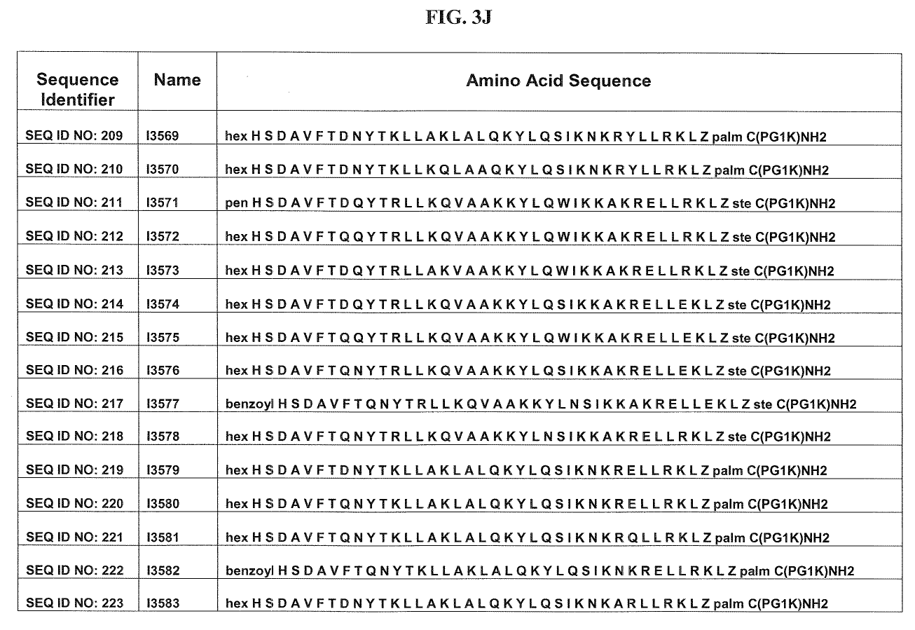

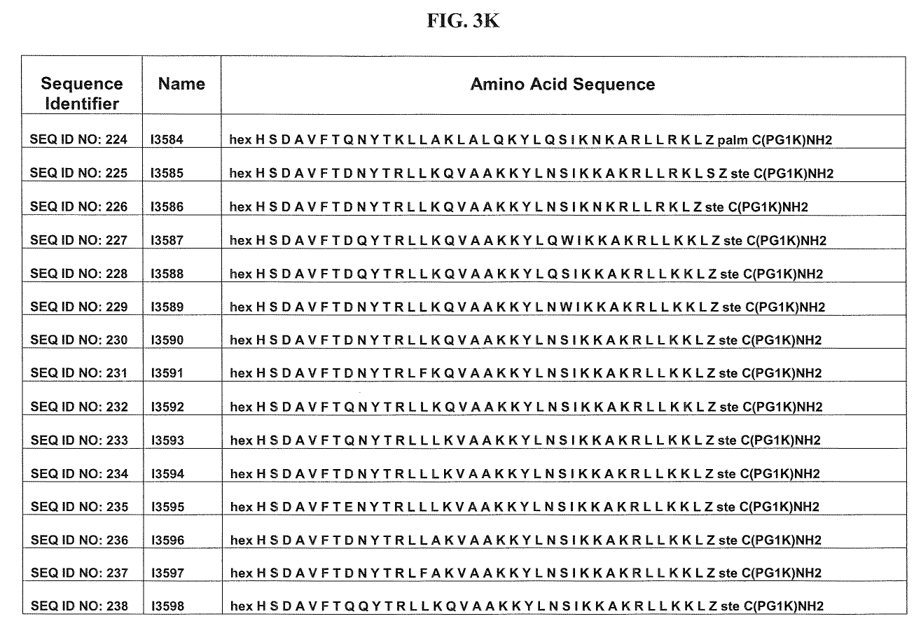

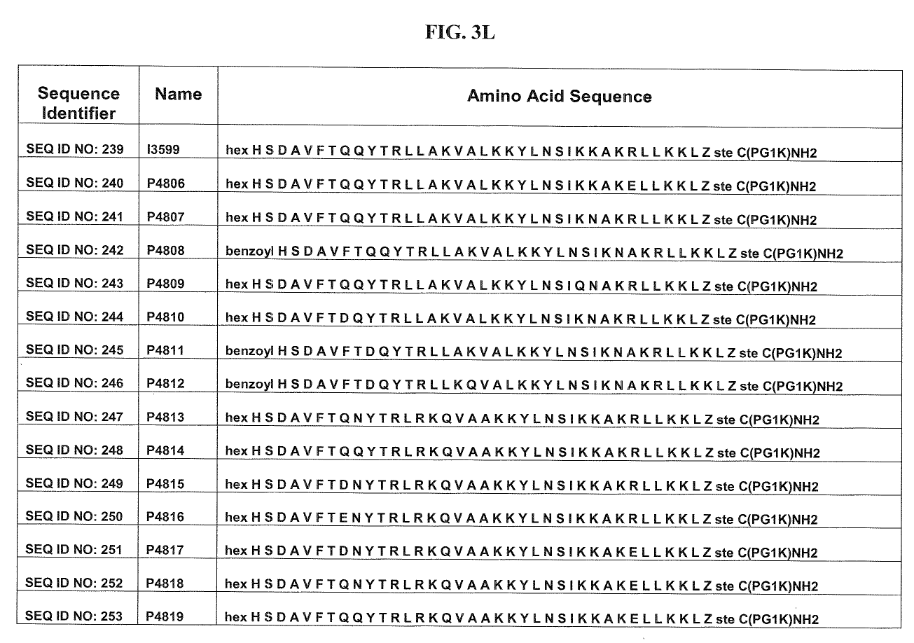

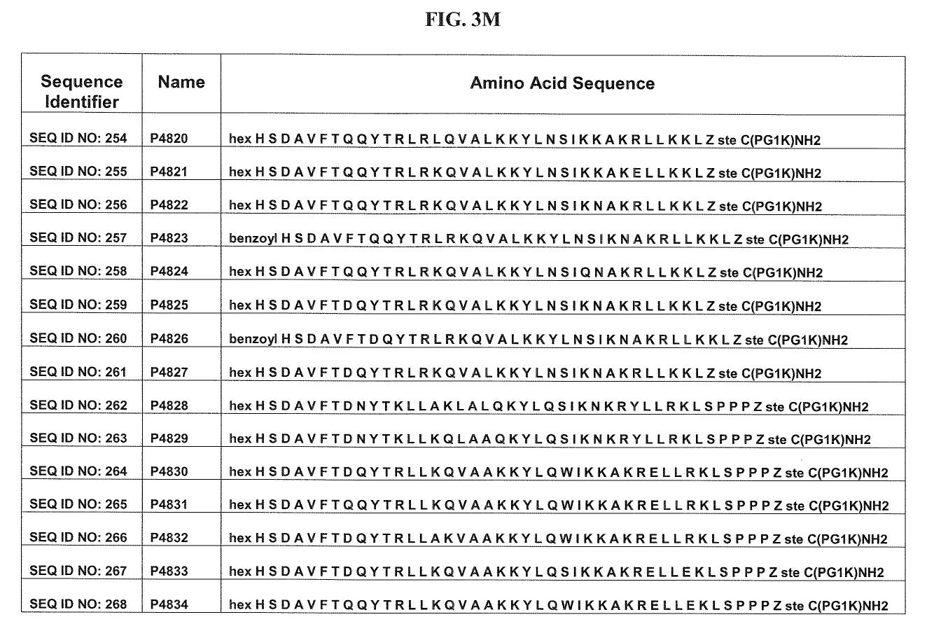

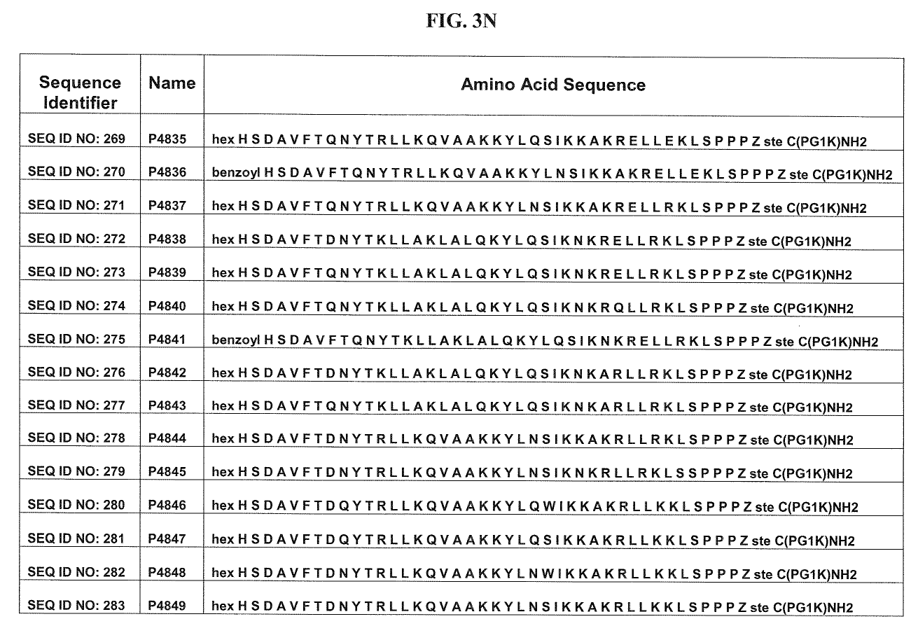

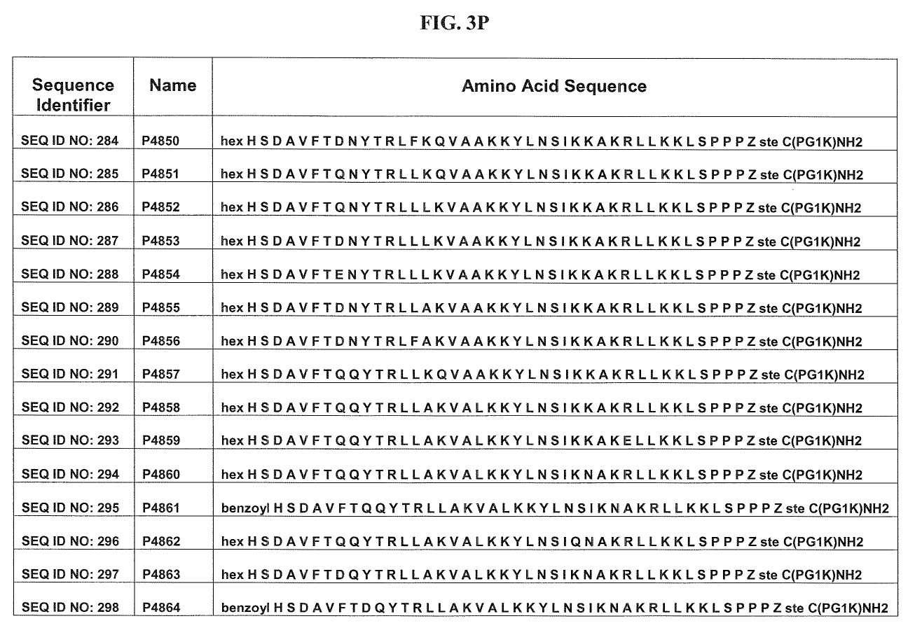

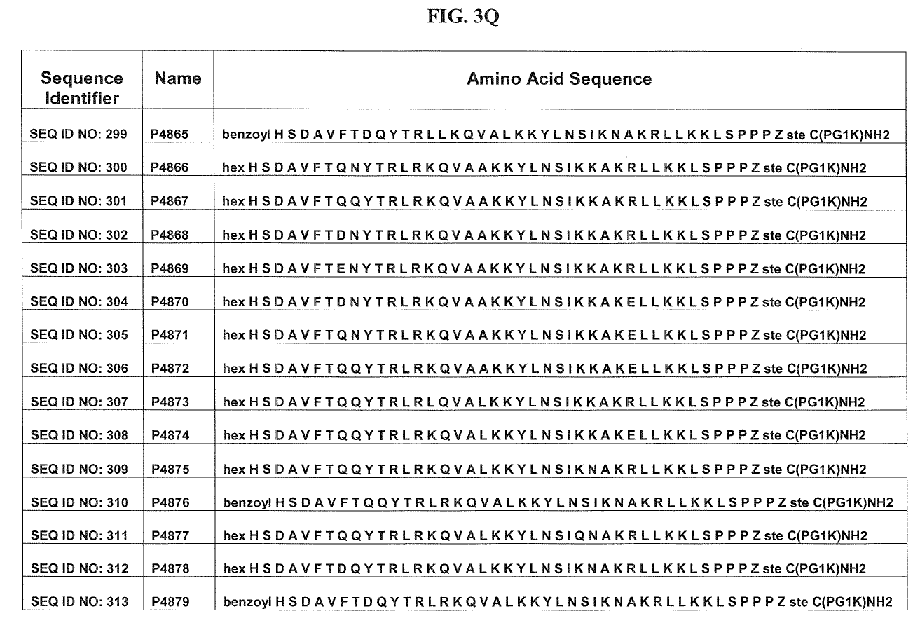

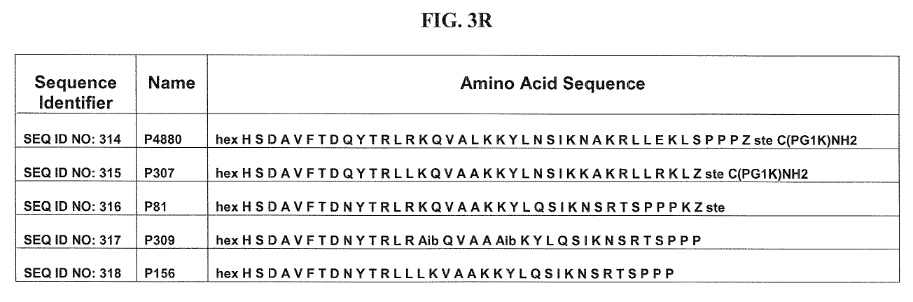

Some of the exemplary synthetic polypeptide analogs illustrated in FIGS. 1A-1E and 3A-3R are derived from VPAC2 sel UldB. Other exemplary synthetic polypeptide analogs illustrated in FIGS. 1A-1E and 3A-3R are truncated homologs of VIP.

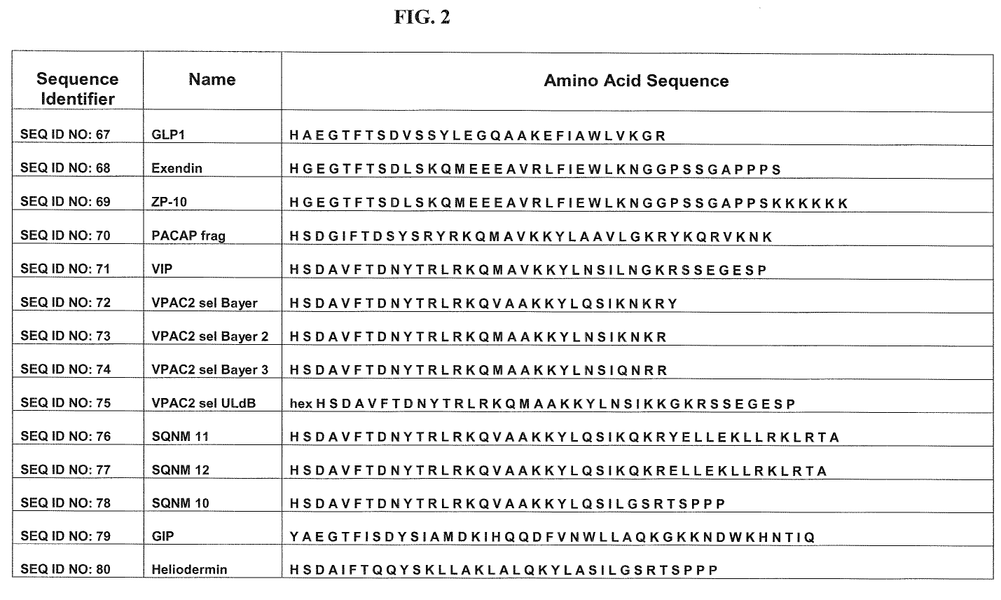

In one aspect, the present polypeptide analogs of the physiologically active truncated homologs of VIP, such as those shown in FIG. 1 as TP 1 to TP 6. Analogs TP 1 to TP 6 have a long acyl residue comprising C12-C24, preferably C16-C24. Analogs TP 7 to TP 12 shown in FIG. 1 have an acyl residue on the N-terminus comprising C₂-C₁₆, preferably C6. Analogs SQNM 10-12 (corresponding to SEQ ID NO: 76-78) shown in FIG. 2 do not contain acylation at either the C or N-termini.

Other representative polypeptide analogs presented herein have amino acid sequences of the general Formula C (SEQ ID NO: 81) with additional modifications:

Acyl-His-Ser-Asp-Xaa₄-Xaa₅-Phe-Thr-Xaa₈-Xaa₉-Tyr-
Xaa₁₁-Arg-Xaa₁₃-Xaa₁₄-Xaa₁₅-Xaa₁₆-Xaa₁₇-Ala-Xaa₁₉-
Xaa₂₀-Xaa₂₁-Tyr-Leu-Xaa₂₄-Xaa₂₅-Xaa₂₆-Xaa₂₇-Xaa₂₈-
Xaa₂₉-Xaa₃₀-Xaa₃₁-Xaa₃₂

wherein:

acyl is a C₂₋₁₆ acyl chain;

Xaa₄ is Gly or Ala;

Xaa₅ is Val, Ile, or Leu;

Xaa₈ is Asp, Arg, Gln, or Glu;

Xaa₉ is Ser, Asn, Gln, Asp or Glu;

Xaa₁₁ is Ser or Thr;

Xaa₁₃ is Leu or Tyr;

Xaa₁₄ is Arg or Leu;

Xaa₁₅ is Lys, Leu, or Arg;

Xaa₁₆ is Gln, Lys or Ala;

Xaa₁₇ is Met, Leu, Val or Ala;

Xaa₁₉ is Ala or Val;

Xaa₂₀ is Lys, Arg or Gln;

Xaa₂₁ is Lys, Arg or Gln;

Xaa₂₄ is Asn, Gln, Ala or Glu;

Xaa₂₅ is Trp, Ala, or Ser;

Xaa₂₆ is Ile, Val or Trp;

Xaa₂₇ is Leu, Lys, Arg or Gln;

Xaa₂₈ is Lys, Arg, Asn, Gln, or Gly;

Xaa₂₉ is Ala, Gly, Gln, Lys or Arg;

Xaa₃₀ is Lys, Arg, Leu, or Ala;

Xaa₃₁ is Lys, Arg, Leu, or Ala; and

Xaa₃₂ is any naturally occurring amino acid.

Formula C may be modified in various ways. For example, the C-terminal amino acid, e.g., Xaa₃₂, may be modified by the attachment of a sequence that comprises Formula A, which is further directly linked to a Lys (optionally modified on its epsilon side chain by a C₁₂₋₃₀ acyl chain), wherein the Lys is further linked to an additional moiety. That additional moiety is selected from the group consisting of OH, a Cys(PEG), a Lys(PEG) or PEG, wherein PEG is a functionalized polyethylene glycol chain of C₁₀-C₃₀₀₀ chain. The additional moiety is also selected from the formula NHR¹, wherein R¹ is selected from H, lower alkyl, haloalkyl or PEG. Further modifications of Formula C are provided by eliminating any or all of Xaa₃₀, Xaa₃₁, or Xaa₃₂. In one embodiment of the latter modifications, the next amino acid present downstream of the C-terminal amino acid is the next amino acid in the peptide agonist sequence, or the first amino acid of Formula A, which is further linked to Lys modified on its epsilon side chain by a C₁₂₋₃₀ acyl chain, wherein the Lys is further linked to an additional moiety, as described above. In a preferred embodiment, acyl is a C₂₋₈ acyl chain. In certain embodiments, Xaa₃₂ is a hydrophilic amino acid (Haa).

Other representative polypeptide analogs presented herein have amino acid sequences corresponding to general Formula C with additional modifications to those described in the paragraph above. For example, the C-terminal amino acid may be modified by the attachment of a sequence that comprises Formula A, which is further linked to an additional amino acid selected from Gln, Ser, Gly, Asp, Ala, Arg, Lys, Glu, Pro, Asn, or Leu. That additional amino acid may be subsequently followed by an optional Pro-Pro-Pro sequence, followed by the epsilon-modified Lys linked to the additional moiety as described in the paragraph above. Further modifications of these modified Formula C peptides are provided by eliminating any of Xaa₃₀, Xaa₃₁, Xaa₃₂ or the above-noted additional amino acid. In the modifications in which certain amino acids are absent, the next amino acid present downstream is the next amino acid in the peptide agonist sequence, i.e., the C-terminal amino acid is attached to Formula A, linked directly to the optional Pro-Pro-Pro sequence, followed by the epsilon-modified Lys linked to the additional moiety as described in the paragraph above. In a preferred embodiment, acyl is a C₂₋₈ acyl chain. In certain embodiments, Xaa₃₂ is a hydrophilic amino acid (Haa).

Other representative polypeptide analogs of general formula C are as follows: For example, the C-terminal amino acid may be modified by the attachment of a sequence that comprises Formula A, further linked to an additional Xaa which is Gln, Ser, Gly, Asp, Ala, Arg, Lys, Glu, Pro, Asn, or Leu, which is further linked to a Lys modified on its epsilon side chain by a C₁₂₋₃₀ acyl chain, and further linked to an additional moiety, which is PEG. In certain embodiments, PEG is a functionalized polyethylene glycol chain of C₁₀-C₃₀₀₀ chain. In certain embodiments, Xaa₃₂ is a hydrophilic amino acid (Haa). Further modifications of Formula C are provided by eliminating any or all of Xaa₃₀, Xaa₃₁, or Xaa₃₂ or the above-noted additional amino acid. In the modifications in which certain amino acids are absent the next amino acid present downstream is the next amino acid in the peptide agonist sequence or the first amino acid of Formula A directly linked to a Lys modified on its epsilon side chain by a C₁₂₋₃₀ acyl chain, wherein the Lys is further linked to an additional moiety, which is PEG. In a preferred embodiment, acyl is a C₂₋₈ acyl chain. In certain embodiments, Xaa₃₂ is a hydrophilic amino acid (Haa). The skilled artisan will appreciate that numerous permutations of the polypeptide analogs may be synthesized which will possess the desirable attributes of those described herein provided that an amino acid sequence having a mean hydrophobic moment per residue at 1000±20° greater than about 0.20 is inserted at positions in the C-terminal region.

### Example 2

### Additional Analogs

In some embodiments, representative polypeptide analogs presented herein have the following amino acid sequence of general Formula D with additional modifications:

(SEQ ID NO: 424)

Acyl-Xaa₁-Xaa₂-Xaa₃-Xaa₄-Xaa₅-Xaa₆-Thr-Xaa₈-Xaa₉₋
Xaa₁₀-Thr-Xaa₁₂-Xaa₁₃-Xaa₁₄-Xaa₁₅-Xaa₁₆-Xaa₁₇-Ala-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Xaa₃₂-Xaa₃₃-Xaa₃₄-
Xaa₃₅-Xaa₃₆-Xaa₃₇-Xaa₃₈-Xaa₃₉-Xaa₄₀

wherein:

Xaa₁ is: any naturally occurring amino acid, dH;

Xaa₂ is: any naturally occurring amino acid, dA, or dS;

Xaa₃ is: Asp or Glu;

Xaa₄ is: any naturally occurring amino acid, dA, or NMeA;

Xaa₅ is: any naturally occurring amino acid, or dV;

Xaa₆ is: any naturally occurring amino acid;

Xaa₈ is: Asp, Glu, Ala, Lys, Leu, Arg, or Tyr;

Xaa₉ is: Asn, Gln, Asp, or Glu;

Xaa₁₀ is: any naturally occurring aromatic amino acid, or Tyr (OMe);

Xaa₁₂ is: hR, Lys (isopropyl), or any naturally occurring amino acid except Pro;

Xaa₁₃ is: any naturally occurring amino acid except Pro;

Xaa₁₄ is: hR, Lys (isopropyl), or any naturally occurring amino acid except Pro;

Xaa₁₅ is: hR, Lys (isopropyl), K (Ac), or any naturally occurring amino acid except Pro;

Xaa₁₆ is: hR, Lys (isopropyl), or any naturally occurring amino acid except Pro;

Xaa₁₇ is: Nle, or any naturally occurring amino acid except Pro;

Xaa₁₉ is: any naturally occurring amino acid except Pro;

Xaa₂₀ is: hR, Lys (isopropyl), Aib, K(Ac), or any naturally occurring amino acid except Pro;

Xaa₂₁ is: hR, K(Ac), or any naturally occurring amino acid except Pro;

Xaa₂₂ is: Tyr (OMe), or any naturally occurring amino acid except Pro;

Xaa₂₃ is: any naturally occurring amino acid except Pro;

Xaa₂₄ is: any naturally occurring amino acid except Pro;

Xaa₂₅ is: any naturally occurring amino acid except Pro;

Xaa₂₆ is: any naturally occurring amino acid except Pro;

Xaa₂₇ is: hR, Lys (isopropyl), dK, or any naturally occurring amino acid except Pro;

Xaa₂₈ is: any naturally occurring amino acid, hR, dK;

Xaa₂₉ is: any naturally occurring amino acid, hR;

Xaa₃₀ is: any naturally occurring amino acid, hR; and

each of Xaa₃₁ to Xaa₄₀ is independently any naturally occurring amino acid.

Formula D may be variously modified. For example, the C-terminal amino acid may be modified by the attachment of a sequence that comprises an optional amino acid, e.g., an Xaa₄₁, linked to Formula A, wherein n=1-3, further linked to Lys (optionally modified on its epsilon side chain by a C₁₂₋₃₀ acyl chain), wherein the Lys is further linked to an additional moiety. That additional moiety is selected from the group consisting of OH, a Cys linked to a functionalized polyethylene glycol chain of C₁₀-C₃₀₀₀ chain (PEG), a LysPEG, a CysPEG and NHR¹, wherein R¹ is selected from H, lower alkyl, haloalkyl or PEG. In yet another embodiment, the C-terminal amino acid of Formula D may be modified by the attachment of a sequence that comprises an optional amino acid linked to Formula A, wherein n=1-3, further linked to an additional amino acid selected from Gln, Ser, Gly, Asp, Ala, Arg, Lys, Glu, Pro, Asn, Leu; followed by an optional Pro-Pro-Pro sequence, which is then linked to the Lys (optionally modified on its epsilon side chain by a C₁₂₋₃₀ acyl chain), wherein the Lys is further linked to the additional moiety identified above. In yet a further embodiment, Formula D is modified as immediately described above, yet lacking the optional Pro triplet, and providing that the additional moiety is a PEG. Still a further embodiment of Formula D provides that the C-terminal amino acid is linked to a polyproline type II helix. In any of these embodiments, the amino acid residues at positions 1, or 28-40, or the optional amino acid linked to Formula A or the additional amino acid linked to the C-terminus of Formula A, may be absent. In the latter circumstances, the next amino acid present downstream of the absent amino acid residue is the next amino acid in the peptide agonist sequence.

In certain embodiments, PEG is a functionalized polyethylene glycol chain of C₁₀-C₃₀₀₀ chain. In certain embodiments, PEG is a functionalized polyethylene glycol chain of C₁₀₀-C₃₀₀₀ chain. In certain embodiments, Xaa₄₁ is a hydrophilic amino acid (Haa). In some embodiments, PEG is a functionalized polyethylene glycol chain of C₁₀-C₃₀₀₀ chain. In certain embodiments, PEG is a functionalized polyethylene glycol chain of C₁₀₀-C₃₀₀₀ chain.

### Example 3

### Methods for Synthesizing Polypeptides

The polypeptides described herein may be synthesized by methods such as those set forth in J. M. Stewart and J. D. Young, Solid Phase Peptide Synthesis, 2nd ed., Pierce Chemical Co., Rockford, Ill. (1984) and J. Meienhofer, Hormonal Proteins and Peptides, Vol. 2, Academic Press, New York, (1973) for solid phase synthesis and E. Schroder and K. Lubke, The Peptides, Vol. 1, Academic Press, New York, (1965) for solution synthesis and Houben-Weyl, Synthesis of Peptides and Peptidoniinietics. 4th ed. Vol E22; M. Goodman, A. Felix, L. Moroder, C. Toniolo, Eds., Thieme: New York, 2004 for general synthesis techniques. The disclosures of the foregoing treatises are incorporated by reference herein.

Microwave assisted peptide synthesis is an attractive method and will be a particularly effective method of synthesis for the peptides described herein (Erdelyi M, et al., Synthesis 1592-6 (2002)). We have demonstrated that use of microwave-assisted synthesis has achieved large increases in purity and yield for these peptides, relative to standard synthesis techniques. For example, a typical HPLC trace was generated for a crude peptide V2449 (SEQ ID NO: 96) synthesized by standard solid phase procedures (product at retention time 14 minutes), showing the yield of pure peptide as approximately 2% from crude (data not shown). In contrast, the HPLC trace was generated for a typical microwave-assisted solid phase synthesis (product at retentional time of 26.73 min) of a VPAC2 selective analog (i.e., crude product TP-135; SEQ ID NO: 60). The yield in the latter case is 18% of pure peptide from the crude (data not shown). In other instances yields of 30% of pure peptide from crude have been achieved. Thus this method has important advantages for the synthesis of peptides of this class and size.

VIP and/or PACAP analogs, especially those described herein, are expected to have a high degree of structure due to their inherent helical preference and to the amphiphilic α-helical character designed into them. Peptides with high propensity to adopt structure in solution may be prone to synthetic difficulties due to the reduced ability of reagents to penetrate their structure and therefore reduced reactivity. The ability of microwave assistance to put energy into these chains may be of increased importance for the structures of the described polypeptides, or other VIP and/or PACAP analogs, because of their inherent helical conformational propensity. Increases in yield from 2% to roughly 20% or more can have important commercial consequences, since the former renders preparation of commercial quantities very difficult.

In further or alternative embodiments, the microwave assistance is used for synthesizing polypeptides containing at least one amino acid which is not one of the twenty standard amino acids.

Thus our process for the synthesis of VIP and/or PACAP analogs is useful for the synthesis of the compounds of the invention, but also for the synthesis of other VIP and/or PACAP analogs known in the art. Examples of the latter structures are the following owned by Eli Lilly and Co.:

(P81; SEQ ID NO: 316)

C6-HSDAVFTDNYTRLRKQVAAKKYLQSIKNSRTSPPPK(E-16)-NH₂;

(P309; SEQ ID NO: 317)

C6-HSDAVFTDNYTRLRAibQVAAAibKYLQSIKNSRTSPPP-NH₂;

(P156; SEQ ID NO: 318)

C6-HSDAVFTDNYTRLLLKVAAKKYLQSIKNSRTSPPP-NH₂.

Even if these structures do not have the amphiphilic helical character of the peptides of the invention, they are expected to have some helical potential and engender synthetic difficulties that can be remedied using the microwave-assisted synthesis techniques disclosed herein. Thus, in certain embodiments, the microwave assistance is used for synthesizing VIP and/or PACAP analogs having helical potential.

Methods for producing the polypeptide of VIP and/or PACAP analogs include synthesizing the polypeptide by the sequential addition of protected amino acids to a peptide chain, removing the protecting groups, desalting and purifying the polypeptide. In certain embodiments, the method further comprises the step of using microwave assistance. In a preferred embodiment, the method with microwave assistance produces a yield of polypepetides from about 10% to about 50%. In a more preferred embodiment, the method with microwave assistance produces a yield of polypepetides from about 12% to about 40%. In the most preferred embodiment, the method with microwave assistance produces a yield of polypepetides from about 15% to about 35%. In other embodiments, the method with microwave assistance provides a yield of polyeptides of at least two-fold increase, or between two-fold and five-fold increase as compared with a similar method without using microwave assistance.

In general, peptide synthesis methods involve the sequential addition of protected amino acids to a growing peptide chain. Normally, either the amino or carboxyl group of the first amino acid and any reactive side chain group are protected. This protected amino acid is then either attached to an inert solid support, or utilized in solution, and the next amino acid in the sequence, also suitably protected, is added under conditions amenable to formation of the amide linkage. After all the desired amino acids have been linked in the proper sequence, protecting groups and any solid support are removed to afford the crude polypeptide. The polypeptide is desalted and purified, preferably chromatographically, to yield the final product.

A preferred method of preparing the analogs of the physiologically active truncated polypeptides, having fewer than about forty amino acids, involves solid phase peptide synthesis. In this method the α-amino (Nα) functions and any reactive side chains are protected by acid- or base-sensitive groups. The protecting group should be stable to the conditions of peptide linkage formation, while being readily removable without affecting the extant polypeptide chain. Suitable α-amino protecting groups include, but are not limited to t-butoxycarbonyl (Boc), benzyloxycarbonyl (Cbz), o-chlorobenzyloxycarbonyl, biphenylisopropyloxycarbonyl, t-amyloxycarbonyl (Amoc), isobornyloxycarbonyl, α,α-dimethyl-3,5-dimethoxybenzyloxy-carbonyl, o-nitrophenylsulfenyl, 2-cyano-t-butoxycarbonyl, 9-fluorenyl-methoxycarbonyl (Fmoc) and the like, preferably Boc or more preferably, Fmoc. Suitable side chain protecting groups include, but are not limited to: acetyl, benzyl (Bzl), benzyloxymethyl (Bom), Boc, t-butyl, o-bromobenzyloxycarbonyl, t-butyl, t-butyldimethylsilyl, 2-chlorobenzyl (Cl-z), 2,6-dichlorobenzyl, cyclohexyl, cyclopentyl, isopropyl, pivalyl, tetrahydropyran-2-yl, tosyl (Tos), 2,2,4,6,7-pentamethyldihydrobenzofuran-5-sulfonyl (Pbf), trimethylsilyl and trityl. A preferred Nα-protecting group for synthesis of the compounds described herein is the Fmoc group. Preferred side chain protecting groups are O-t-Butyl group for Glu, Tyr, Thr, Asp and Ser; Boc group for Lys and Trp side chains; Pbf group for Arg; Trt group for Asn, Gln, and His. For selective modification of a Lys residue, orthogonal protection with a protecting group not removed by reagents that cleave the Fmoc or t-butyl based protecting groups is preferred. Preferred examples for modification of the Lys side chain include, but are not limited to, those removed by hydrazine but not piperidine; for example 1-(4,4-dimethyl-2,6-dioxocyclohex-1-ylidene)-3-methylbutyl (ivDde) or 1-(4,4-dimethyl-2,6-dioxocyclohex-1-ylidene)ethyl (Dde). Another orthogonal Lys side chain protecting group of use for the synthesis of the peptides described herein is the epsilon-allyloxycarbonyl (Alloc) protecting group. Selective removal in the presence of the side chain protecting groups is possible using Pd(Ph₃P)₄ based techniques as well demonstrated in the literature (for example, Kates, S. A, et al. In Peptides, Chemistry, Structure & Biology, Proc. 13th American Peptide Symposium; Hodges, R. S., Smith, J. A., Eds.; ESCOM: Leiden, 1994; Vol. 13, pp 1113-5; Gomez-Martinez, P, et al., Perkin 11: 2871-4 (1999), and references therein, all of which are incorporated herein by reference). This is particularly useful for the synthesis of the Lys side chain acylated peptides and side chain PEGylated (by acylation) peptides.

In solid phase synthesis, the C-terminal amino acid is first attached to a suitable resin support. Suitable resin supports are those materials which are inert to the reagents and reaction conditions of the stepwise condensation and deprotection reactions, as well as being insoluble in the media used. Examples of commercially available resins include styrene/divinylbenzene resins modified with a reactive group, e.g., chloromethylated co-poly-(styrene-divinylbenzene), hydroxymethylated co-poly-(styrene-divinylbenzene), and the like. Benzylated, hydroxymethylated phenylacetamidomethyl (PAM) resin is preferred for the preparation of peptide acids. When the C-terminus of the compound is an amide, a preferred resin is p-methylbenzhydrylamino-co-poly(styrene-divinyl-benzene) resin, a 2,4 dimethoxybenzhydrylamino-based resin (“Rink amide”), and the like. An especially preferred support for the synthesis of larger peptides are commercially available resins containing PEG sequences grafted onto other polymeric matricies, such as the Rink Amide-PEG and PAL-PEG-PS resins (Applied Biosystems) or similar resins designed for peptide amide synthesis using the Fmoc protocol.

Attachment to the PAM resin may be accomplished by reaction of the No protected amino acid, for example the Boc-amino acid, as its ammonium, cesium, triethylammonium, 1,5-diazabicyclo-[5.4.0]undec-5-ene, tetramethylammonium, or similar salt in ethanol, acetonitrile, N,N-dimethylformamide (DMF), and the like, preferably the cesium salt in DMF, with the resin at an elevated temperature, for example between about 40° and 60° C., preferably about 50° C., for from about 12 to 72 hours, preferably about 48 hours. This will eventually yield the peptide acid product following acid cleavage or an amide following aminolysis. The Nα-Boc-amino acid may be attached to the benzhydrylamine resin by means of, for example, an N,N′-diisopropylcarbodiimide (DIC)/1-hydroxybenzotriazole (HOBt) mediated coupling for from about 2 to about 24 hours, preferably about 2 hours at a temperature of between about 10° and 50° C., preferably 25° C. in a solvent such as dichloromethane or dimethylformamide, preferably dichloromethane.

For Boc-based protocols, the successive coupling of protected amino acids may be carried out by methods well known in the art, typically in an automated peptide synthesizer. Following neutralization with triethylamine, N,N-di-isopropylethylamine (DIEA), N-methylmorpholine (NMM), collidine, or similar base, each protected amino acid is preferably introduced in approximately 1.5 to 2.5 fold molar excess and the coupling carried out in an inert, nonaqueous, polar solvent such as dichloromethane, DMF, N-methylpyrrolidone (NMP), N,N-dimethylacetamide (DMA), or mixtures thereof, preferably in dichloromethane at ambient temperature. For Fmoc-based protocols no acid is used for deprotection but a base, preferably DIEA or NMM, is usually incorporated into the coupling mixture. Couplings are typically done in DMF, NMP, DMA or mixed solvents, preferably DMF. Representative coupling agents are N,N′-dicyclohexylcarbodiimide (DCC), N,N′-diisopropyl-carbodiimide (DIC) or other carbodiimide, either alone or in the presence of HOBt, O-acyl ureas, benzotriazol-1-yl-oxytris(pyrrolidino)phosphonium hexafluorophosphate (PyBop), N-hydroxysuccinimide, other N-hydroxyimides, or oximes. Alternatively, protected amino acid active esters (e.g. p-nitrophenyl, pentafluorophenyl and the like) or symmetrical anhydrides may be used. Preferred coupling agents are of the aminium/uronium (alternative nomenclatures used by suppliers) class such as 2-(1H-benzotriazole-1-yl)-1,1,3,3-tetramethylaminium hexafluorophosphate (HBTU), O-(7-azabenzotraiazole-1-yl)-1,1,3,3-tetramethyluronium hexafluorophosphate (HATU), 2-(6-Chloro-1H-benzotraiazole-1-yl)-1,1,3,3-tetramethylaminium hexafluorophosphate (HCTU), and the like.

A preferred method of attachment to the Fmoc-PAL-PEG-PS resin may be accomplished by deprotection of the resin linker with 20% piperidine in DMF, followed by reaction of the N-α-Fmoc protected amino acid, preferably a 5 fold molar excess of the N-α-Fmoc-amino acid, using HBTU: di-isopropylethylamine (DIEA) (1:2) in DMF in a microwave-assisted peptide synthesizer with a 5 min, 75° max coupling cycle.

For this Fmoc-based protocol in the microwave-assisted peptide synthesizer, the N-α-Fmoc amino acid protecting groups are removed with 20% piperadine in DMF containing 0.1 M 1-hydroxybenzotriazole (HOBt), in a double deprotection protocol for 30 sec and then for 3 min with a temperature maximum set at 75° C. HOBt is added to the deprotection solution to reduce aspartamide formation. Coupling of the next amino acid then employs a five fold molar excess using HBTU:DIEA (1:2) with a 5 min, 75° max. double-coupling cycle.

At the end of the solid phase synthesis the fully protected peptide is removed from the resin. When the linkage to the resin support is of the benzyl ester type, cleavage may be effected by means of aminolysis with an alkylamine or fluoroalkylamine for peptides with an alkylamide C-terminus, or by ammonolysis with, for example, ammonia/methanol or ammonia/ethanol for peptides with an unsubstituted amide C-terminus, at a temperature between about −10° and 50° C., preferably about 25° C., for between about 12 and 24 hours, preferably about 18 hours. Peptides with a hydroxy C-terminus may be cleaved by HF or other strongly acidic deprotection regimen or by saponification. Alternatively, the peptide may be removed from the resin by transesterification, e.g., with methanol, followed by aminolysis or saponification. The protected peptide may be purified by silica gel or reverse-phase HPLC.

The side chain protecting groups may be removed from the peptide by treating the aminolysis product with, for example, anhydrous liquid hydrogen fluoride in the presence of anisole or other carbonium ion scavenger, treatment with hydrogen fluoride/pyridine complex, treatment with tris(trifluoroacetyl)boron and trifluoroacetic acid, by reduction with hydrogen and palladium on carbon or polyvinylpyrrolidone, or by reduction with sodium in liquid ammonia, preferably with liquid hydrogen fluoride and anisole at a temperature between about −10° and +10° C., preferably at about 0° C., for between about 15 minutes and 2 hours, preferably about 1.5 hours.

For peptides on the benzhydrylamine type resins, the resin cleavage and deprotection steps may be combined in a single step utilizing liquid hydrogen fluoride and anisole as described above or preferably through the use of milder cleavage cocktails. For example, for the PAL-PEG-PS resin, a preferred method is through the use of a double deprotection protocol in the microwave-assisted peptide synthesizer using one of the mild cleavage cocktails known in the art, such as TFA/water/tri-iso-propylsilane/3,6-dioxa-1,8-octanedithiol (DODT) (92.5/2.5/2.5/2.5) for 18 min at 38° C. each time. Typically the fully deprotected product is precipitated and washed with cold (−70° to 4° C.) diethylether, dissolved in deionized water and lyophilized to yield the crude product as a white powder.

The peptide solution may be desalted (e.g. with BioRad AG-3® anion exchange resin) and the peptide purified by a sequence of chromatographic steps employing any or all of the following types: ion exchange on a weakly basic resin in the acetate form; hydrophobic adsorption chromatography on underivatized co-poly(styrene-divinylbenzene), e.g. Amberlite®XAD; silica gel adsorption chromatography; ion exchange chromatography on carboxymethylcellulose; partition chromatography, e.g. on Sephadex® G-25; counter-current distribution; or HPLC, especially reversed-phase HPLC on octyl- or octadecylsilylsilica (ODS) bonded phase column packing.

Thus, another aspect relates to processes for preparing polypeptides and pharmaceutically acceptable salts thereof, which processes comprise sequentially condensing protected amino acids on a suitable resin support, removing the protecting groups and resin support, and purifying the product, to afford analogs of the physiologically active truncated homologs and analogs of PACAP and VIP, preferably of PACAP and VIP in which the amino acids at the C-terminus form an amphipathic α-helical peptide sequence, as defined above.

Another aspect relates to processes for preparing polypeptides and pharmaceutically acceptable salts thereof, which processes comprise the use of microwave-assisted solid phase synthesis-based processes to sequentially condense protected amino acids on a suitable resin support, removing the protecting groups and resin support, and purifying the product, to afford analogs of the physiologically active truncated homologs and analogs of PACAP and VIP, preferably of PACAP and VIP in which the amino acids at the C-terminus form an amphipathic α-helical peptide sequence, as defined above.

### Example 4

### Exemplary Synthesis and Purification Protocol for a Representative Polypeptide Analog

Representative polypeptide analog corresponding to SEQ ID NO: 1 is prepared using the synthetic and purification methods described below.

(SEQ ID NO: 1)

Pentanoyl-His-Ser-Asp-Ala-Val-Phe-Thr-Asp-Asn-Tyr-
Thr-Arg-Leu-Arg-Lys-Gln-Val-Ala-Ala-Lys-Lys-Tyr-
Leu-Asn-Trp-Ile-Lys-Lys-Ala-Lys-Arg-Glu-Leu-Leu-
Glu-Lys-Leu-Lys(epsilon stearoyl)-NH₂

Generally, the peptide is synthesized on Fmoc-Rink-Amide-PEG resin via Fmoc chemistry. Protecting groups used for amino acid side chain functional groups are: t-Butyl group for Glu, Tyr, Thr, Asp and Ser; Boc group for Lys and Trp; Pbf group for Arg; Trt group for Asn and His. N-α-Fmoc protected amino acids are purchased from EMD Biosciences (San Diego, Calif.). Reagents for coupling and cleavage are purchased from Aldrich (St. Louis, Mo.). Solvents are purchased from Fisher Scientific (Fairlawn, N.J.).

Generally, the synthetic protocol involved assembly of the peptide chain on resin by repetitive removal of the Fmoc protecting group and coupling of protected amino acid. For the synthesis, Dde-Lys(Fmoc)-OH is coupled onto the deprotected Rink Amide resin first. The side chain Fmoc protecting group is then removed by 20% piperidine in DMF. Stearic acid is coupled onto the side chain of Lys using HBTU, HOBt and NMM. The Dde group is removed by 2% hydrazine in DMF and the next Fmoc protected amino acid is coupled. HBTU and HOBt are used as coupling reagent and NMM is used as base. After removal of last Fmoc protecting group, valeric acid (4 equivalents) is coupled to the amino terminus with DIC (4 equivalents) and HOBt (4 equivalents). The peptide resin is treated with cocktail 1 for cleavage and removal of the side chain protecting groups. Crude peptide is precipitated from cold ether and collected by filtration.

An alternative method for incorporation of the C-terminal Lys side chain modification is to use Nα-Fmoc-Lys(ivDde) at the C-terminus and remove the ivDde with triple deprotection with 2% hydrazine/DMF prior to coupling with stearic acid or other modifying reagent. Final cleavage and deprotection then ensues. Another preferred method entails the use of Nα-Fmoc-Lys(Alloc) at the C-terminus. Following the building of the chain, the Alloc group is removed using Pd(0)PPh₃ and one of various scavenging agents known in the art as outlined above (especially phenylsilane or aminoborane conjugates, per publications by Gomez-Martinez, P and earlier by Albericio, F.). Again, the deprotected C-terminal Lys sidechain is reacted with a modifying agent like stearic acid. In this discussion, “C-terminal Lys” means a residue near the C-terminus, as some constructions have a Lys-Cys-NH₂ or similar construction at the C-terminus.

Purification of crude peptide is achieved via RP-HPLC using 20 mm×250 mm column from Waters (Milford, Mass.). Peptide is purified using TFA Buffer. A linear gradient of 35% to 55% acetonitrile in 60 minutes is used. Pooled fractions are lyophilized. The peptide identity is verified by mass spectrometry analysis and amino acid analysis. The peptide purity is determined by analytical HPLC column (C18 column, 4.6×250 mm, manufactured by Supelco (St. Louis, Mo.)) chromatography.

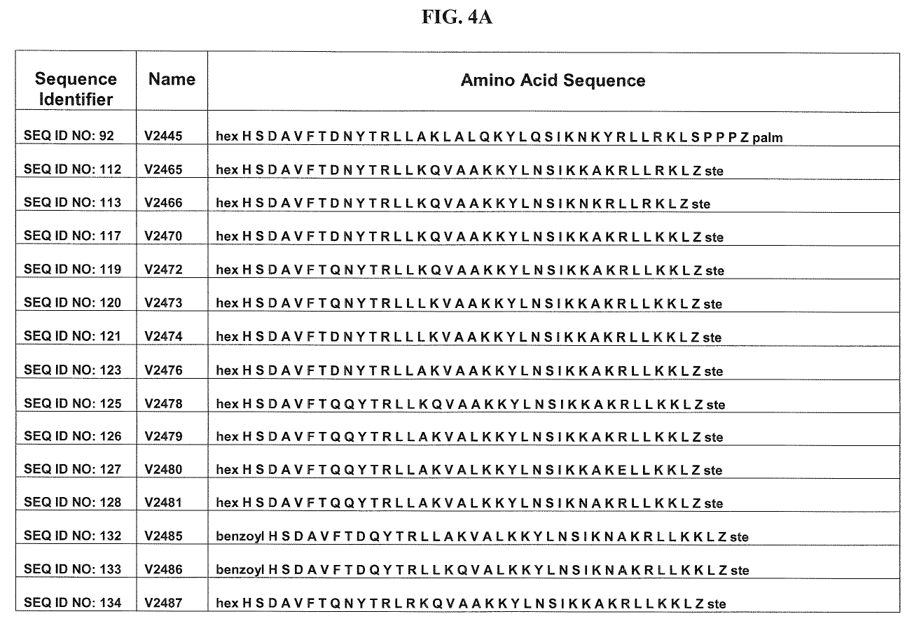

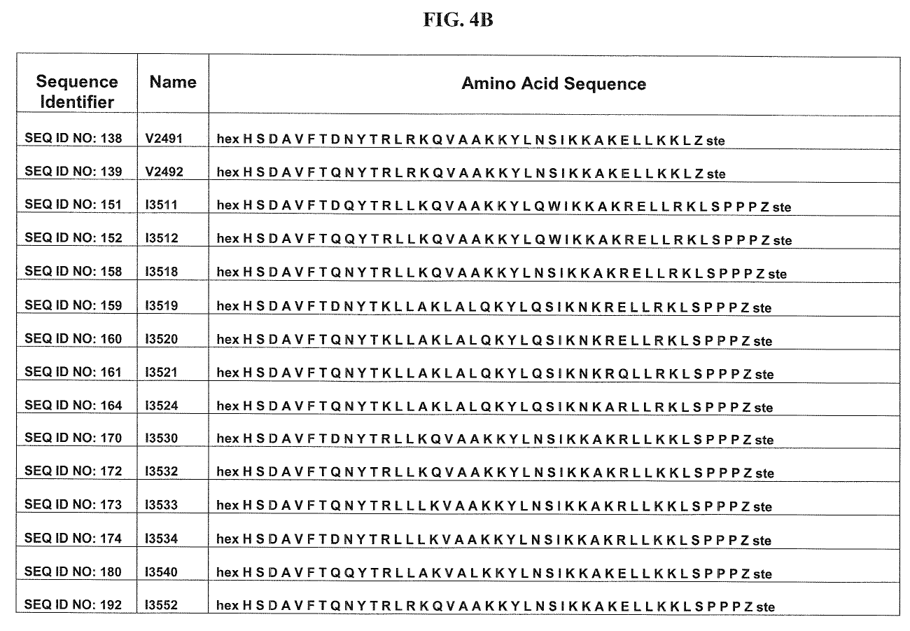

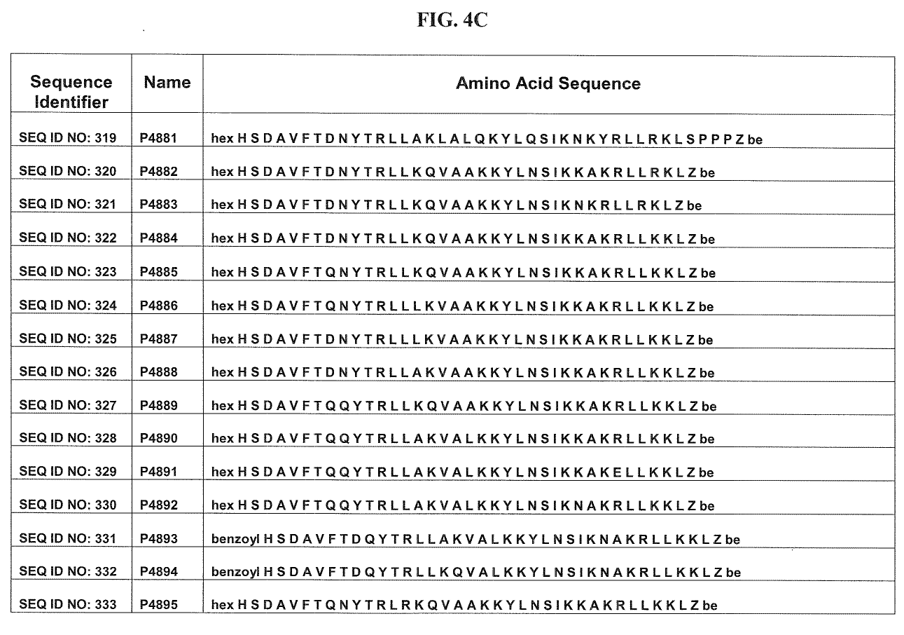

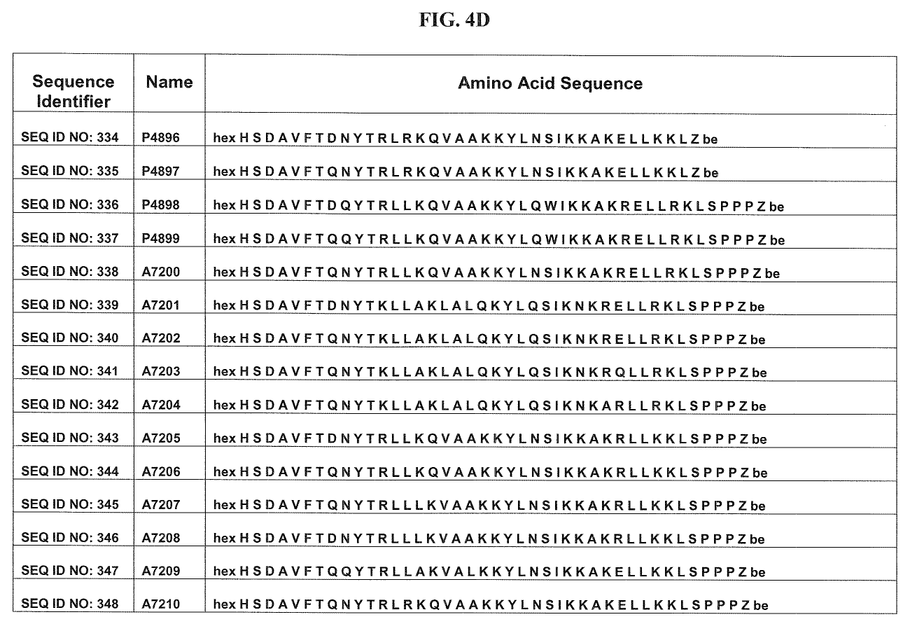

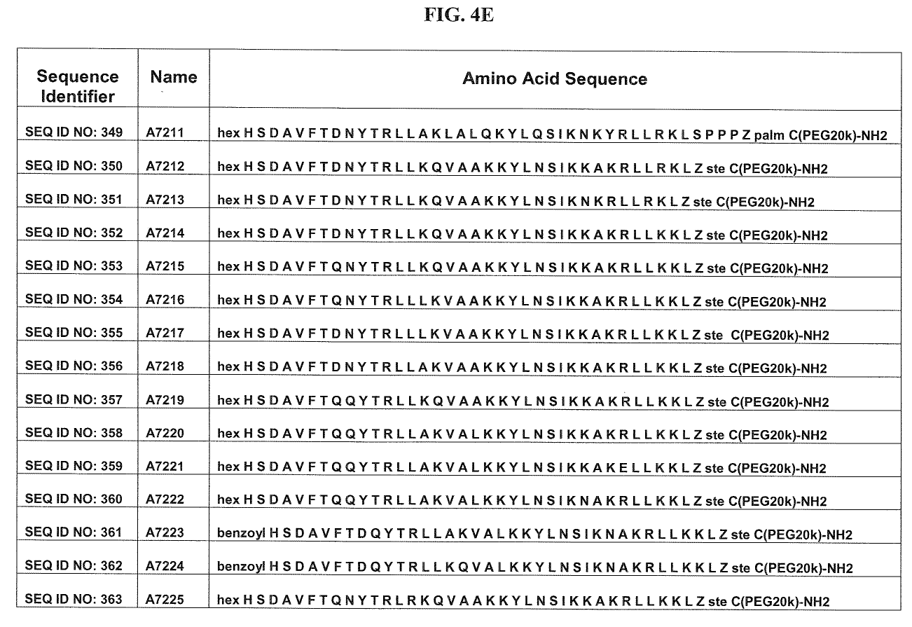

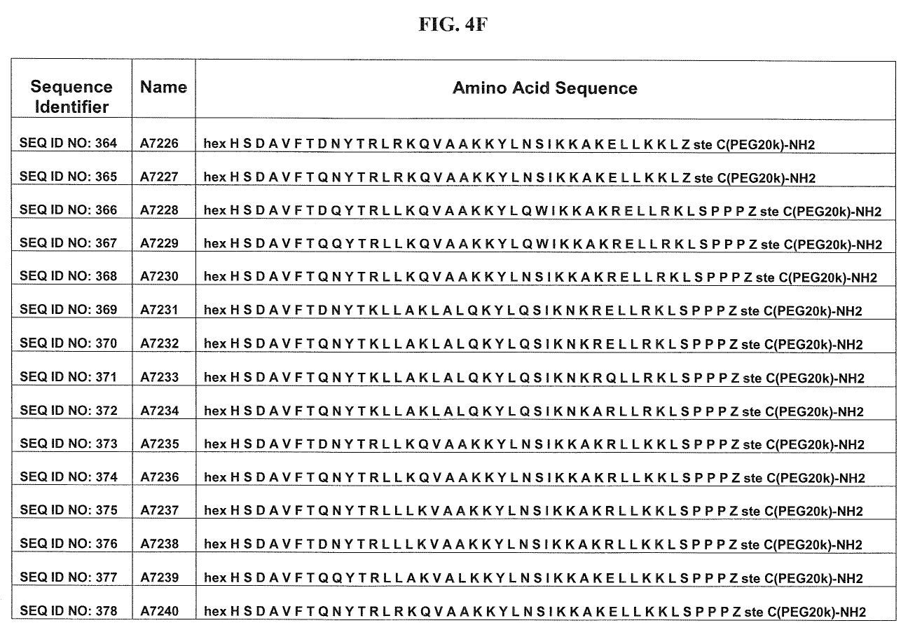

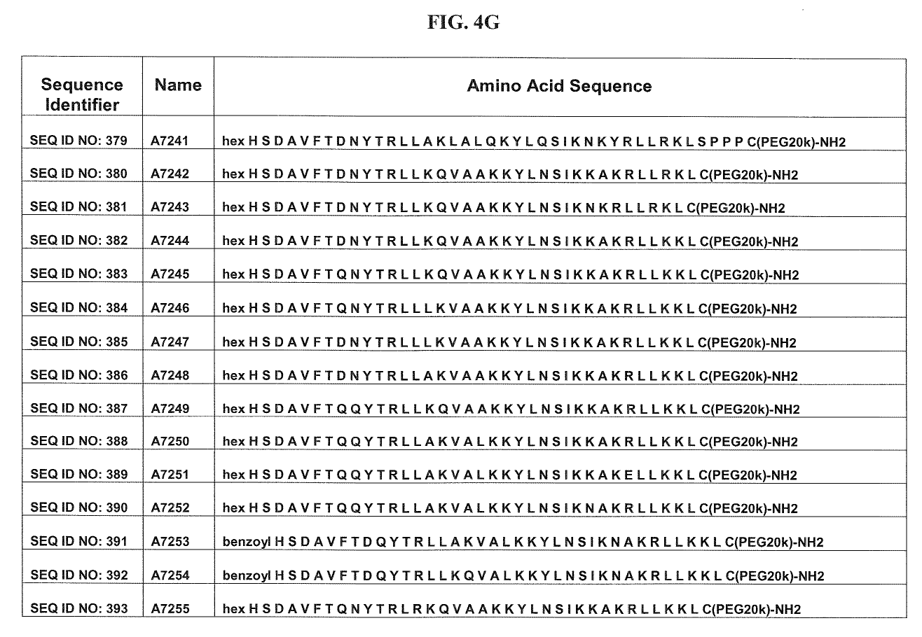

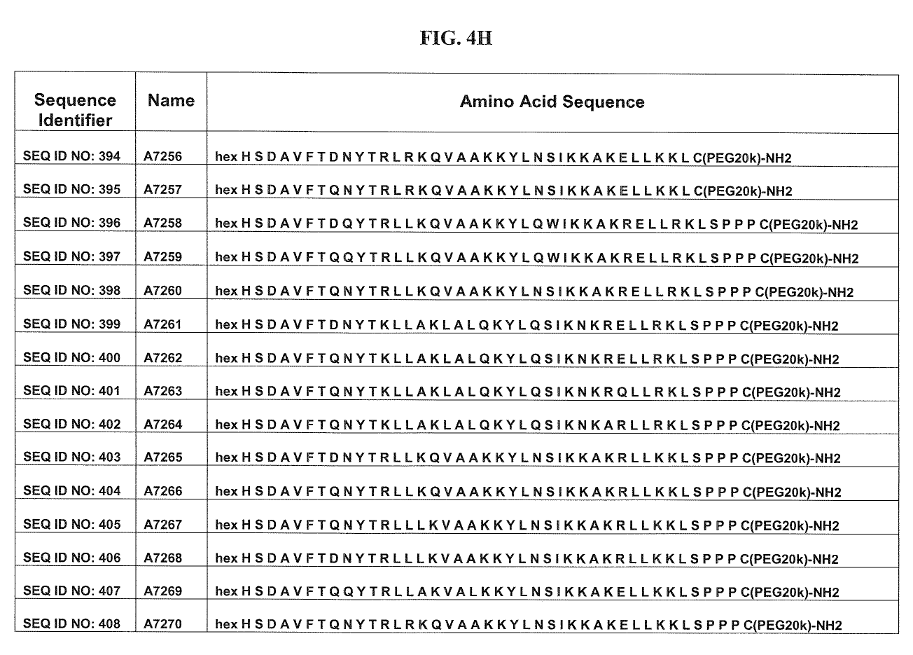

The above procedure can be summarized in the following step wise protocol:
Step 1. Resin swelling: Fmoc-Rink-Amide-PEG resin is swelled in DCM for 30 minutes (10 ml/g resin)
Step 2. Deprotection:
   a. 20% piperidine/DMF solution (10 ml/g resin) is added to the resin;
   b. Solution stirred for 30 minutes (timing is started when all the resin is free floating in the reaction vessel); and
   c. Solution is drained.
Step 3. Washing: Resin is washed with DMF (10 ml/g resin) five times. The ninhydrin test is performed and appeared positive.
Step 4. Coupling:
   a. Fmoc-AA-OH (3 equivalents calculated relative to resin loading) and HOBt (3 equivalents relative to resin loading) is weighed into a plastic bottle.
   b. Solids are dissolved with DMF (5 ml/g resin).
   c. HBTU (3 equivalents relative to resin loading) is added to the mixture, followed by the addition of NMM (6 equivalents relative to resin loading).
   d. Mixture is added to the resin.
   e. Mixture is bubbled (or stirred) gently for 10-60 minutes until a negative ninhydrin test on a small sample of resin is obtained.
Step 5. Washing: Resin is washed three times with DMF.
Step 6. Steps 2-5 are repeated until the peptide is assembled.
Step 7. N-terminal Fmoc Deprotection: Step 2 is repeated.
Step 8. Washing and Drying:
   a. After the final coupling, resin is washed three times with DMF, one time with MeOH, three times with DCM, and three times with MeOH.
   b. Resin is dried under vacuum (e.g., water aspirator) for 2 hours and high vacuum (oil pump) for a minimum of 12 hours.
Step 9. Cleavage:
   a. Dry resin is placed in a plastic bottle and the cleavage cocktail is added. The mixture is shaken at room temperature for 2.5 hours.
   b. The resin is removed by filtration under reduced pressure. The resin is washed twice with TFA. Filtrates are combined and an 8-10 fold volume of cold ether is added to obtain a precipitate.
   c. Crude peptides are isolated by filtration and then washed twice with cold ether. FIG. 4 shows an HPLC trace of a typical crude peptide which typically yields purified peptide on scale of 5% or less from the crude material.

The following chemicals and solvents are used in the synthetic protocol described above: NMM (N-Methylmorpholine); HBTU (2-(1H-Benzotriazole-1-yl)-1,1,3,3-tetramethyluronium Hexafluorophosphate); HOBt (1-Hydroxybenzotriazole); DMF (Dimethylformamide); DCM (Dichloromethane); Methanol; Diethylether; Piperidine; T is (Triisopropylsilane); Thioanisole; Phenol; EDT (1,2-Ethanedithiol); Trifluoroacetic acid Cocktail 1: TFA/Thioanisole/Phenol/H2O/EDT (87.5/5/2.5/2.5/2.5 v/v/); TFA buffer: A (0.1% TFA in water); and TFA buffer B (0.1% TFA in Acetonitrile).

Other representative polypeptide analogs are prepared in a manner similar to that described above. Listed below in TABLE 1 are chemical properties of exemplary polypeptide analogs described herein.

TABLE 1
Properties of Exemplary Polypeptide Analogs

| Name | | Purity Based on | Molecular Weight Based on |
|---|---|---|---|
| of | Amino Acid | RP-HPLC | Electrospray Mass |
| Analog | Sequence | Chromatogram | Spectrometry |
| TP-103 | SEQ ID NO: 2 | 96.9% | 5267.2 a.m.u. |
| TP-104 | SEQ ID NO: 3 | 95.5% | 4756.7 a.m.u. |
| TP-105 | SEQ ID NO: 4 | 96.1% | 5183.3 a.m.u. |
| TP-106 | SEQ ID NO: 5 | 95.2% | 4784.8 a.m.u. |
| TP-107 | SEQ ID NO: 6 | 99.6% | 4955.1 a.m.u. |
| TP-108 | SEQ ID NO: 7 | 91.5% | 5172.4 a.m.u. |

### Example 5

### Exemplary Microwave-Assisted Synthesis and Purification Protocol for a Representative Polypeptide Analog

Representative polypeptide analog corresponding to SEQ ID NO: 60 (TP-135) is prepared using the synthesis and purification methods described below.

(SEQ ID NO: 60)

Hexanoyl-His-Ser-Asp-Ala-Val-Phe-Thr-Asp-GLn-Tyr-
Thr-Arg-Leu-Leu-Lys-Gln-Val-Ala-Ala-Lys-Lys-Tyr-
Leu-Gln-Trp-Ile-Lys-Lys-Ala-Lys-Arg-Glu-Leu-Leu-
Glu-Lys-Leu-Lys(stearoyl)-NH₂

Generally, the peptide is synthesized on a CEM Liberty Automated Peptide

Synthesizer on 0.1 mmol scale. This synthesizer uses microwave-assisted synthesis and has the ability to monitor internal reaction vessel temperatures. Fmoc-PAL-PEG-PS resin (0.18 mmol/gm nominal substitution) is used as support with N-α-Fmoc protecting group chemistry. Protecting groups used for amino acid side chain functional groups are: O-t-Butyl group for Glu, Tyr, Thr, Asp and Ser; Boc group for Lys and Trp side chains, except for the C-terminal Lys; Pbf group for Arg; Trt group for Asn, Gln, and His. Reagents for coupling and cleavage, as well as N-α Fmoc protected amino acids, are from CEM Corporation (Matthews, N.C.). N-α-Fmoc deprotection is carried out with 20% piperidine in DMF containing 0.1M HOBt. Double Fmoc deprotection is carried out for 30 sec and then for 3 min with a temperature maximum set at 75° C. For the removal of side chain ivDde protection from the C-terminal Lys residue, a triple deprotection scheme with 2% hydrazine in DMF is used: 3 min/6 min/6 min, 75° C. max. Amino acid activation is carried out on five fold molar excess using HBTU:DIEA (1:2) with a 5 min, 75° max. double-coupling cycle on all residues, except single coupling on Fmoc-Lys(ivDde)-OH (initial step) and triple coupling of stearic acid (final assembly step).

The synthetic protocol generally involves assembly of the peptide chain on resin by repetitive removal of the Fmoc protecting group and coupling of protected amino acid, similar to that described in example 4 above, but with differences in side chain protection, molar excess, etc. as described herein. For the synthesis, Fmoc-Lys(ivDde)-OH is coupled onto the deprotected, commercially available Fmoc-PAL-PEG-PS resin first. The Fmoc protecting group is then removed by 20% piperidine in DMF. The peptide is assembled by repetitive cycles of coupling, Fmoc deprotection and further coupling. Following the last amino acid coupling, the N-α-Fmoc group is removed from His(Trt) and it is coupled with hexanoic acid (double coupling protocol). At this point, preferably approximately one half of the peptide resin is removed and saved for other analog syntheses.

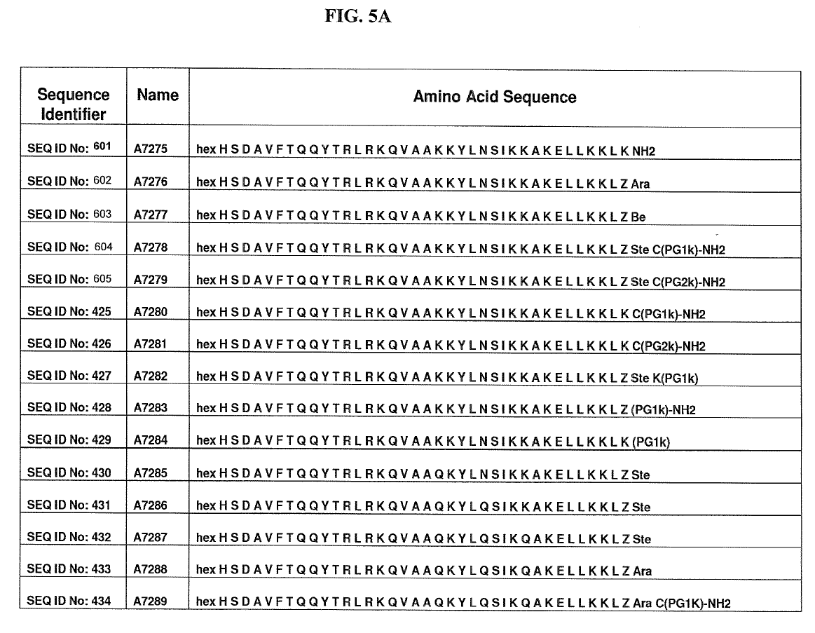

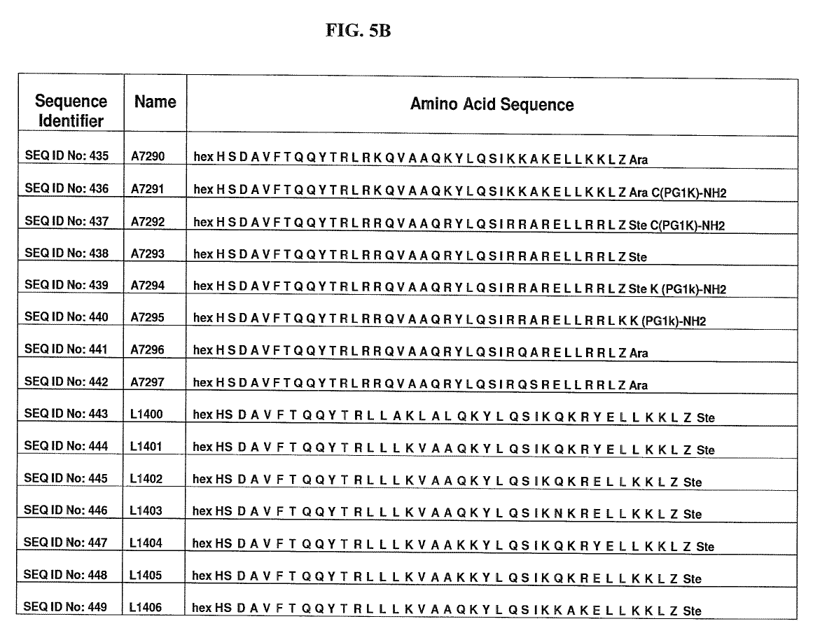

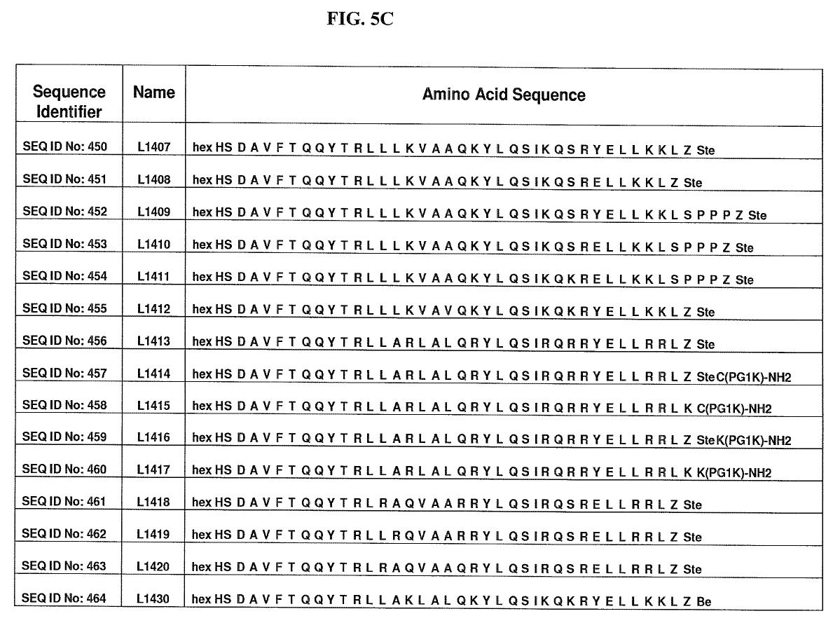

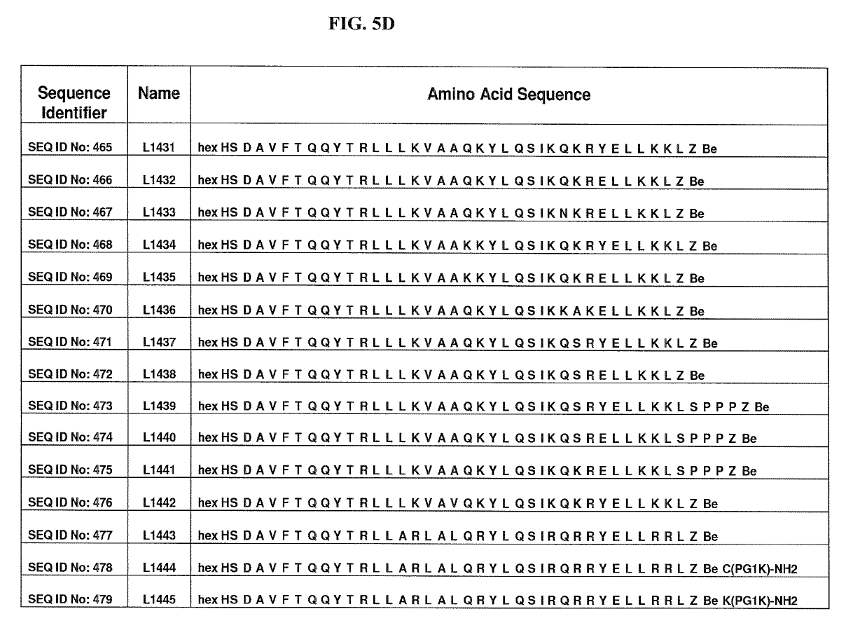

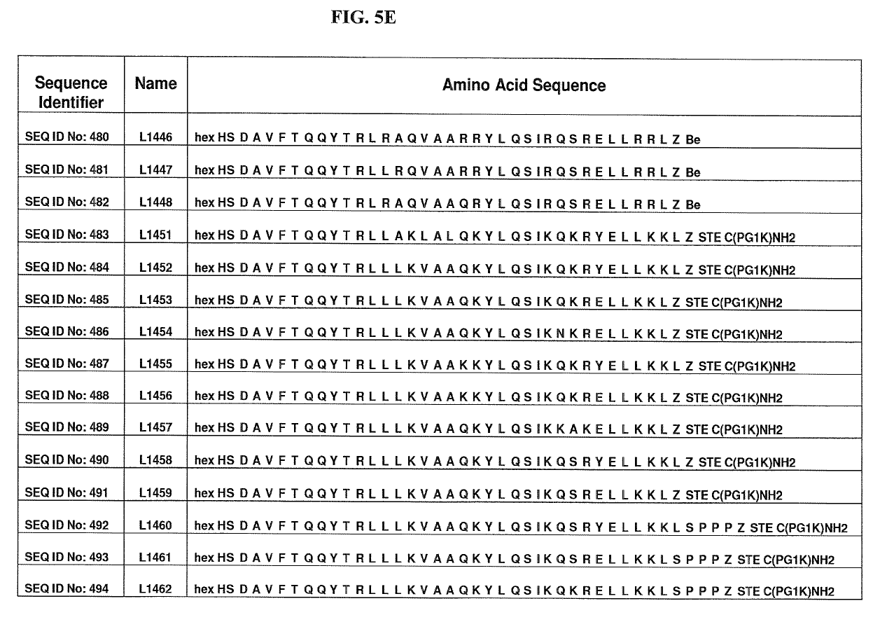

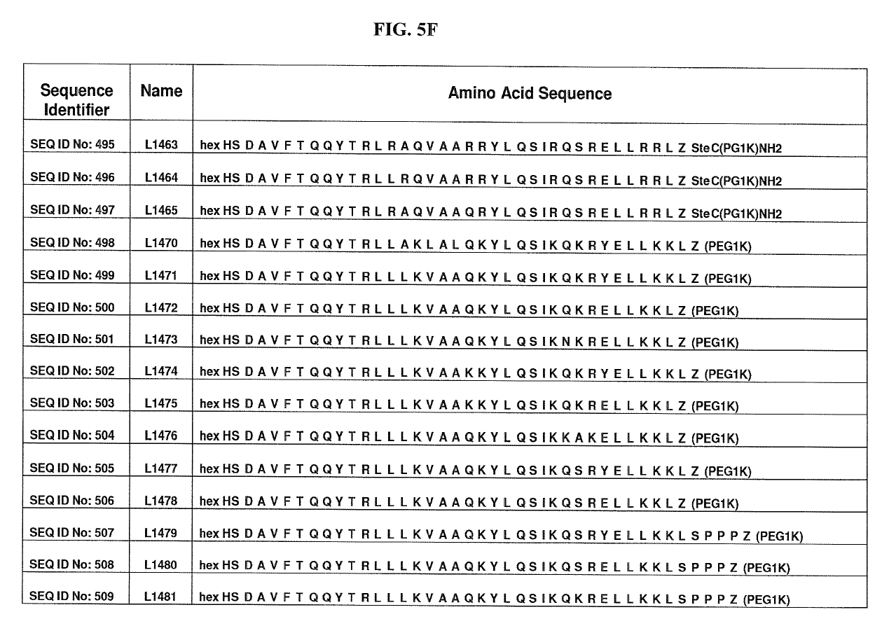

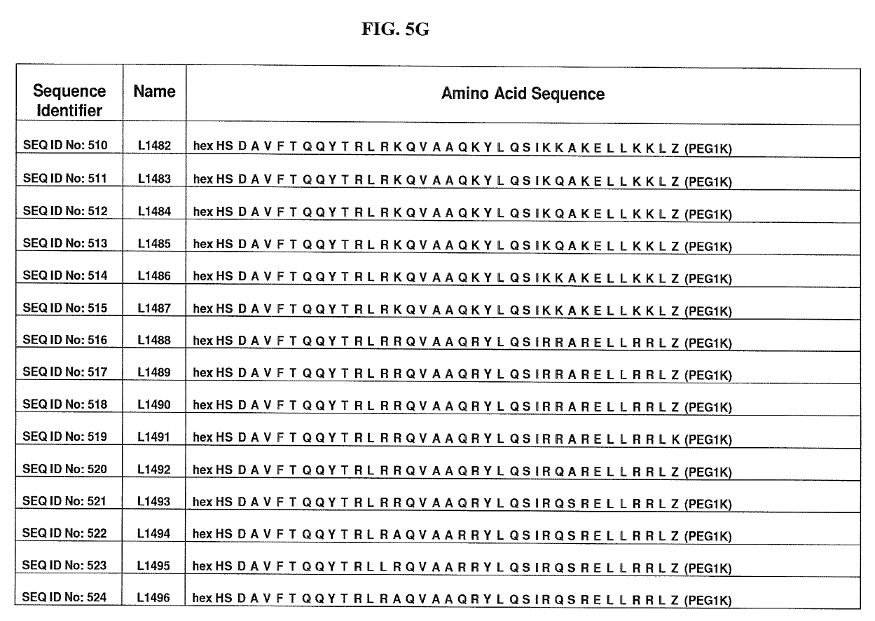

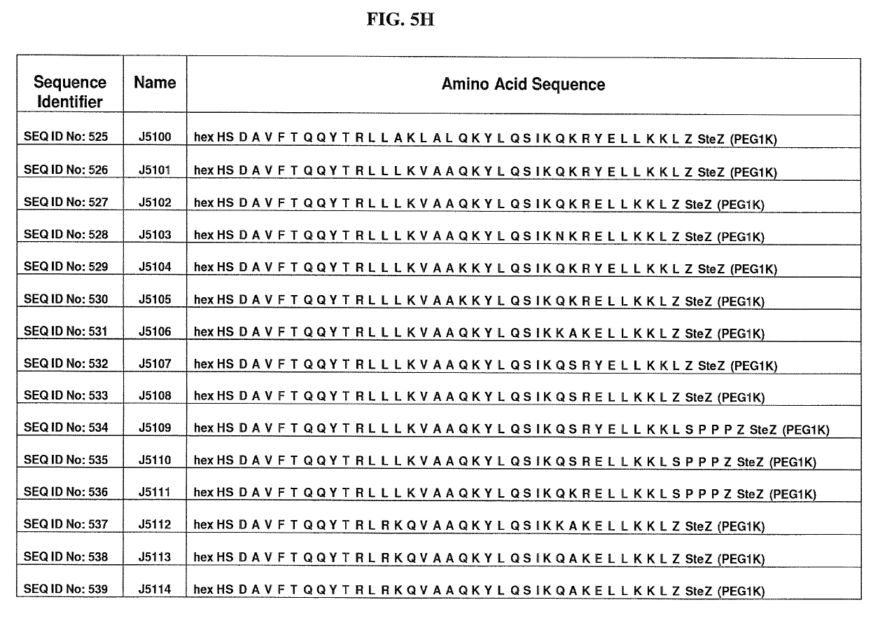

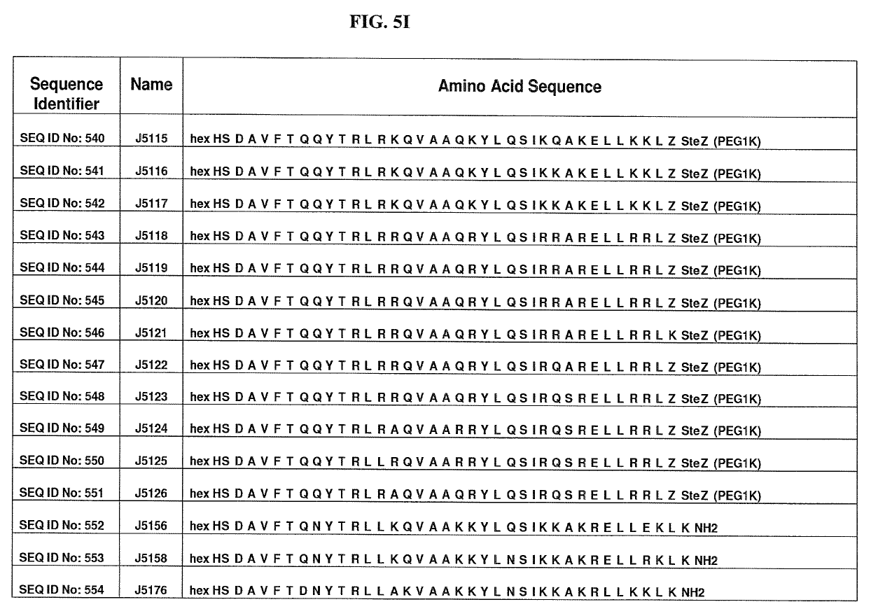

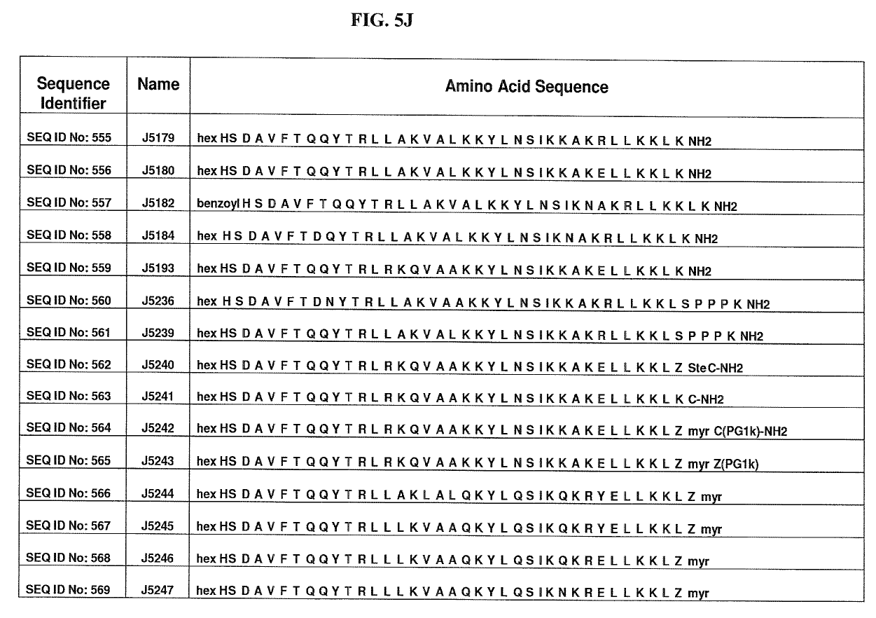

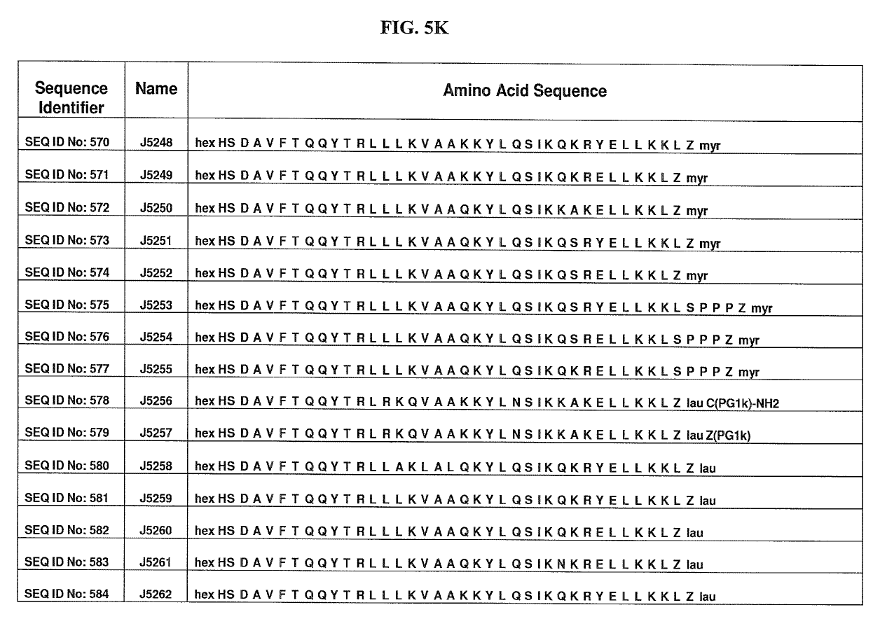

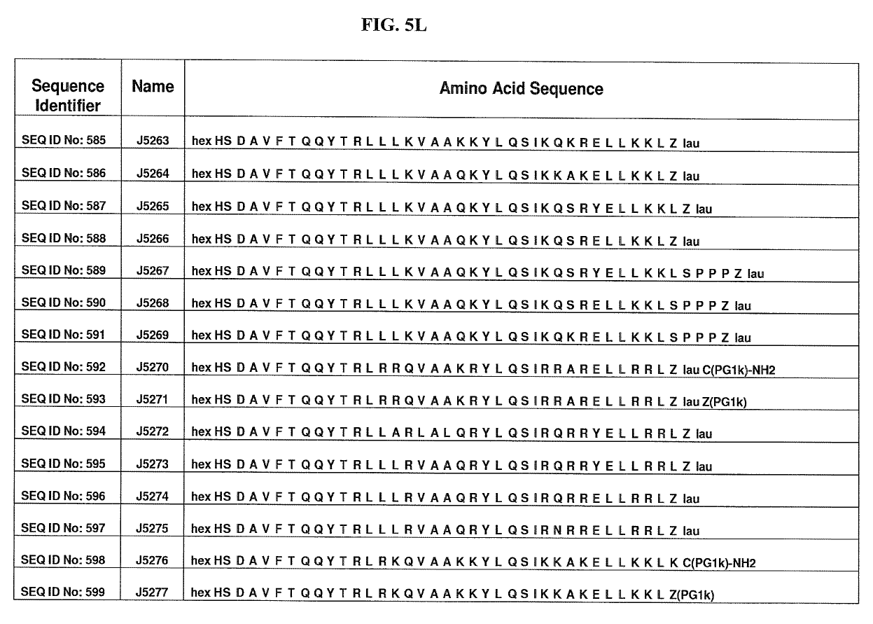

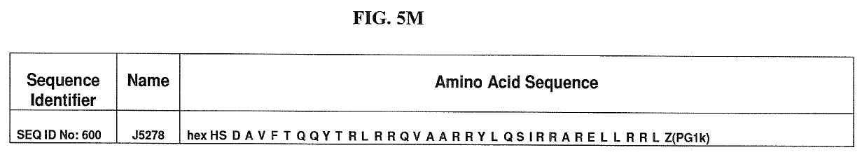

Finally, the ivDde group is removed from the C-terminal Lys by 2% hydrazine in DMF using a triple deprotection protocol (3 min/6 min/6 min; 75° max) and stearic acid is coupled using a triple coupling protocol. Final cleavage and deprotection is carried out using two rounds of microwave assisted cleavage with TFA/Water/TIS/3,6-dioxa-1,8-octanedithiol (92.5/2.5/2.5/2.5) for 18 ml at 38° C. each time. The crude product is precipitated and washed with cold diethylether, dissolved in distilled water and lyophilized to yield the product as a white powder. Yield: 140 mg crude yield of peptide product after lyophilization. Purification of the crude peptide is carried out by reverse-phase (C-18) HPLC using a gradient from 10 to 40% Solvent B (Solvent A: 0.1% TFA in water; Solvent B: 0.1% TFA in acetonitrile). Fractions are cut for purity from the major peak, pooled and lyophilized to yield the product as 25 mg of white powder (18% yield by weight from crude material). The purity is assessed by analytical reverse-phase HPLC as described above and is shown to be >95% (mass spec peak at M+1=4957/3 positive charge). FIG. 5 shows an HPLC trace of a crude peptide from a typical synthesis and pure peptide is typically obtained in 15 to 30% yield from crude peptide.

Other representative polypeptide analogs were prepared in a manner similar to that described above. Listed below in TABLE 2 are chemical properties of exemplary polypeptide analogs described herein. Such peptides are typically seen on mass spectroscopic readout as the M+4/4 and M+3/3 ions.

TABLE 2
Properties of Exemplary Polypeptide Analogs

| | Sequence | Molecular | | | |
|---|---|---|---|---|---|
| Name | Identifier | Weight | | Purity | Mass Spec |
| TP-135 | SEQ ID NO: 60 | 4955.1 | a.m.u. | 96.90% | |
| V2448 | SEQ ID NO: 95 | 5138 | a.m.u. | >99% | |
| A7275 | SEQ ID NO: 601 | 4473 | | >95% | |
| J5179 | SEQ ID NO: 555 | 4441 | | >95% | 1481.3 (m + 3), 1111.2 (m + 4), 889.3 (m + 5) |
| J5180 | SEQ ID NO: 556 | 4414 | | >95% | 1472.4 (m + 3), 1104.4 (m + 4), 883.8 (m + 5) |
| J5184 | SEQ ID NO: 558 | 4414 | | >95% | 1472.4 (m + 3), 1104.4 (m + 4), 883.8 (m + 5) |
| J5176 | SEQ ID NO: 554 | 4372 | | >95% | 1458.2 (m + 3), 1093.9 (m + 4), 875.4 (m + 5) |
| J5156 | SEQ ID NO: 552 | 4586 | | >95% | 1529.7 (m + 3), 1147.5 (m + 4), 918.3 (m + 5) |
| J5158 | SEQ ID NO: 553 | 4599 | | >95% | 1534.0 (m + 3), 1150.8 (m + 4), 920.8 (m + 5) |
| J5182 | SEQ ID NO: 557 | 4433 | | >95% | 1478.7 (m + 3), 1109.2 (m + 4), 887.7 (m + 5) |
| J5236 | SEQ ID NO: 560 | 4751 | | >95% | 1584.4 (m + 3), 1188.6 (m + 4), 951.0 (m + 5) |
| J5239 | SEQ ID NO: 561 | 4820 | | >95% | 1205.8 (m + 4), 964.9 (m + 5), 804.2 (m + 6) |
| V2493 | SEQ ID NO: 140 | 4740 | | >90% | 1581.5 (m + 3), 1186.4 (m + 4) |
| V2493 | SEQ ID NO: 140 | 4739 | | >90% | 1581.1 (m + 3), 1185.9 (m + 4), 949.0 (m + 5), |
| | | | | | 791.0 (m + 6), 678.0 (m + 7) |
| A7276 | SEQ ID NO: 602 | 4768 | | >90% | 1590.8 (m + 3), 1193.3 (m + 4), 955.0 (m + 5) |
| A7276 | SEQ ID NO: 602 | 4768 | | >90% | 1590.7 (m + 3), 1193.3 (m + 4), 954.7 (m + 5), |
| | | | | | 795.8 (m + 6) |
| A7277 | SEQ ID NO: 603 | 4796 | | >90% | 1600.1 (m + 3), 1200.3 (m + 4) |
| J5240 | SEQ ID NO: 562 | 4843 | | >90% | 1616.0 (m + 3), 1212.1 (m + 4) |
| J5240 | SEQ ID NO: 562 | 4843 | | >90% | 1615.4 (m + 3), 1211.8 (m + 4), 969.6 (m + 5), |
| | | | | | 808.3 (m + 6), 693.0 (m + 7) |
| J5241 | SEQ ID NO: 563 | 4576 | | >90% | 1526.6 (m + 3), 1145.0 (m + 4) |
| J5241 | SEQ ID NO: 563 | 4576 | | >90% | 1526.8 (m + 3), 1145.3 (m + 4), 916.3 (m + 5), |
| | | | | | 763.9 (m + 6) |
| A7278 | SEQ ID NO: 604 | 6084 | | >95% | 1521.4 (m + 4), 1217.8 (m + 5) |
| A7279 | SEQ ID NO: 605 | ~6844 | | >95% | envelop due to PEG heterogeneity |
| A7280 | SEQ ID NO: 425 | 5816 | | >95% | 1455.0 (m + 4), 1164.3 (m + 5) |
| A7281 | SEQ ID NO: 426 | ~6576 | | >95% | envelop due to PEG heterogeneity |
| L1400 | SEQ ID NO: 443 | 4691 | | >90% | 1564.9 (m + 3), 1174.1 (m + 4), 939.5 (m + 5), |
| | | | | | 783.6 (m + 6) |
| L1401 | SEQ ID NO: 444 | 4677 | | >90% | 1560.3 (m + 3), 1170.5 (m + 4), 936.6 (m + 5), |
| | | | | | 780.7 (m + 6), 669.3 (m + 7) |
| L1402 | SEQ ID NO: 445 | 4513 | | >90% | 1505.8 (m + 3), 1129.5 (m + 4), 903.9 (m + 5), |
| | | | | | 753.3 (m + 6) |
| L1403 | SEQ ID NO: 446 | 4499 | | >90% | 1501.3 (m + 3), 1126.2 (m + 4), 901.2 (m + 5), |
| L1403 | SEQ ID NO: 446 | 4499 | | >90% | 1501.3 (m + 3), 1126.2 (m + 4), 901.2 (m + 5), |
| | | | | | 751.2 (m + 6) |
| L1404 | SEQ ID NO: 447 | 4677 | | >90% | 1560.2 (m + 3), 1170.4 (m + 4), 936.6 (m + 5), |
| L1404 | SEQ ID NO: 447 | 4677 | | >90% | 1560.3 (m + 3), 1170.4 (m + 4), 936.6 (m + 5), |
| | | | | | 780.7 (m + 6) |
| L1405 | SEQ ID NO: 448 | 4514 | | >90% | 1505.8 (m + 3), 1129.7 (m + 4), 903.9 (m + 5), |
| | | | | | 753.3 (m + 6), 646.0 (m + 7) |
| L1405 | SEQ ID NO: 448 | 4514 | | >90% | 1506.1 (m + 3), 1129.7 (m + 4), 904.0 (m + 5), |
| | | | | | 753.5 (m + 6) |
| L1406 | SEQ ID NO: 449 | 4428 | | >90% | 1477.5 (m + 3), 1108.3 (m + 4), 887.0 (m + 5), |
| | | | | | 739.3 (m + 6), 633.8 (m + 7) |
| L1406 | SEQ ID NO: 449 | 4428 | | >90% | 1477.6 (m + 3), 1108.5 (m + 4), 887.0 (m + 5), |
| | | | | | 739.4 (m + 6) |
| L1407 | SEQ ID NO: 450 | 4636 | | >90% | 1546.5 (m + 3), 1160.1 (m + 4), 928.3 (m + 5), |
| | | | | | 773.8 (m + 6) |
| L1408 | SEQ ID NO: 451 | 4472 | | >90% | 1492.2 (m + 3), 1119.3 (m + 4), 895.8 (m + 5), |
| | | | | | 746.5 (m + 6), 640.0 (m + 7) |
| L1408 | SEQ ID NO: 451 | 4472 | | >90% | 1492.2 (m + 3), 1119.5 (m + 4), 895.9 (m + 5), |
| | | | | | 746.7 (m + 6) |
| L1409 | SEQ ID NO: 452 | 5014 | | >90% | 1672.8 (m + 3), 1255.0 (m + 4), 1004.0 (m + 5) |
| L1410 | SEQ ID NO: 453 | 4851 | | >90% | 1618.3 (m + 3), 1213.9 (m + 4), 971.4 (m + 5), |
| | | | | | 809.7 (m + 6), 694.2 (m + 7) |
| L1411 | SEQ ID NO: 454 | 4892 | | >90% | 1632.2 (m + 3), 1224.2 (m + 4), 979.8 (m + 5), |
| | | | | | 816.5 (m + 6), 699.9 (m + 7) |

The peptides of the invention are prepared in a similar manner.

### Example 6

### Recombinant Synthesis of the Polypeptides

Alternatively, the polypeptides described herein may be prepared by cloning and expression of a gene encoding for the desired polypeptide. In this process, a plasmid containing the desired DNA sequence is prepared and inserted into an appropriate host microorganism, typically a bacterium, such as E. coli, or a yeast, such as Saccharomyces cerevisiae, inducing the host microorganism to produce multiple copies of the plasmid, and so of the cDNA encoding for the polypeptide analogs described herein.

First, a synthetic gene coding for the selected PACAP or VIP analog is designed with convenient restriction enzyme cleavage sites to facilitate subsequent alterations. Polymerase chain reaction (PCR), as taught by Mullis in U.S. Pat. Nos. 4,683,195 and 4,683,202, incorporated herein by reference, may be used to amplify the sequence.

The amplified synthetic gene may be isolated and ligated to a suitable plasmid, such as a Trp LE plasmid, into which four copies of the gene may be inserted in tandem. Preparation of Trp LE plasmids is described in U.S. Pat. No. 4,738,921 and European Patent Publication No. 0212532, incorporated herein by reference. Trp LE plasmids generally produce 8-10 times more protein than Trp E plasmids. The multi-copy gene may then be expressed in an appropriate host, such as E. coli or S. cerevisiae.

Trp LE 18 Prot (Ile3, Pro5) may be used as an expression vector in the methods described herein. Trp LE 18 Prot (Ile3, Pro5) contains the following elements: a pBR322 fragment (EcoRI-BamHI) containing the ampicillin resistant gene and the plasmid origin of replication; an EcoRI-SacII fragment containing the trp promoter and the trpE gene; an HIV protease (Ile3, Pro5) gene fragment (SacII-HindIII); a bGRF gene fragment (HindIII-BamHI); and a transcription terminator from E. coli rpoc gene. The HIV protease and bGRF gene fragments are not critical and may be replaced with other coding sequences, if desired.

The expressed multimeric fusion proteins then accumulate intracellularly into stable inclusion bodies and may be separated by centrifugation from the rest of the cellular protein. VIP and PACAP related peptides do not denature so purification is straightforward through a combined ion exchange concentration/purification protocol followed by “polishing” on preparative reversed-phase high performance chromatography using a aqueous to aqueous-organic buffer gradient using 0.1% trifluoroacetic acid or 0.4M NH₄OAc (pH 4) as the pH modifier. The organic modifier used may be any of a number of water miscible solvents, for example acetonitrile, n-propanol, isopropanol, and the like, preferably n-propanol. The isolated fusion protein is converted to the monomeric PACAP or VIP analog by acylation with activated fatty acids and may be purified by cation exchange and/or reverse phase HPLC. The precise protocol is dependent on the particular sequence being synthesized. Typically the free amino terminus is less reactive than a Lys side chain, so differential acylation is straightforward. Alternatively, a fragment of the final sequence may be prepared in this way with subsequent condensation with a synthetically produced fragment containing the N- or C-terminal modifications. Chemical or “native” conjugations may be used (Dawson, P. E.; Muir, T. W.; Clark-Lewis, I.; Kent, S. B. Science 1994, 266, (5186), 776-9; Nilsson, B. L.; Soellner, M. B.; Raines, R. T. Annu Rev Biophys Biomol Struct 2005, 34, 91-118.).

Alternative methods of cloning, amplification, expression, and purification will be apparent to the skilled artisan. Representative methods are disclosed in Maniatis, et al., Molecular Cloning, a Laboratory Manual, 3rd Ed., Cold Spring Harbor Laboratory (2001), incorporated herein by reference.

### Example 7

### In Vitro Bioassay with Islet Cell Static Cultures

The following exemplary in vitro bioassay was conducted to evaluate the ability of representative polypeptide analogs to modulate insulin secretion.

Islet isolation. Rat islets were harvested (Sweet I R, et al. (2004) Biochem. Biophys. Res. Commun. 314, 976-983) from male Fisher rats weighing about 250 g and which were anesthetized by intraperitoneal injection of sodium pentobarbital (35 mg/230 g rat). Generally, the islets were prepared by injecting collagenase (10 mL of 0.23 mg/mL Liberase, Roche Molecular Biochemicals, Indianapolis, Ind.) into the pancreatic duct of the partially dissected pancreas and surgically removing it. All procedures were approved by the Institutional Animal Care and Use Committee at the University of Washington.

The pancreata were placed into 15 mL conical tubes containing 5 mL of 0.23 mg/mL Liberase and incubated at 37° C. for 30 min. The digestate was then filtered through a 400-micrometer stainless steel screen, rinsed with Hanks' buffered salt solution, and purified in a gradient solution of Optiprep™ (Nycomed, Oslo, Norway). Islets were cultured for 18-24 h prior to performing the assay in RPMI Media 1640 supplemented with 10% (v/v) heat inactivated fetal bovine serum (FBS), antibiotic-antimycotic (100 U/mL penicillin, 1001 g/mL streptomycin, and 0.25 lg/mL amphotericin B), 2 mM glutamine (all from Gibco-BRL, Grand Island, N.Y.), and 1 mM p-mercaptoethanol.

Bioassay. Islets were picked under a microscope and placed into 10 ml 3 mM Krebs Ringer Buffer (KRB) solution for washing. Islets were incubated in 3 mM glucose KRB for 60 min and then groups of 10 islets per well were placed into 200 μl media in a 96-well plate. The islets were incubated for 120 min under control or treatment conditions, and supernatants were collected. A typical set of conditions tested 3 mM glucose (resting control), 16 mM glucose (testing control), 16 mM glucose+10 nm GLP1, 16 mM glucose+10 nM Exendin-4, 16 mM glucose+50 nM test peptide. The buffer conditions were KRB with 0.1% BSA, 20 mM HEPES and the assay is performed in quadruplicate. Supernatants were evaluated for insulin content using a commercial insulin enzyme-linked immunosorbent (ELISA) assay per manufacturer's directions.

Results of Bioassay. TABLE 3 illustrates the insulin secretion obtained in the above assay for analog TP-106, which exhibited maximal activity in this assay at a concentration of 200 nM. For comparison, Exendin 4 was tested in this assay and showed maximal activity at 10 nM. TP-106 is a highly hydrophobic analog, designed to depot in the site of sc injection and therefore the effective concentration of TP-106 is expected to be much lower than the nominal concentration (200 nM).

TABLE 3
Results of Islet Cell Static Culture Bioassay with TP-106

| | Insulin secreted | Standard |
|---|---|---|
| | (ng/100 islets/min) | Deviation |
| 3 mM glucose | 0.01 | 0.00 |
| 16 mM glucose | 1.38 | 0.17 |
| Exendin 4 + 16 mM glucose | 4.82 | 0.20 |
| 50 nM TP-106 + 16 mM glucose | 2.72 | 0.60 |
| 200 nM TP-106 + 16 mM glucose | 5.20 | 0.50 |
| 16 mM glucose + 16 mM glucose | 1.58 | 0.05 |

The islet cell static culture assay described above is performed on additional exemplary polypeptide analogs. TP-107 exhibited maximal activity in this assay at a concentration of 100 nM. For comparison, Exendin 4 is tested in this assay and showed maximal activity at 10 nM. Presented peptides are designed to bind to serum albumin and thus, the concentration of free peptide to impart insulin activity is expected to be much lower and therefore the analog more potent than indicated in this in vitro assay. Similar observations have been reported during studies with the hydrophobic peptide, insulin detimir (Kurtzhals, P., et al., Diabetes 49:999-1005 (2000)).

TABLE 4
Results of Islet Cell Static Culture Bioassay with TP-107 and TP-108

| | Average | |
|---|---|---|
| | Insulin secreted | Standard |
| | (ng/100 islets/min) | Deviation |
| 3 mM glucose | 0.14 | 0.00 |
| 16 mM glucose | 3.65 | 0.80 |
| 10 nM Exendin 4 + 16 mM glucose | 6.75 | 1.15 |
| 10 nM PACAP + 16 mM glucose | 6.07 | 1.67 |
| 10 nM TP-107 + 16 mM glucose | 2.89 | 0.21 |
| 100 nM TP-107 + 16 mM glucose | 6.10 | 1.55 |
| 1 uM TP-107 + 16 mM glucose | 6.07 | 0.90 |
| 100 nM TP-108 + 16 mM glucose | 4.10 | 1.21 |
| 1 uM TP-108 + 16 mM glucose | 5.65 | 0.13 |

### Example 8

### In Vitro Flow Assay

Static assays may suffer from feedback loop suppression of secretion of insulin or other hormones. Therefore in vitro flow assay conditions are useful in order to confirm the results of static assays. Thus islets are isolated as described in Example 7 and seeded into a flow apparatus as described (Sweet, I., et al., Diabetes 53: 401-9 (2004)). The islet flow culture system (Sweet, I., et al., Diabetes Technol Ther. 4: 67-76 (2002)) includes a pump, gas equilibrator, a glass islet perifusion chamber, detectors for oxygen and cytochromes, and a fraction collector. Islets are stabilized with Cytopore beads (Amersham Biosciences, Piscataway, N.J.) that are layered into the chamber using a P200 pipette as follows: First, 0.4 mg of beads in 20 μl media are allowed to settle onto the porous polyethylene frit at the chamber's bottom. A mixture of 600 islets and Cytodex beads (0.12 mg; Amersham Biosciences) is added followed by another 0.4 mg Cytopore beads and a top frit. Porous frits are cored (0.3 cm) from polyethylene sheets (Small Parts, Miami Lakes, Fla.). Typically 600 or 300 islets are used but the number can be varied depending on the compounds being assayed and the number of supernatant samples desired. Krebs Ringer or RPMI media at a flow rate of 200 μL per min. The islets are challenged with 16 mM glucose solution and then with test compound in 16 mM glucose containing buffer. Samples are taken from the effluent from the chamber and assayed for insulin content using an enzyme-linked immunosorbent assay according to the manufacturer's instructions (ALPCO, Windham, N.H.). Table 5 illustrates the substantial glucose-dependent insulin secretion stimulated by test peptides that are within the scope of and representative of the invention, i.e., TP-128 and V2449.

TABLE 5
Results of Islet Flow Culture Bioassay with TP-128 and V2449.

| | Insulin secreted (ng/100 islets/min) |
|---|---|
| 3 mM glucose | 0.5 |
| 16 mM glucose | 1 |
| 100 nM TP-128 + 16 mM glucose | 14 |
| 100 nM V2449 + 16 mM glucose | 12 |

### Example 9

### In Vivo Bioassay

The following exemplary in vivo assay was conducted to evaluate the ability of representative polypeptide analogs to modulate insulin secretion.

Tested Study Groups. Naive, 8 weeks old female db/db mice were acclimated for one week, during which period animals were handled periodically to allow them to be acclimated to experiment procedures. Study groups contained 6 mice per group and were administered with one of the following by intraperitoneal injection:

(1) Vehicle control;

(2) Positive control (exendin-4 or other standard treatment);

(3) Polypeptide Analog at high dose; or

(4) Polypeptide Analog at low dose.

A small volume of blood was taken from a cut at the tip of tail for blood sampling. Blood glucose levels were determined on a commercial, hand-held glucose meter. On Day 1, animals were injected with polypeptide analogs and controls in the morning. Blood samples were taken and analyzed immediately before injection and at 2, 4, 8, 14, and 24 hours after injection. Animals were allowed to feed, ad libitem, throughout the assay (Tsutsumi et al., Diabetes 51:1453-60 (2002)).

TABLE 6 lists a representative sampling of the data obtained from the in vivo assay described above. As shown below, TP-106 exhibited statistically significant activity (e.g., reduced plasma glucose) at a high dose 2 hr after injection and maintains activity at 4 hrs post dosing.

TABLE 6
Results of In Vivo Assay with TP-103 and TP-106
Mean Blood Glucose Levels (mmol/L)

| | 0 hr | 2 hr | 4 hr | 8 hr | 14 hr | 24 hr |
|---|---|---|---|---|---|---|
| Vehicle | 23.9 | 21.9 | 18.3 | 27.3 | 22.5 | 23.5 |
| | s.d.* = 1.33 | s.d. = 1.22 | s.d. = 1.01 | s.d. = 1.52 | s.d. = 1.25 | s.d. = 1.31 |
| TP-103 Low dose | 22.9 | 20.5 | 17.6 | 26.4 | 24.6 | 21.4 |
| | s.d. = 1.27 | s.d. = 1.14 | s.d. = 0.98 | s.d. = 1.47 | s.d. = 1.37 | s.d. = 1.19 |
| TP-103 High dose | 20.7 | 17.3 | 16.9 | 23.4 | 23.7 | 25.0 |
| | s.d. = 1.15 | s.d. = 0.96 | s.d. = 0.94 | s.d. = 1.30 | s.d. = 1.31 | s.d. = 1.39 |
| TP-106 Low dose | 23.9 | 20.5 | 16.1 | 24.0 | 28.2 | 23.2 |
| | s.d. = 1.33 | s.d. = 1.14 | s.d. = 0.89 | s.d. = 1.33 | s.d. = 1.57 | s.d. = 1.29 |
| TP-106 High dose | 21.8 | 13.4 | 14.7 | 25.1 | 26.3 | 21.2 |
| | s.d. = 1.21 | s.d. = 0.75 | s.d. = 0.82 | s.d. = 1.39 | s.d. = 1.46 | s.d. = 1.18 |

*s.d. = standard deviation

### Example 10

### Relaxation of Guinea Pig Tracheal Smooth Muscle

Tracheal tissue is removed from Hartley guinea pigs (500-700 g) after sacrificing them with an overdose of urethane (O'Donnell, M., et al. J. Pharmacol. Exptl. Therapeut. 270:1282-8 (1994)). The trachea is divided into four ring segments. Each ring is suspended by stainless steel wires in a 10 mL jacketed tissue bath and attached to a Grass force displacement transducer for isometric recording of tension. The smooth muscle tissue is bathed in modified Kreb's-Hanseleit solution at 37.5° C. with constant bubbling of O₂/CO₂ (95:5). Tracheal rings are placed under a resting tension of 1.5 g and readjusted as required. Tissues are precontracted with carbachol (30 nM) or KCl (10 mM) and treated with the test agent. The difference intension between the precontraction induced by carbachol and the level during a final maximum theophyline-induced relaxation (1 mM) is regarded as 100% active tension.

Paired concentration response experiments are carried out for the test peptide and standard VIP. The concentration of the test peptide and the VIP strandard are increased cumulatively as soon as the peak drug response is observed. Relaxant responses are expressed as a percentage of relaxation relative to the 100% active tension and EC50 values are determined by linear regression.

### Example 11

### Selective PEGylation of a VPAC2 Agonist to Prepare P307

(SEQ ID NO: 315)

Hexanoyl-His-Ser-Asp-Ala-Val-Phe-Thr-Asp-Gln-Tyr-
Thr-Arg-Leu-Leu-Lys-Gln-Val-Ala-Ala-Lys-Lys-Tyr-
Leu-Asn-Ser-Ile-Lys-Lys-Ala-Lys-Arg-Leu-Leu-Arg-
Lys-Leu-Lys(stearoyl)-Cys(PEG1K)-NH₂

The cysteine containing precursor to P307 is prepared in the free SH form according to the microwave-assisted synthesis procedure of Example 5. A sample of 55 mg of P307 precursor is dissolved in 100 mL of 100 mM phosphate buffer at pH 7.5 (containing 15 mM disodium ethylenediaminetetraacetic acid) that is deaerated by argon bubbling, and treated with 70 mg of PEG1150 (MeO-PEG-maleinimide; PEG-WM 750 Da; IRIS Biotech) during a period of approximately 3 hr. The reaction is monitored by Ellman reagent to detect disappearance of SH functional groups and purified by size exclusion chromatography on a 300 mL column of Sephadex 2000 swollen with phosphate buffer. The effluent is followed by uv absorption and cut for purity (early peaks) to remove unreacted PEG and smaller molecular weight impurities. Further purification by ion exchange chromatography (for example carboxymethylcellulose, CM Sepharose, or the like) or preparative HPLC is available is preferred. The solution of product in elution buffer is dialyzed (lkDa cut-off membrane; Amersham) against a suitable buffer (e.g. acetate, pH5) and lyophilized to yield the product as a white powder. The protein conjugate is characterized by analysis on a PolyCAT A column (Nest Group).

### Example 12

### Selective PEGylation of a VPAC2 Agonist to Prepare P4819

(SEQ ID NO: 253)

Hexanoyl-His-Ser-Asp-Ala-Val-Phe-Thr-Gln-Gln-Tyr-
Thr-Arg-Leu-Arg-Lys-Gln-Val-Ala-Ala-Lys-Lys-Tyr-
Leu-Asn-Ser-Ile-Lys-Lys-Ala-Lys-Glu-Leu-Leu-Lys-
Lys-Leu-Lys(ε-stearoyl)-Cys(PEG1k)-NH₂

A similar protocol for PEGylation at a cysteine residue is based on that of Tom, I, et al. (2007) AAPS Journal, 9: E227-34. Briefly, 8.3 mg of the peptide corresponding to that in the title, but with an unmodified cysteine residue at the C-terminus (SEQ ID 562 in 2 mL of 10 mM sodium phosphate, pH 6 and added to a solution of an 8 fold molar excess (17.07 mg) of 1239 Da m-dPEGtm24-MAL (Quanta BioDesign, Powell, Ohio), in 1.428 mL of the same buffer. The final volume was 3.428 mL or 500 μM in the peptide and 4 mM in PEG1239. The course of the PEGylation was monitored by use of the Ellman reagent and was allowed to go to near completion before being terminated by the addition of excess cysteine (185 μL of 150 mM cysteine). The PEGylated peptide was purified by cation exchange chromatography. Thus 1.5 ml of CM Sepharose Fast Flow resin (GE healthcare) was equilibrated with 10 column volumes of 20 mM sodium acetate, pH 6. The reaction mixture was incubated with the resin for up to 30 minutes after being loaded onto the column. The column was washed with 10 column volumes of 20 mM sodium acetate, pH 6. The PEGylated peptide product was eluted with a step gradient of 2 column volumes of 0.35 M and 0.4 M, and then 3 column volumes of 0.45 M and 0.5 M sodium chloride in 20 mM sodium acetate, pH 6. Fractions of 750 μL were collected, and run on NUPAGE Novex 12% Bis-tris gel (Invitrogen). Fractions with a single band corresponding to a molecular weight of 6 kD were pooled, desalted by dialysis against water using an Amicon Ultra-15 Centrifugal Filter Unit with Ultracel-3 membrane (Millipore), and lyophilized. The purity of PEGylated peptide was verified by RP-HPLC and mass spectrometry. The desired product was obtained as a fluffy white powder (4.4 mg, 53%) showing greater than 95% purity by RP-HPLC and with the expected protonated molecular ion of 1217.8 and 1521.4 for the product as M/+5 and M/+4 forms with the defined molecular weight PEG added (MW=6084).

In a similar manner, but incorporating 8.86 mg of the starting, cysteine-containing peptide (SEQ ID 562 and 37.82 mg of 2 kDa MeO-PEG-mal (IRIS Biotech Gmbh, Germany) and following a similar purification was obtained 6.42 mg (72% yield) of the corresponding PEG 2000 modified peptide as a white lyophilized powder. Characterization of the PEG2000 modified peptides is less clear-cut since the PEG is not a defined molecular weight, single molecular class, rather a mixture. None-the less there is a clear movement to a higher average molecular weight by gel, a broad peak on the hplc column and a higher average molecular mass seen by mass spectroscopy. This product is another variant of P4819 (SEQ ID: 253), but with slightly longer and more heterogeneous PEG modification.

In a similar manner, but using 11.85 mg of the C-terminal cysteine-containing precursor (SEQ ID 563) of peptide SEQ ID:425 and 24.78 mg of 1239 Da m-dPEGtm24-MAL and following similar purification was obtained 4.23 mg (36% yield) of the desired modified peptide (SEQ ID:425) as a lyophilized white powder showing greater than 95% purity by RP-HPLC and with the expected molecular ion of 1164.3 and 1455.0 for the product as M/+5 and M/+4 forms with the defined molecular weight PEG added (MW=5820).

In a similar manner, but incorporating 9.36 mg of the starting, cysteine-containing precursor of peptide (SEQ No: 563) and 41.5 mg of 2 kDa MeO-PEG-mal (IRIS Biotech Gmbh, Germany) and following a similar purification was obtained 4.8 mg (51% yield) of the corresponding PEG 2000 modified peptide (SEQ ID: 426) as a white lyophilized powder. Characterization of the PEG2000 modified peptides is less clear-cut since the PEG is not a defined molecular weight, single molecular class, rather a mixture. None-the less there is a clear movement to a higher average molecular weight by gel, a broad peak on the HPLC column and a higher average molecular mass seen by mass spectroscopy.

TABLE 7
Properties of Exemplary PEGylated Polypeptide Analogs

| | Sequence | Molecular | | |
|---|---|---|---|---|
| Name | Identifier. | Weight | Purity | Mass Spec |
| P4819 | SEQ ID NO: 253 | 6084 | >95% | 1521.4 (m + 4), |
| | | | | 1217.8 (m + 5) |
| A7279 | SEQ ID NO: 605 | ~6844 | >95% | envelope due to |
| | | | | PEG heterogeneity |
| A7280 | SEQ ID NO: 425 | 5816 | >95% | 1455.0 (m + 4), |
| | | | | 1164.3 (m + 5) |
| A7281 | SEQ ID NO: 426 | ~6576 | >95% | envelope due to |
| | | | | PEG heterogeneity |

In a similar manner are prepared the cysteine-PEGylated compounds of the invention.

### Example 13

### Pharmacokinetic Evaluation of Test Peptides

Test peptides were labeled with ¹²⁵I at a commercial house using standard protocols (PerkinElmer Life Sciences) that employ ¹²⁵I and an oxidizing agent such as chloramine T. Such protocols (Hunter, W M and Greenwood, F C, Nature 194: 495 (1962) are standard in the literature/industry and are best carried out at a specialized radiolabeling house. The products were purified by open column size-exclusion to remove iodine and reversed-phase hplc under standard gradient conditions of organic modified buffer, for example CH₃CN in 0.1% CF₃CN, with the gradient dependent on the particular peptide. The pure radiolabeled peptides were typically found to be in the range of 2200 Ci/mmol and were lyophilized from a buffer solution, for example 50 mM sodium phosphate at pH7.4 containing 0.2M NaCl, 1 M glycine, 0.25% BSA, and 500 KIU/mL aprotinin prior to shipment.

For dose formulation on the day of dosing, an appropriate volume of the stock radioactive test peptide (circa 1000 microCi/mL in distilled H₂O) was added to an appropriate volume of stock of the unlabeled test article in a silanized tube and mixed gently by inversion to obtain a homogeneous solution. The resulting dose formulation contained 10 mL of solution of circa 8.89 nmol/mL of test article at a specific activity of 9 Ci/mmol and a radioactivity concentration of circa 80 microCi/mL. This formulation can be scaled up or down. Two predose and two post-dosing samples of 0.100 mL were taken for radioactive analysis.

The animals chosen for the study were a standard rat strain (for example Charles River CD(SD)) and were acclimated to the lab environment prior to use. The rats were anesthetized with isoflurane vapors to effect using a precision vaporizer (3-5%, for approximately 5-10 min) and suspended by the upper incisors on an incline rack in a supine position. The rat's tongue was retracted to the side to allow access to the back of the throat and the throat was illuminated with an appropriate lighting device. A 20 G×32 mm Abbocath-T (or equivalent) cannula (sheath only) attached to a glass tuberculin syringe was inserted into the trachea. Placement in the airway was conferment by “pulling/pushing” the barrel of the glass syringe. Nor resistance of the barrel assures placement in the airway. Resistance of the barrel indicates placement in the esophagus; the cannula must be removed and the procedure reinitiated. Once the cannula is confirmed in the airway, the class syringe is removed. A syringe (containing the appropriate volume of the test article) with a 22 G×1 in. blunt needle attached, was inserted into the hub end of the cannula and the test peptide is delivered into the trachea by pushing the plunger of the syringe. The needle and the syringe are retracted from the hub of the cannula and the test article is displaced further into the airway by “injection” one mL of air with a clean syringe (no needle). The rat may remain suspended on the incline rack for an additional 10-20 sec to allow further distribution of the test peptide into the lungs. The rat was then returned to its home cage.

At the scheduled sacrifice times (0.25, 1, 4, 10, 24 hrs post dose), whole blood was collected (less than 10 mL) from anesthetized rats (n=3 rats/group/time-point) via cardiac puncture and stored using K3-EDTA as the anticoagulant. Rats were then euthanized by cervical dislocation. The stomach (with contents) and lungs/trachea/bronchi of each rat were collected for radioanalysis. All samples were stored at or below −10° C. One half of the blood was reserved for analysis of radioactivity and the other half was centrifuged to obtain plasma. Aliquots of dose formulation, whole blood, plasma, and tissue were placed directly in tubes for gamma scintillation counting and analyzed directly for radioactivity.

An example of typical data for the peptides of the invention from this assay was obtained as a graph of the pharmacokinetic behavior of exemplary peptides exhibiting their long duration of action (figure not shown; FIG. 8 of International publication WO2008/043102). It is known from literature studies (Refai, E., et al. Nucl Med Biol 26: 931-6 (1999)) that VIP, homologous to the peptides of the invention, has a t_{1/2} on the order of 0.6 min when administered i.v. In the above-noted graph, the peptides of the invention exhibited a very prolonged duration of action with t_{1/2} values on the order of hours, rather than the 0.6 to several minutes typical for VIP or PACAP. Such a prolonged duration of action is important for the use of these described peptides in the treatment of animal and human disease.

### Example 14

### Uses of the Invention

The polypeptides described herein are useful for the prevention and treatment of a variety of diseases and disorders. These include metabolic disorders, asthma, COPD and primary hypertension. In particular, the compounds described herein are indicated for the prophylaxis and therapeutic treatment of: elevated blood glucose levels, hyperglycemia, dyslipidemia, hypertriglyceridemia, diabetes, including Type 2 Diabetes Mellitus, Metabolic Syndrome (Grundy, S. M., et al. Nature Rev. Drug Disc. 5: 295-309 (2006)), Maturity Onset Diabetes of the Young (MODY, Herman, W. H., et al, Diabetes 43:40-6 (1994); Fajans, S. S., et al. Diabet Med. 13 (9 suppl 6): s90-5 (1996)), Latent Autoimmune Diabetes Adult (LADA; Zimmet, P. Z., et al., Diabetes Med. 11:299-303 (1994); impaired glucose tolerance (IGT); impaired fasting glucose (IFG); gestational diabetes (Rumbold, A. R. and Crowther, C. A., Aust N. Z. J. Obstet. Gynaecol. 41: 86-90)); Syndrome X, insulin resistance, stimulate proliferation of beta cells, improve beta cell function, activate dormant beta cells, metabolic acidosis and obesity. The polypeptides described herein are useful for prevention and treatment of secondary causes of diabetes and other metabolic diseases such as glucocorticoid excess, growth hormone excess, pheochromocytoma and drug-induced diabetes (for example due to pyriminil, nicotinic acid, glucocorticoids, phenyloin, thyroid hormone, β-adrenergic agents, α-interferon and drugs used to treat HIV infection).

The polypeptides of the present invention are also useful for treating complications caused by diabetes and/or the metabolic syndrome such as atherosclerotic disease, hyperlipidemia, hypercholesteremia, low HDL levels, hypertension, cardiovascular disease (including atherosclerosis, coronary heart disease, coronary artery disease, and hypertension), cerebrovascular disease and peripheral vessel disease; and for the treatment of lupus, polycystic ovary syndrome, carcinogenesis, and hyperplasia, asthma, male and female reproduction problems, sexual disorders, ulcers, sleep disorders, disorders of lipid and carbohydrate metabolism, circadian dysfunction, growth disorders, disorders of energy homeostasis, immune diseases including autoimmune diseases (e.g., systemic lupus erythematosus), as well as acute and chronic inflammatory diseases, rheumatoid arthritis, and septic shock.

The polypeptides of the present invention are also useful for treating physiological disorders related to, for example, cell differentiation to produce lipid accumulating cells, regulation of insulin sensitivity and blood glucose levels, which are involved in, for example, abnormal pancreatic beta-cell function, insulin secreting tumors and/or autoimmune hypoglycemia due to autoantibodies to insulin, autoantibodies to the insulin receptor, or autoantibodies that are stimulatory to pancreatic beta-cells, macrophage differentiation which leads to the formation of atherosclerotic plaques, inflammatory response, carcinogenesis, hyperplasia, adipocyte gene expression, adipocyte differentiation, reduction in the pancreatic beta-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

The polypeptides of the present invention are useful for the prevention and treatment of a variety of inflammatory disorders, defined broadly. In particular the compounds of the present invention are indicated for the prophylaxis and therapeutic treatment of asthma (Linden A, et al. (2003). Thorax 58: 217-21), cardioprotection during ischemia (Kalfin, et al., J Pharmacol Exp Ther 1268: 952-8 (1994); Das, et al., Ann NY Acad Sci 865: 297-308 (1998)), primary pulmonary hypertension (Petkov, V., et al. J Clin Invest 111: 1339-46. (2003)), and the like.

As indicated above, the lung is an important new medical target for treatment by VPAC2 agonists. For example, asthma is a large and rapidly growing disease but the current methods of treatment carry substantial risk of serious side effects. Studies both in vitro and in vivo with animal models showed that VPAC2 selective agonists cause prompt relaxation of tracheal smooth muscle preconstricted with carbachol, histamine or KCl (O'Donnell, K., et al., J. Pharmacol. Exptl. Therapeut. 270: 1282-8 (1994) and Example 10) as well as in sensitized guinea pigs (O'Donnell, K., et al., J. Pharmacol. Exptl. Therapeut. 270: 1289-94 (1994)). Human bronchial tissue responds similarly to PACAP analogs (Yoshihara, S., et al., Regulatory Peptides 123: 161-5 (2004)). Treatment of asthma patients with a VPAC2 selective molecule showed prompt bronchodilatation and a similar maximal effect to that shown by a leading β2 adrenoceptor agonist, formoterol (Linden, A., et al. Thorax 58: 217-21 (2003)). While β2 adrenoceptor agonists are effective bronchodilators, they have black box warnings for sudden death. In contrast, no clinically significant side effects are seen for the VPAC2 agonist. However it is short acting and therefore could not be developed commercially. In contrast, the compounds described herein are designed to have high VPAC2 selectivity, long duration of action, and to be permeable into lung tissue thus making them attractive drug development candidates for treatment of asthma and other obstructive diseases of the lung.

Another important activity of VPAC2 agonists is their ability to suppress the proinflammatory response of mast cells in response to inflammatory signals like bacterial lipopolysaccharide (Delgado, M. and Ganea, D., J. Immunol. 167: 966-75 (2001)). Mast cells are thought to be important effectors in asthma (Kraft, M., et al., Chest 124: 42-50 (2003)) as well as in chronic obstructive pulmonary disease (COPD), based on recent research (Barnes, P. J., J. COPD 1: 59-70 (2004)). The compounds of the present invention are novel, disease modifying treatments for both of these important lung diseases, asthma and COPD as well as for the treatment of other respiratory conditions.

Pulmonary hypertension is an important disease caused by increased vascular resistance in the pulmonary arteries. This can be caused either by some common conditions—congenital heart defects, scleroderma, HIV infection, blood clots, liver disease, etc. (secondary pulmonary hypertension; SPH) or by unknown causes (primary pulmonary hypertension; PPH). While PPH is a rare disease, SPH is a major disease category with unmet medical needs (Benisty, J. I., Circulation 106: e192-4 (2002)). Research in PPH has demonstrated that VIP has an important beneficial effect on exercise time /distance (Petkov V, et al., J Clin Invest 111: 1339-46 (2003)). The long acting VPAC2 analogs of the present invention will have a similar beneficial effect in the treatment of such diseases and disease and this effect will be extended to SPH.

In another embodiment, the polypeptides described herein may be administered in combination with other compounds useful in the treatment of metabolic disorders. For example, the polypeptides described herein may be administered with one or more of the following compounds used in the treatment of metabolic disorders, including but not limited to insulin, insulin analogs, incretin, incretin analogs, glucagon-like peptide, glucagon-like peptide analogs, glucose dependent insulinotropic peptide analogs, exendin, exendin analogs, sulfonylureas, biguanides, α-glucosidase inhibitors, thiazolidinediones, peroxisome proliferator activated receptor (PPAR, of which includes agents acting on the α, β, or γ subtypes of PPAR receptors and/or those agent acting on multiple subtypes of the PPAR receptors) agonists, PPAR antagonists and PPAR partial agonists may be administered in combination with the polypeptides of the present invention. In order to clarify the types of pharmaceutical agents mentioned by the general terms above, specific examples are given. For example, Eli Lilly sells a fast-acting insulin analog called “lispro” under the trade name Humalog® and Novo Nordisk sells another fast-acting insulin analog called “aspart” under the trade name NovoLog®. In addition, Aventis sells a long-acting insulin analog called “glargine” under the trade name Lantus® and Novo Nordisk sells another long-acting insulin analog called “detemir” under the trade name Levemir®. Examples of incretin analogs (GLP1 or GIP analogs) are exendin-4 (BYETTA® Amylin Pharmaceuticals, Inc., San Diego, Calif.), liraglutide, ZP-10 (AVE-010), albugon, and the like. Examples of sulfonylureas and the insulin secretagogues known as glinides are Glipizide, Gliclazide, Glibenclamide (glyburide), Glimepiride, and the glinides Repaglinide, and Nateglinide). Examples of the “biguanides” are metformin (Glucophage), buformin, and phenformin. Examples of “α-glucosidase inhibitors” are acarbose (Precose) and miglitol (Glycet). Examples of currently marketed PPARγ pharmaceuticals are the thiazolidinediones pioglitizone (Actos) and rosiglitazone (Avandia).

The term “insulin” as used herein includes, but not limited to, insulin analogs, natural extracted human insulin, recombinantly produced human insulin, insulin extracted from bovine and/or porcine sources, recombinantly produced porcine and bovine insulin and mixtures of any of these insulin products, and likewise include all the specific examples disclosed in the previous paragraphs. The term is intended to encompass the polypeptide normally used in the treatment of diabetics in a substantially purified form but encompasses the use of the term in its commercially available pharmaceutical form, which includes additional excipients. The insulin is preferably recombinantly produced and may be dehydrated (completely dried) or in solution.

The terms “insulin analog,” “monomeric insulin” and the like are used interchangeably herein and are intended to encompass any form of “insulin” as defined above, wherein one or more of the amino acids within the polypeptide chain has been replaced with an alternative amino acid and/or wherein one or more of the amino acids has been deleted or wherein one or more additional amino acids has been added to the polypeptide chain or amino acid sequences, which act as insulin in decreasing blood glucose levels. In general, the term “insulin analogs” of the present invention include “insulin lispro analogs,” as disclosed in U.S. Pat. No. 5,547,929, incorporated hereinto by reference in its entirety; insulin analogs including LysPro insulin and humalog insulin, and other “super insulin analogs”, wherein the ability of the insulin analog to affect serum glucose levels is substantially enhanced as compared with conventional insulin as well as hepatoselective insulin analogs which are more active in the liver than in adipose tissue. Preferred analogs are monomeric insulin analogs, which are insulin-like compounds used for the same general purpose as insulin, such as insulin lispro, i.e., compounds which are administered to reduce blood glucose levels.

“Insulin analogs” are well known compounds. Insulin analogs are known to be divided into two categories: animal insulin analogs and modified insulin analogs (pages 716-20, chapter 41, Nolte M. S. and Karam, J. H., “Pancreatic Hormones & Antidiabetic Drugs” In Basic & Clinical Pharmacology, Katzung, B. G., Ed., Lange Medical Books, New York, 2001). Historically, animal insulin analogs include porcine insulin (having one amino acid different from human insulin) and bovine insulin (having three amino acids different from human insulin) which have been widely used for treatment of diabetes. Since the development of genetic engineering technology, modifications are made to create modified insulin analogs, including fast-acting insulin analogs or longer acting insulin analogs.

Several insulin analog molecules have been on the market prior to the filing date of the subject application. For example, Eli Lilly sells a fast-acting insulin analog called “lispro” under the trade name Humalog® and Novo Nordisk sells another fast-acting insulin analog called “aspart” under the trade name NovoLog®. In addition, Aventis sells a long-acting insulin analog called “glargine” under the trade name Lantus® and Novo Nordisk sells another long-acting insulin analog called “detemir” under the trade name Levemir®. Table 41-4 of the article by Nolte and Karam (2001) referenced above illustrates the wide range of types of molecules generically referred to as insulin preparations.

The term “incretin analogs” refers to incretin hormones responsible for the phenomenon of enhanced insulin secretion in the presence of food in the gut and this action (GLP-1 and GIP) is widely known (e.g. articles referenced in Creutzfeldt, W, “The [pre-]history of the incretin concept”. Regulatory Peptides 128: 87-91 (2005). Examples of incretin analogs (GLP1 or GIP analogs) are exendin-4 (BYETTA® Amylin Pharmaceuticals, Inc., San Diego, Calif.), liraglutide, ZP-10 (AVE-010), albugon, and the like.

The term “glucagon-like peptide analogs” refers to well known analogs of Glucagon-Like Peptide (GLP1) (e.g. Nourparvar, A., et al. “Novel strategies for the pharmacological management of type 2 diabetes” Trends in Pharmacological Sciences 25, 86-91 (2004)), and reviews of the area discussed their range of structure and function in detail (cf Table 1 in Knudsen, L. B. “Glucagon-like Peptide-1. The Basis of a New Class of Treatment for Type 2 Diabetes”. J. Med. Chem. 47: 4128-4134 (2004) and references therein). Examples of “glucagon-like peptide analogs” include Liraglutide, Albugon, and B™-51077.

The term “exendin analogs” refers to exendin (also known as exendin-4, exanetide, (BYETTA® (Amylin Pharmaceuticals, Inc., San Diego, Calif.) and its analogs which have been major diabetes research objectives (c.f. Thorkildsen C. “Glucagon-Like Peptide 1 Receptor Agonist ZP10A Increases Insulin mRNA Expression and Prevents Diabetic Progression in db/db Mice”. J. Pharmacol. Exptl. Therapeut. 307: 490-6 (2003)). Exendin is known to be a specific type of glucagon-like peptide-1 mimic. For example, ZP-10 (AVE-010) is an exendin analog that binds to the GLP1 receptor.

The term “sulfonylureas” refers to well known sulfonylureas used for many years in the treatment of type 2 diabetes. Extensive clinical trial literature and reviews of sulfonylureas are available (c.f. Buse, J., et al. “The effects of oral anti-hyperglycaemic medications on serum lipid profiles in patients with type 2 diabetes”. Diabetes Obesity Metabol. 6: 133-156 (2004)). In table 1 in the Buse reference, the major sulfonylureas/glinides are listed chronologically as Glipizide, Gliclazide, Glibenclamide (glyburide), Glimepiride. The last two members of the list (Repaglinide, and Nateglinide) differ in their specific mechanism of action (Meglitinides), but again are oral agents that stimulate insulin secretion. The Buse reference focuses on studies that are directed at lipid effects, but also illustrates classes of compounds well known as “sulfonylureas”. For example, it is widely believed that only a few compounds constitute the major market share of “sulfonylureas,” such as Dymelor, Diabinese, Amaryl, Glucotrol, Micronase, Tolinase, Orinase and their generic equivalents (see pgs 725-32, chapter 41, Nolte M. S. and Karam, J. H., “Pancreatic Hormones & Antidiabetic Drugs” In Basic & Clinical Pharmacology, Katzung, B. G., Ed., Lange Medical Books, New York, 2001).

Examples of sulfonylureas and the insulin secretagogues known as glinides are Glipizide, Gliclazide, Glibenclamide (glyburide), Glimepiride, and the glinides Repaglinide, and Nateglinide).

The term “biguanides” refers to well known biguanides compounds, such as extensively reviewed on pages 716-20, chapter 41, Nolte M. S. and Karam, J. H., “Pancreatic Hormones & Antidiabetic Drugs” In Basic & Clinical Pharmacology, Katzung, B. G., Ed., Lange Medical Books, New York, 2001. For example, well known compounds that constitute the major market share of “biguanides” include metformin (Glucophage), buformin, and phenformin (Buse, J., et al. “The effects of oral anti-hyperglycaemic medications on serum lipidprofiles in patients with type 2 diabetes.” Diabetes Obesity Metabol. 6: 133-156 (2004)).

Examples of the “biguanides” are metformin (Glucophage), buformin, and phenformin.

The term “α-glucosidase inhibitors” refers to well known compounds having α-glucosidase inhibitors activity which has been the subject of extensive clinical studies (pg 729-30, chapter 41, Nolte M. S. and Karam, J. H., “Pancreatic Hormones & Antidiabetic Drugs” In Basic & Clinical Pharmacology, Katzung, B. G., Ed., Lange Medical Books, New York, 2001; Buse, J., et al. “The effects of oral anti-hyperglycaemic medications on serum lipid profiles in patients with type 2 diabetes.” Diabetes Obesity Metabol. 6: 133-156 (2004)). Compounds that constitute the major market share of “α-glucosidase inhibitors” include acarbose (Precose) and miglitol (Glycet).

Examples of “α-glucosidase inhibitors” are acarbose (Precose) and miglitol (Glycet).

The term “PPAR ligands” refers to compounds having Peroxisome Proliferator-Activated Receptor Ligand activity, also interchangeably referred to as thizolidinediones for the predominant structural class, as compounds active in the treatment of type 2 diabetes (c.f. pg 728, chapter 41, Nolte M. S. and Karam, J. H., “Pancreatic Hormones & Antidiabetic Drugs” In Basic & Clinical Pharmacology, Katzung, B. G., Ed., Lange Medical Books, New York, 2001; Lee, et al. “Minireview. Lipid Metabolism, Metabolic Diseases, and Peroxisome Proliferator-Activated Receptors”. Endocrinol. 144: 2201-7 (2003)). PPAR ligands such as pioglitazone are known to have beneficial effects on protection of pancreatic islets (Diani, A. R., et al. “Pioglitazone preserves pancreatic islet structure and insulin secretoryfunction in three murine models of type 2 diabetes”. Am. J. Physiol. Endocrinol. Metab. 286: E116-122 (2004). Compounds that constitute the major market share of “PPAR ligands” include pioglitizone (Actos) and rosiglitazone (Avandia) (c.f. pg 732 in Nolte, M. S. and Karam, J. H. 2001, referenced above). Additional PPAR ligands are undergoing clinical trials.

Examples of currently marketed PPARγ pharmaceuticals are the thiazolidinediones pioglitizone (Actos) and rosiglitazone (Avandia).

The term DPPIV inhibitor refers to compounds that that are intended to potentiate the endogenous incretin response by preventing the proteolysis of GLP1 or GIP through the inhibition of one or more of the DPPIV isoforms in the body (McIntosh, C. H. S., et al., Regulatory Peptides 128: 159-65 (2005)). A number of such agents are in review at the FDA or in clinical development (Hunziker, D., et al., Curr. Top. Med. Chem. 5: 1623-37 (2005); Kim, D., et al., J. Med. Chem. 48: 141-51 (2005)), Some non-limiting examples of such agents are: Galvus (vildagliptin; LAF 237); Januvia (sitagliptin; MK-431); saxagliptin; sulphostin; “P93/01”; “KRP-104”; “PHX1149” (Phenomix Corp); and the like.

For combination treatment with more than one active agent, where the active agents are in separate dosage formulations, the active agents can be administered concurrently, or they each can be administered at separately staggered times.

The dosages of the compounds of the present invention are adjusted when combined with other therapeutic agents. Dosages of these various agents may be independently optimized and combined to achieve a synergistic result wherein the pathology is reduced more than it would be if either agent were used alone. In addition, co-administration or sequential administration of other agents may be desirable.

In other contemplated disease applications, the peptides described herein can be used advantageously in coordination with pharmaceuticals currently applied for that disease. Particularly beneficial are combination drug formulations containing mixtures of the active pharmaceutical ingredients with excipients. For example, in asthma and COPD, the VPAC2 agonists can used in combination with inhaled formulations containing bronchodilators, β2 adrenoceptor agonists such as salmeterol, terbutaline, albuterol, bitolterol, pirbuterol, salbutamol, formoterol, indacaterol and the like (Sears, M. R and Lotvall, J., Resp. Med. 99: 152-170 (2005)); inhaled corticosteroids such as fluticasone (Flovent), budesonide (Pulmicort), triamcinolone acetonide, beclomethasone, flunisolide, ciclesonide, mometasone and the like; anti-inflammatory steroids; leukotriene modifiers; leukotriene receptor antagonists such as zafirlukast (Accolate®) and montelukast (Singulair®); 5-lipooxygenase inhibitors like zileuton; chemokine modifiers; chemokine receptor antagonists; cromolyn; nedocromil; xanthines such as theophylline; anticholinergic agents; immune modulating agents; protease inhibitors; other known anti-asthma medications, and the like. We expect that the additional agents in development (Corry D B and Kheradmand F (2006) J Allergy Clin Immunol 117 (2 Suppl): S461-47) also will be beneficial when used in combination with VPAC2 agonists.

VPAC2 combination treatments may make use of currently applied therapeutics for treatment of pulmonary hypertension, as well. Thus a VPAC2 agonist may be utilized in combination with nitric oxide donors, prostacyclins, endothelin antagonists, adrenoceptor blockers, phosphodiesterases inhibitors, ion channel blockers and other vasodilators (as outlined in Levy J H Tex Heart Inst J 32: 467-71 (2005); Haj R M, et al., Curr Opin Anesthesiol 19: 88-95 (2006)).

Non-limiting examples of particularly important classes of combination treatments for diabetes are VPAC2 Modulator plus Insulin Analog and VPAC2 Modulator plus Incretin Analog. Since PACAP and the “incretins” are complementary parts of the pancreatic beta cell response to a meal (neuronal and hormonal, respectively), use of the combination drug will be a more complete physiological mimic and may reduce the required dose of either, with expected beneficial effects. Specific, but non-limiting, examples here are BYETTA® (Amylin Pharmaceuticals, Inc., San Diego, Calif.) plus VPAC2 Modulator or liraglutide plus VPAC2 Modulator. Furthermore, being peptides of similar size, they can be delivered together from the same formulation. Similarly, insulin and the glucose-dependent insulin secretory response caused by the PACAP signal can be complementary and, importantly, lead to better glucose control with less risk of hypoglycemic responses. Specific, but non-limiting, examples here are Levemir plus VPAC2 Modulator or Lantus plus VPAC2 Modulator. Examples of combination treatments using DPPIV inhibitors are VPAC2 Modulator plus PHX1149 (Phenomix Corp), VPAC2 Modulator plus Galvus, or VPAC2 Modulator plus Januvia. Some DPPIV inhibitors have poor oral bioavailability and would benefit from a combination formulation for inhalation. In each of these instances the formulation and route of administration can be for use by injection or inhalation.

Similarly, important combination treatments for asthma are within the scope of the invention. Specific, but non-limiting, examples here relate to combinations with long-acting β2 adrenoceptor agonists such as: VPAC2 Modulator plus formoterol, VPAC2 Modulator plus indacaterol, and VPAC2 Modulator plus salmeterol. Another class of combination treatment uses inhaled corticosteroids with the VPAC2 Modulator. Non-limiting examples here are VPAC2 Modulator plus fluticasone, VPAC2 Modulator plus mometasone, VPAC2 Modulator plus beclomethasone, and VPAC2 Modulator plus Ciclesonide.

A particularly important consequence of such combination treatments is the potential for dose-sparing of these agents with their significant side effects, i.e. the insulin, incretin, β2 adreoceptor agonist, or corticosteroid analogs. This is particularly important in view of the severe nature of these side effects: for insulin, death from hypoglycemia; for incretin mimetics, emesis; for β2 adrenoceptor agonists, heart rate effects/sudden death; for corticosteroids, diminished growth in children. For the inhaled corticosteroids, the formulation of the agent with the very hydrophobic VPAC2 analog offers the further benefit of delayed release of the corticosteroid to prolong the relatively short duration of action of such agents (Winkler, J, et al., Proc Am Thorac Soc. 1: 356-63 (2004)). In each case the formulation of the combination treatment for inhalation offers significant commercial and medical benefits.

Representative delivery regimens include oral, parenteral (including subcutaneous, intramuscular and intravenous injection), rectal, buccal (including sublingual), transdermal, inhalation and intranasal. An attractive and widely used method for delivery of peptides entails subcutaneous injection of a controlled release injectable formulation. Preferred administration routes for the application of the peptides described herein are subcutaneous, intranasal and inhalation administration.

The selection of the exact dose and composition and the most appropriate delivery regimen will be influenced by, inter alia, the pharmacological properties of the selected polypeptide, the nature and severity of the condition being treated, and the physical condition and mental acuity of the recipient. Additionally, the route of administration will result in differential amounts of absorbed material. Bioavailabilities for administration of peptides through different routes are particularly variable, with amounts from less than 1% to near 100% being seen. Typically, bioavailability from routes other than intravenous injection are 50% or less.

In general, the polypeptides described herein, or salts thereof, are administered in amounts between about 0.1 and 60 μg/kg body weight per day, preferably from about 0.1 to about 1 μg/kg body weight per day, by subcutaneous injection. For a 50 kg human female subject, the daily dose of active ingredient is from about 5 to about 1000 μg, preferably from about 5 to about 500 μg by subcutaneous injection. Different doses will be needed, depending on the route of administration and the applicable bioavailability observed. By inhalation, the daily dose is from 100 to about 5,000 μg, twice daily. In other mammals, such as horses, dogs, and cattle, higher doses may be required. This dosage may be delivered in a conventional pharmaceutical composition by a single administration, by multiple applications, or via controlled release, as needed to achieve the most effective results, preferably one or more times daily by injection.

Pharmaceutically acceptable salts retain the desired biological activity of the parent polypeptide without toxic side effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, naphthalenesulfonic acids, naphthalene disulfonic acids, polygalacturonic acid and the like; (b) base addition salts formed with polyvalent metal cations such as zinc, calcium, bismuth, barium, magnesium, aluminum, copper, cobalt, nickel, cadmium, and the like; or with an organic cation formed from N,N′-dibenzylethylenediamine or ethylenediamine; or (c) combinations of (a) and (b), e.g., a zinc tannate salt and the like.

A further aspect of the present invention relates to pharmaceutical compositions comprising as an active ingredient a polypeptide of the present invention, or pharmaceutically acceptable salt thereof, in admixture with a pharmaceutically acceptable, non-toxic carrier. As mentioned above, such compositions may be prepared for parenteral (subcutaneous, intramuscular or intravenous) administration, particularly in the form of liquid solutions or suspensions; for oral or buccal administration, particularly in the form of tablets or capsules; for intranasal administration, particularly in the form of powders, nasal drops or aerosols; for inhalation, particularly in the form of liquid solutions or dry powders with excipients, defined broadly; and for rectal or transdermal administration.

The compositions may conveniently be administered in unit dosage form and may be prepared by any of the methods well-known in the pharmaceutical art, for example as described in Remington's Pharmaceutical Sciences, 17th ed., Mack Publishing Company, Easton, Pa., (1985), incorporated herein by reference. Formulations for parenteral administration may contain as excipients sterile water or saline, alkylene glycols such as propylene glycol, polyalkylene glycols such as polyethylene glycol, oils of vegetable origin, hydrogenated naphthalenes, serum albumin nanoparticles (as used in Abraxane™, American Pharmaceutical Partners, Inc. Schaumburg Ill.), and the like. For oral administration, the formulation can be enhanced by the addition of bile salts or acylcarnitines. Formulations for nasal administration may be solid and may contain excipients, for example, lactose or dextran, or may be aqueous or oily solutions for use in the form of nasal drops or metered spray. For buccal administration typical excipients include sugars, calcium stearate, magnesium stearate, pregelatinated starch, and the like.

When formulated for nasal administration, the absorption across the nasal mucous membrane may be enhanced by surfactant acids, such as for example, glycocholic acid, cholic acid, taurocholic acid, ethocholic acid, deoxycholic acid, chenodeoxycholic acid, dehydrocholic acid, glycodeoxycholic acid, cyclodextrins and the like in an amount in the range between about 0.2 and 15 weight percent, preferably between about 0.5 and 4 weight percent, most preferably about 2 weight percent. An additional class of absorption enhancers exhibiting greater efficacy with decreased irritation is the class of alkyl maltosides, such as tetradecylmaltoside (Arnold, J. J., et al., J. Pharm. Sci. 93, 2205-13 (2004) and references therein, all of which are hereby incorporated by reference).

When formulated for delivery by inhalation, a number of formulations offer advantages. Adsorption of the active peptide to readily dispersed solids such as diketopiperazines (for example Technosphere particles; Pfutzner, A. and Forst, T., Expert Opin Drug Deliv 2: 1097-106 (2005) or similar structures gives a formulation which results in a rapid initial uptake of the therapeutic agent. Lyophylized powders, especially glassy particles, containing the active peptide and an excipient are useful for delivery to the lung with good bioavailability, for example, see Exubera® (inhaled insulin by Pfizer and Aventis Pharmaceuticals Inc.). Additional systems for delivery of polypeptides by inhalation (Mandal, T. K., Am. J. Health Syst. Pharm. 62: 1359-64 (2005)) are well known in the art and are incorporated into this invention.

Delivery of the compounds of the present invention to the subject over prolonged periods of time, for example, for periods of one week to one year, may be accomplished by a single administration of a controlled release system containing sufficient active ingredient for the desired release period. Various controlled release systems, such as monolithic or reservoir-type microcapsules, depot implants, osmotic pumps, vesicles, micelles, liposomes, transdermal patches, iontophoretic devices and alternative injectable dosage forms may be utilized for this purpose. Localization at the site to which delivery of the active ingredient is desired is an additional feature of some controlled release devices, which may prove beneficial in the treatment of certain disorders.

One form of controlled release formulation contains the polypeptide or its salt dispersed or encapsulated in a slowly degrading, non-toxic, non-antigenic polymer such as copoly (lactic/glycolic) acid, as described in the pioneering work of Kent, Lewis, Sanders, and Tice, U.S. Pat. No. 4,675,189, incorporated by reference herein. The compounds or, preferably, their relatively insoluble salts, may also be formulated in cholesterol or other lipid matrix pellets, or silastomer matrix implants. Additional slow release, depot implant or injectable formulations will be apparent to the skilled artisan. See, for example, Sustained and Controlled Release Drug Delivery Systems, J. R. Robinson ed., Marcel Dekker, Inc., New York, 1978, and R. W. Baker, Controlled Release of Biologically Active Agents, John Wiley & Sons, New York, 1987, incorporated by reference herein.

All publications and patent applications mentioned in this specification are herein incorporated by reference to the same extent as if each independent publication or patent application is specifically and individually indicated to be incorporated by reference.

While the examples and discussion given above are intended to illustrate the synthesis and testing of representative compounds described herein, it will be understood that it is capable of further modifications and should not be construed as limiting the scope of the appended claims.

What is claimed is:

1. A vasoactive intestinal polypeptide selected from the group consisting of:
(a) a polypeptide or modified peptide comprising Formula C (SEQ ID NO: 81) or Formula D (SEQ ID NO: 424);
(b) a polypeptide selected from SEQ ID NO: 1 to SEQ ID NO: 66;
(c) a polypeptide selected from SEQ ID NO: 89 to SEQ ID NO: 315; and
(d) a polypeptide selected from SEQ ID NO: 319 to SEQ ID NO: 408.

2. The polypeptide of claim 1, wherein acyl is a C₄-C₉ acyl chain; long acyl is a C₆-C₂₀ acyl chain; and PEG is a polyethylene glycol chain of C₁₀₀-C₃₀₀₀ chain.

3. The polypeptide of claim 1, selected from the group consisting of SEQ ID NOs: 92, 112, 113, 117, 119, 120, 121, 123, 125, 127, 128, 132, 133, 134, 138, 139, 151, 152, 158, 160, 161, 164, 170, 172, 173, 174, 180 and 192.

4. The polypeptide of claim 1, selected from the group consisting of SEQ ID NO: 319 to SEQ ID NO: 348.

5. The polypeptide of claim 1, selected from the group consisting of SEQ ID NO: 349 to SEQ ID NO: 378.

6. The polypeptide of claim 1, selected from the group consisting of SEQ ID NO: 379 to SEQ ID NO: 408.

7. The polypeptide of claim 1, selected from the group consisting of SEQ ID NO: 89 to SEQ ID NO: 315.

8. The polypeptide of claim 1, selected from the group consisting of SEQ ID NO: 140, 142, 193, 195, 212, 240, 253, 255, 308, 329, 347, 359 and 389.

9. The polypeptides of claim 1 selected from the group consisting of 601, 603, 604, 605, 425-428, 430-433, 437-439, 441, 442, 445, 452, 455-457, 516, 550 and 551.

10. A method for producing the polypeptide of claim 1, said method comprising synthesizing the polypeptide by the sequential addition of protected amino acids to a peptide chain, removing the protecting groups, desalting and purifying the polypeptide.

11. The method of claim 8, further comprising the step of using microwave assistance.

12. A method for producing the polypeptide of claim 1, said method comprising:
(a) expressing a gene encoding said polypeptide;
(b) optionally purifying the expressed polypeptide;
(c) carrying out, on at least one amino acid of said polypeptide, at least one post expression modification selected from the group consisting of acylation, PEGylation, and combinations thereof, to provide at least one modified polypeptide; and
(d) purifying the modified polypeptide.

13. An expression vector encoding the polypeptide of claim 1.

14. A host cell transformed with an expression vector of claim 13.

15. A pharmaceutical composition comprising an effective amount of the polypeptide of claim 1, or acceptable salt thereof, and at least one pharmaceutically acceptable carrier or excipient.

16. The pharmaceutical composition of claim 15, further comprising an effective amount of at least one compound chosen from the group consisting of insulin, insulin analogs, incretin, incretin analogs, glucagon-like peptide, glucagon-like peptide analogs, glucose dependent insulinotropic peptide analogs, exendin, exendin analogs, DPPIV inhibitors, sulfonylureas, biguanides, α-glucosidase inhibitors, thiazolidinediones, peroxisome proliferator activated receptor (PPAR) agonists, PPAR antagonists and PPAR partial agonists.

17. A method of treating a disorder selected from elevated blood glucose levels, diabetes, insulin resistance, metabolic acidosis, obesity, asthma, chronic obstructive pulmonary disease, pulmonary hypertension, an inflammatory disease or a mammalian condition affected by VPAC receptor activation, the method comprising administering a therapeutically effective amount of the polypeptide of claim 1.

18. The method of claim 17, further comprising administering a therapeutically effective amount of at least one compound chosen from the group consisting of insulin, insulin analogs, incretin, incretin analogs, glucagon-like peptide, glucagon-like peptide analogs, glucose dependent insulinotropic peptide analogs, exendin, exendin analogs, DPPIV inhibitors, sulfonylureas, meglitinides, biguanides, α-glucosidase inhibitors, thiazolidinediones, PPAR agonists, PPAR antagonists and PPAR partial agonists.

19. The method of claim 17, wherein the diabetes is Type 2 diabetes mellitus.

20. The method of claim 17, wherein the asthma is the condition of bronchoconstriction.

21. The method of claim 20, further comprising administering a therapeutically effective amount of at least one compound chosen from the group consisting of inhaled formulations containing bronchodilators, β2 adrenoceptor agonists, inhaled corticosteroids, anti-inflammatory steroids, leukotriene modifiers, leukotriene receptor antagonists, chemokine modifiers, chemokine receptor antagonists, cromolyn, nedocromil, xanthines, anticholinergic agents, immune modulating agents, other known anti-asthma medications, phosphodiesterase inhibitors, other known anti-inflammatory medications and the like.

22. The method of claim 17, further comprising administering a therapeutically effective amount of at least one compound chosen from the group consisting of nitric oxide donors, prostacyclins, endothelin antagonists, adrenoceptor blockers, phosphodiesterases inhibitors, ion channel blockers, other known anti-inflammatory medications and other vasodilators.


SEQUENCE LISTING (605 sequences)

SEQ ID NO: 1 - protein, 38 aa, Artificial Sequence
  synthetic polypeptide
HSDAVFTDNYTRLRKQVAAKKYLNWIKKAKRELLEKLK

SEQ ID NO: 2 - protein, 42 aa, Artificial Sequence
  synthetic polypeptide
HSDAVFTDNYTRLRKQVAAKKYLNSIKKGKRELLEKLLRKLK

SEQ ID NO: 3 - protein, 38 aa, Artificial Sequence
  synthetic polypeptide
HSDAVFTDNYTRLRKQVAAKKYLNSIKKGKRELLEKLK

SEQ ID NO: 4 - protein, 42 aa, Artificial Sequence; identical to SEQ ID NO: 2
  synthetic polypeptide

SEQ ID NO: 5 - protein, 38 aa, Artificial Sequence
  synthetic polypeptide
HSDAVFTDNYTRLRKQVAAKKYLNDIKKGKRELLEKLK

SEQ ID NO: 6 - protein, 38 aa, Artificial Sequence; identical to SEQ ID NO: 1
  synthetic polypeptide

SEQ ID NO: 7 - protein, 41 aa, Artificial Sequence
  synthetic polypeptide
HSDAVFTDNYTRLRKQLAAKKYLNSIKKGKRLLRKLQPPPK

SEQ ID NO: 8 - protein, 41 aa, Artificial Sequence
  synthetic polypeptide
HSDAVFTDNYTRLRKQMAAKKYLNSIKKGKRELLEKLLRKL

SEQ ID NO: 9 - protein, 44 aa, Artificial Sequence
  synthetic polypeptide
HSDAVFTDNYTRLRKQMAAKKYLNSIKKGKRELLEKLLRKLPPP

SEQ ID NO: 10 - protein, 41 aa, Artificial Sequence
  synthetic polypeptide
HSDAVFTDNYTRLRKQMAAKKYLNSIKKGKRELLERLLRKK

SEQ ID NO: 11 - protein, 41 aa, Artificial Sequence; identical to SEQ ID NO: 8
  synthetic polypeptide

SEQ ID NO: 12 - protein, 41 aa, Artificial Sequence; identical to SEQ ID NO: 8
  synthetic polypeptide

SEQ ID NO: 13 - protein, 41 aa, Artificial Sequence
  synthetic polypeptide
HSDAVFTDNYTRLRKQMAAKKYLNSIKKGKRELLEKLLRKK

SEQ ID NO: 14 - protein, 44 aa, Artificial Sequence; identical to SEQ ID NO: 9
  synthetic polypeptide

SEQ ID NO: 15 - protein, 41 aa, Artificial Sequence
  synthetic polypeptide
HSDAVFTDNYTRLRKQMAAKKYLNSIKKGKRELLERLLRKL

SEQ ID NO: 16 - protein, 41 aa, Artificial Sequence; identical to SEQ ID NO: 13
  synthetic polypeptide

SEQ ID NO: 17 - protein, 44 aa, Artificial Sequence; identical to SEQ ID NO: 9
  synthetic polypeptide

SEQ ID NO: 18 - protein, 41 aa, Artificial Sequence; identical to SEQ ID NO: 15
  synthetic polypeptide

SEQ ID NO: 19 - protein, 41 aa, Artificial Sequence; identical to SEQ ID NO: 13
  synthetic polypeptide

SEQ ID NO: 20 - protein, 38 aa, Artificial Sequence
  synthetic polypeptide
HSDAVFTDNYTRLRKQVAAKKYLNSIKKAKRELLEKLK

SEQ ID NO: 21 - protein, 41 aa, Artificial Sequence
  synthetic polypeptide
HSDAVFTRNYTRLRRQLAARRYLNSIKKARRLLRRLLPPPK

SEQ ID NO: 22 - protein, 41 aa, Artificial Sequence
  synthetic polypeptide
HSDAVFTRNYTRLRRQLAARRYLNSIKKARRLLRRLQPPPK

SEQ ID NO: 23 - protein, 41 aa, Artificial Sequence; identical to SEQ ID NO: 7
  synthetic polypeptide

SEQ ID NO: 24 - protein, 38 aa, Artificial Sequence; identical to SEQ ID NO: 3
  synthetic polypeptide

SEQ ID NO: 25 - protein, 41 aa, Artificial Sequence
  synthetic polypeptide
HSDAVFTDNYTRLRKQVAAKKYLNSIKKGKRELLEKLKPPP

SEQ ID NO: 26 - protein, 41 aa, Artificial Sequence; identical to SEQ ID NO: 22
  synthetic polypeptide

SEQ ID NO: 27 - protein, 41 aa, Artificial Sequence
  synthetic polypeptide
HSDAVFTRNVTRLRRQLAARRVLNSIKKARRLLEKLLRKLK

SEQ ID NO: 28 - protein, 44 aa, Artificial Sequence
  synthetic polypeptide
HSDAVFTRNYTRLRRQLAARRYLNWIKKARRLLEKLLRKLKPPP

SEQ ID NO: 29 - protein, 42 aa, Artificial Sequence
  synthetic polypeptide
HSDAVFTRNYTRLRRQLAARRVLNWIKKARRELLEKLLRKLK

SEQ ID NO: 30 - protein, 40 aa, Artificial Sequence
  synthetic polypeptide
HSDAVFTRNYTRLRRQLAARRYLNSIKKARRLLEKLKPPP

SEQ ID NO: 31 - protein, 38 aa, Artificial Sequence
  synthetic polypeptide
HSDAVFTDNYTRLRKQLAAKKYLNWIKKAKRELLEKLK

SEQ ID NO: 32 - protein, 38 aa, Artificial Sequence; identical to SEQ ID NO: 20
  synthetic polypeptide

SEQ ID NO: 33 - protein, 38 aa, Artificial Sequence; identical to SEQ ID NO: 1
  synthetic polypeptide

SEQ ID NO: 34 - protein, 38 aa, Artificial Sequence; identical to SEQ ID NO: 1
  synthetic polypeptide

SEQ ID NO: 35 - protein, 38 aa, Artificial Sequence
  synthetic polypeptide
HSDAVFTDNYTRLRKQVAAKKYLNWIKRAKRELLEKLK

SEQ ID NO: 36 - protein, 41 aa, Artificial Sequence
  synthetic polypeptide
HSDAVFTDNYTRLRKQVAAKKYLNWIKKGKRLLRKLGPPPK

SEQ ID NO: 37 - protein, 41 aa, Artificial Sequence
  synthetic polypeptide
HSDAVFTDNYTRLRKQVAAKKYLNWIKKGKRLLRKLAPPPK

SEQ ID NO: 38 - protein, 41 aa, Artificial Sequence
  synthetic polypeptide
HSDAVFTDNYTRLRKQVAAKKYLNWIKKGKRLLRKLQPPPK

SEQ ID NO: 39 - protein, 41 aa, Artificial Sequence
  synthetic polypeptide
HSDAVFTDNYTRLRKQVAAKKYLNWIKKGKRLLRKLSPPPK

SEQ ID NO: 40 - protein, 38 aa, Artificial Sequence
  synthetic polypeptide
HSDAVFTDQYTRLLLKVAAKKYLQWIKKAKRELLEKLK

SEQ ID NO: 41 - protein, 38 aa, Artificial Sequence
  synthetic polypeptide
HSDAVFTDQYTRLLKQVAAKKYLQWIKKAKRELLEKLK

SEQ ID NO: 42 - protein, 41 aa, Artificial Sequence
  synthetic polypeptide
HSDAVFTDNYTRLRKQVAAKKYLNWIKKAKRLLRKLSPPPK

SEQ ID NO: 43 - protein, 38 aa, Artificial Sequence; identical to SEQ ID NO: 20
  synthetic polypeptide

SEQ ID NO: 44 - protein, 41 aa, Artificial Sequence; identical to SEQ ID NO: 21
  synthetic polypeptide

SEQ ID NO: 45 - protein, 41 aa, Artificial Sequence; identical to SEQ ID NO: 22
  synthetic polypeptide

SEQ ID NO: 46 - protein, 41 aa, Artificial Sequence; identical to SEQ ID NO: 7
  synthetic polypeptide

SEQ ID NO: 47 - protein, 38 aa, Artificial Sequence; identical to SEQ ID NO: 3
  synthetic polypeptide

SEQ ID NO: 48 - protein, 41 aa, Artificial Sequence; identical to SEQ ID NO: 25
  synthetic polypeptide

SEQ ID NO: 49 - protein, 41 aa, Artificial Sequence; identical to SEQ ID NO: 22
  synthetic polypeptide

SEQ ID NO: 50 - protein, 41 aa, Artificial Sequence
  synthetic polypeptide
HSDAVFTRNYTRLRRQLAARRYLNSIKKARRLLEKLLRKLK

SEQ ID NO: 51 - protein, 44 aa, Artificial Sequence; identical to SEQ ID NO: 28
  synthetic polypeptide

SEQ ID NO: 52 - protein, 42 aa, Artificial Sequence; identical to SEQ ID NO: 29
  synthetic polypeptide

SEQ ID NO: 53 - protein, 40 aa, Artificial Sequence; identical to SEQ ID NO: 30
  synthetic polypeptide

SEQ ID NO: 54 - protein, 41 aa, Artificial Sequence
  synthetic polypeptide
HSDAVFTDQYTRLRKQVAAKKYLQWIKKAKRLLRKLSPPPK

SEQ ID NO: 55 - protein, 41 aa, Artificial Sequence; identical to SEQ ID NO: 54
  synthetic polypeptide

SEQ ID NO: 56 - protein, 41 aa, Artificial Sequence
  synthetic polypeptide
HSDAVFTDNYTRLLRQVAARRYLNWIRRAKRLLRRLSPPPK

SEQ ID NO: 57 - protein, 41 aa, Artificial Sequence
  synthetic polypeptide
HSDAVFTDQVTRLLKQVAAKKYLQWIKKAKRLLRKLAPPPK

SEQ ID NO: 58 - protein, 41 aa, Artificial Sequence
  synthetic polypeptide
HSDAVFTDQYTRLLKQVAAKKYLQWIKKAKRLLRKLAPPPK

SEQ ID NO: 59 - protein, 41 aa, Artificial Sequence
  synthetic polypeptide
HSDAVFTDQYTRLLKQVAAKKYLQWIKKAKRLLRKLSPPPK

SEQ ID NO: 60 - protein, 38 aa, Artificial Sequence; identical to SEQ ID NO: 41
  synthetic polypeptide

SEQ ID NO: 61 - protein, 38 aa, Artificial Sequence
  synthetic polypeptide
HSDAVFTDQYTRLLRQVAARRYLQWIRRAKRELLEKLK

SEQ ID NO: 62 - protein, 38 aa, Artificial Sequence
  synthetic polypeptide
HSDAVFTDNYTRLLKQVAAKKYLNWIKKAKRELLEKLK

SEQ ID NO: 63 - protein, 38 aa, Artificial Sequence; identical to SEQ ID NO: 41
  synthetic polypeptide

SEQ ID NO: 64 - protein, 38 aa, Artificial Sequence; identical to SEQ ID NO: 40
  synthetic polypeptide

SEQ ID NO: 65 - protein, 38 aa, Artificial Sequence; identical to SEQ ID NO: 40
  synthetic polypeptide

SEQ ID NO: 66 - protein, 38 aa, Artificial Sequence
  synthetic polypeptide
HSDAVFTDQYTRLLKKVAAKKYLQWIKKAKRELLEKLK

SEQ ID NO: 67 - protein, 30 aa, Artificial Sequence
  synthetic polypeptide
HAEGTFTSDVSSYLEGQAAKEFIAWLVKGR

SEQ ID NO: 68 - protein, 39 aa, Artificial Sequence
  synthetic polypeptide
HGEGTFTSDLSKQMEEEAVRLFIEWLKNGGPSSGAPPPS

SEQ ID NO: 69 - protein, 44 aa, Artificial Sequence
  synthetic polypeptide
HGEGTFTSDLSKQMEEEAVRLFIEWLKNGGPSSGAPPSKKKKKK

SEQ ID NO: 70 - protein, 38 aa, Artificial Sequence
  synthetic polypeptide
HSDGIFTDSYSRYRKQMAVKKYLAAVLGKRYKQRVKNK

SEQ ID NO: 71 - protein, 38 aa, Artificial Sequence
  synthetic polypeptide
HSDAVFTDNYTRLRKQMAVKKYLNSILNGKRSSEGESP

SEQ ID NO: 72 - protein, 31 aa, Artificial Sequence
  synthetic polypeptide
HSDAVFTDNYTRLRKQVAAKKYLQSIKNKRY

SEQ ID NO: 73 - protein, 30 aa, Artificial Sequence
  synthetic polypeptide
HSDAVFTDNYTRLRKQMAAKKYLNSIKNKR

SEQ ID NO: 74 - protein, 30 aa, Artificial Sequence
  synthetic polypeptide
HSDAVFTDNYTRLRKQMAAKKYLNSIQNRR

SEQ ID NO: 75 - protein, 38 aa, Artificial Sequence
  synthetic polypeptide
HSDAVFTDNYTRLRKQMAAKKYLNSIKKGKRSSEGESP

SEQ ID NO: 76 - protein, 44 aa, Artificial Sequence
  synthetic polypeptide
HSDAVFTDNYTRLRKQVAAKKYLQSIKQKRYELLEKLLRKLRTA

SEQ ID NO: 77 - protein, 43 aa, Artificial Sequence
  synthetic polypeptide
HSDAVFTDNYTRLRKQVAAKKYLQSIKQKRELLEKLLRKLRTA

SEQ ID NO: 78 - protein, 35 aa, Artificial Sequence
  synthetic polypeptide
HSDAVFTDNYTRLRKQVAAKKYLQSILGSRTSPPP

SEQ ID NO: 79 - protein, 42 aa, Artificial Sequence
  synthetic polypeptide
YAEGTFISDYSIAMDKIHQQDFVNWLLAQKGKKNDWKHNTIQ

SEQ ID NO: 80 - protein, 35 aa, Artificial Sequence
  synthetic polypeptide
HSDAIFTQQYSKLLAKLALQKYLASILGSRTSPPP

SEQ ID NO: 81 - protein, 32 aa, Artificial Sequence
  synthetic peptide
HSDXXFTXXYXRXXXXXAXXXYLXXXXXXXXX

SEQ ID NO: 82 - protein, 8 aa, Artificial Sequence
  synthetic polypeptide
LLAKLALQ

SEQ ID NO: 83 - protein, 5 aa, Artificial Sequence
  synthetic peptide
XXXXX

SEQ ID NO: 84 - protein, 9 aa, Artificial Sequence
  synthetic peptide
XXXXXXXXX

SEQ ID NO: 85 - protein, 13 aa, Artificial Sequence
  synthetic peptide
XXXXXXXXXXXXX

SEQ ID NO: 86 - protein, 17 aa, Artificial Sequence
  synthetic peptide
XXXXXXXXXXXXXXXXX

SEQ ID NO: 87 - protein, 21 aa, Artificial Sequence
  synthetic peptide
XXXXXXXXXXXXXXXXXXXXX

SEQ ID NO: 88 - protein, 6 aa, Artificial Sequence
  synthetic peptide
XXXXXX

SEQ ID NO: 89 - protein, 41 aa, Artificial Sequence
  synthetic polypeptide
HSDAVFTDQYTRLLKQVAAKKYLQSIKKAKRLLRKLSPPPK

SEQ ID NO: 90 - protein, 41 aa, Artificial Sequence
  synthetic polypeptide
HSDAVFTDQYTRLRKQVAAKKYLQSIKNSRRLLRKLSPPPK

SEQ ID NO: 91 - protein, 41 aa, Artificial Sequence
  synthetic polypeptide
HSDAVFTDQYTKLLAKLAAKKYLQSIKNSRRLLRKLSPPPK

SEQ ID NO: 92 - protein, 41 aa, Artificial Sequence
  synthetic polypeptide
HSDAVFTDNYTRLLAKLALQKYLQSIKNKYRLLRKLSPPPK

SEQ ID NO: 93 - protein, 41 aa, Artificial Sequence
  synthetic polypeptide
HSDAVFTDNYTKLLAKLALQKYLQSIKNKYRLLRKLSPPPK

SEQ ID NO: 94 - protein, 41 aa, Artificial Sequence
  synthetic polypeptide
HSDAVFTDNYTKLLAKLALQKYLQSIKNKRYLLEKLSPPPK

SEQ ID NO: 95 - protein, 41 aa, Artificial Sequence
  synthetic polypeptide
HSDAVFTDNYTKLLAKLALQKYLQSIKNKRYLLRKLSPPPK

SEQ ID NO: 96 - protein, 37 aa, Artificial Sequence
  synthetic polypeptide
HSDAVFTDNYTKLLAKLALQKYLQSIKNKRYLLRKLK

SEQ ID NO: 97 - protein, 37 aa, Artificial Sequence
  synthetic polypeptide
HSDAVFTDNYTKLLKQLAAQKYLQSIKNKRYLLRKLK

SEQ ID NO: 98 - protein, 38 aa, Artificial Sequence
  synthetic polypeptide
HSDAVFTDQYTRLLKQVAAKKYLQWIKKAKRELLRKLK

SEQ ID NO: 99 - protein, 38 aa, Artificial Sequence
  synthetic polypeptide
HSDAVFTQQYTRLLKQVAAKKYLQWIKKAKRELLRKLK

SEQ ID NO: 100 - protein, 38 aa, Artificial Sequence
  synthetic polypeptide
HSDAVFTDQYTRLLAKVAAKKYLQWIKKAKRELLRKLK

SEQ ID NO: 101 - protein, 38 aa, Artificial Sequence
  synthetic polypeptide
HSDAVFTDQYTRLLKQVAAKKYLQSIKKAKRELLEKLK

SEQ ID NO: 102 - protein, 38 aa, Artificial Sequence
  synthetic polypeptide
HSDAVFTQQYTRLLKQVAAKKYLQWIKKAKRELLEKLK

SEQ ID NO: 103 - protein, 38 aa, Artificial Sequence
  synthetic polypeptide
HSDAVFTQNYTRLLKQVAAKKYLQSIKKAKRELLEKLK

SEQ ID NO: 104 - protein, 38 aa, Artificial Sequence
  synthetic polypeptide
HSDAVFTQNYTRLLKQVAAKKYLNSIKKAKRELLEKLK

SEQ ID NO: 105 - protein, 38 aa, Artificial Sequence
  synthetic polypeptide
HSDAVFTQNYTRLLKQVAAKKYLNSIKKAKRELLRKLK

SEQ ID NO: 106 - protein, 37 aa, Artificial Sequence
  synthetic polypeptide
HSDAVFTDNYTKLLAKLALQKYLQSIKNKRELLRKLK

SEQ ID NO: 107 - protein, 37 aa, Artificial Sequence
  synthetic polypeptide
HSDAVFTQNYTKLLAKLALQKYLQSIKNKRELLRKLK

SEQ ID NO: 108 - protein, 37 aa, Artificial Sequence
  synthetic polypeptide
HSDAVFTQNYTKLLAKLALQKYLQSIKNKRQLLRKLK

SEQ ID NO: 109 - protein, 37 aa, Artificial Sequence; identical to SEQ ID NO: 107
  synthetic polypeptide

SEQ ID NO: 110 - protein, 37 aa, Artificial Sequence
  synthetic polypeptide
HSDAVFTDNYTKLLAKLALQKYLQSIKNKARLLRKLK

SEQ ID NO: 111 - protein, 37 aa, Artificial Sequence
  synthetic polypeptide
HSDAVFTQNYTKLLAKLALQKYLQSIKNKARLLRKLK

SEQ ID NO: 112 - protein, 37 aa, Artificial Sequence
  synthetic polypeptide
HSDAVFTDNYTRLLKQVAAKKYLNSIKKAKRLLRKLK

SEQ ID NO: 113 - protein, 36 aa, Artificial Sequence
  synthetic polypeptide
HSDAVFTDNYTRLLKQVAAKKYLNSIKNKRLLRKLK

SEQ ID NO: 114 - protein, 37 aa, Artificial Sequence
  synthetic polypeptide
HSDAVFTDQYTRLLKQVAAKKYLQWIKKAKRLLKKLK

SEQ ID NO: 115 - protein, 37 aa, Artificial Sequence
  synthetic polypeptide
HSDAVFTDQYTRLLKQVAAKKYLQSIKKAKRLLKKLK

SEQ ID NO: 116 - protein, 37 aa, Artificial Sequence
  synthetic polypeptide
HSDAVFTDNYTRLLKQVAAKKYLNWIKKAKRLLKKLK

SEQ ID NO: 117 - protein, 37 aa, Artificial Sequence
  synthetic polypeptide
HSDAVFTDNYTRLLKQVAAKKYLNSIKKAKRLLKKLK

SEQ ID NO: 118 - protein, 37 aa, Artificial Sequence
  synthetic polypeptide
HSDAVFTDNYTRLFKQVAAKKYLNSIKKAKRLLKKLK

SEQ ID NO: 119 - protein, 37 aa, Artificial Sequence
  synthetic polypeptide
HSDAVFTQNYTRLLKQVAAKKYLNSIKKAKRLLKKLK

SEQ ID NO: 120 - protein, 37 aa, Artificial Sequence
  synthetic polypeptide
HSDAVFTQNYTRLLLKVAAKKYLNSIKKAKRLLKKLK

SEQ ID NO: 121 - protein, 37 aa, Artificial Sequence
  synthetic polypeptide
HSDAVFTDNYTRLLLKVAAKKYLNSIKKAKRLLKKLK

SEQ ID NO: 122 - protein, 37 aa, Artificial Sequence
  synthetic polypeptide
HSDAVFTENYTRLLLKVAAKKYLNSIKKAKRLLKKLK

SEQ ID NO: 123 - protein, 37 aa, Artificial Sequence
  synthetic polypeptide
HSDAVFTDNYTRLLAKVAAKKYLNSIKKAKRLLKKLK

SEQ ID NO: 124 - protein, 37 aa, Artificial Sequence
  synthetic polypeptide
HSDAVFTDNYTRLFAKVAAKKYLNSIKKAKRLLKKLK

SEQ ID NO: 125 - protein, 37 aa, Artificial Sequence
  synthetic polypeptide
HSDAVFTQQYTRLLKQVAAKKYLNSIKKAKRLLKKLK

SEQ ID NO: 126 - protein, 37 aa, Artificial Sequence
  synthetic polypeptide
HSDAVFTQQYTRLLAKVALKKYLNSIKKAKRLLKKLK

SEQ ID NO: 127 - protein, 37 aa, Artificial Sequence
  synthetic polypeptide
HSDAVFTQQYTRLLAKVALKKYLNSIKKAKELLKKLK

SEQ ID NO: 128 - protein, 37 aa, Artificial Sequence
  synthetic polypeptide
HSDAVFTQQYTRLLAKVALKKYLNSIKNAKRLLKKLK

SEQ ID NO: 129 - protein, 37 aa, Artificial Sequence; identical to SEQ ID NO: 128
  synthetic polypeptide

SEQ ID NO: 130 - protein, 37 aa, Artificial Sequence
  synthetic polypeptide
HSDAVFTQQYTRLLAKVALKKYLNSIQNAKRLLKKLK

SEQ ID NO: 131 - protein, 37 aa, Artificial Sequence
  synthetic polypeptide
HSDAVFTDQYTRLLAKVALKKYLNSIKNAKRLLKKLK

SEQ ID NO: 132 - protein, 37 aa, Artificial Sequence; identical to SEQ ID NO: 131
  synthetic polypeptide

SEQ ID NO: 133 - protein, 37 aa, Artificial Sequence
  synthetic polypeptide
HSDAVFTDQYTRLLKQVALKKYLNSIKNAKRLLKKLK

SEQ ID NO: 134 - protein, 37 aa, Artificial Sequence
  synthetic polypeptide
HSDAVFTQNYTRLRKQVAAKKYLNSIKKAKRLLKKLK

SEQ ID NO: 135 - protein, 37 aa, Artificial Sequence
  synthetic polypeptide
HSDAVFTQQYTRLRKQVAAKKYLNSIKKAKRLLKKLK

SEQ ID NO: 136 - protein, 37 aa, Artificial Sequence
  synthetic polypeptide
HSDAVFTDNYTRLRKQVAAKKYLNSIKKAKRLLKKLK

SEQ ID NO: 137 - protein, 37 aa, Artificial Sequence
  synthetic polypeptide
HSDAVFTENYTRLRKQVAAKKYLNSIKKAKRLLKKLK

SEQ ID NO: 138 - protein, 37 aa, Artificial Sequence
  synthetic polypeptide
HSDAVFTDNYTRLRKQVAAKKYLNSIKKAKELLKKLK

SEQ ID NO: 139 - protein, 37 aa, Artificial Sequence
  synthetic polypeptide
HSDAVFTQNYTRLRKQVAAKKYLNSIKKAKELLKKLK

SEQ ID NO: 140 - protein, 37 aa, Artificial Sequence
  synthetic polypeptide
HSDAVFTQQYTRLRKQVAAKKYLNSIKKAKELLKKLK

SEQ ID NO: 141 - protein, 37 aa, Artificial Sequence
  synthetic polypeptide
HSDAVFTQQYTRLRLQVALKKYLNSIKKAKRLLKKLK

SEQ ID NO: 142 - protein, 37 aa, Artificial Sequence
  synthetic polypeptide
HSDAVFTQQYTRLRKQVALKKYLNSIKKAKELLKKLK

SEQ ID NO: 143 - protein, 37 aa, Artificial Sequence
  synthetic polypeptide
HSDAVFTQQYTRLRKQVALKKYLNSIKNAKRLLKKLK

SEQ ID NO: 144 - protein, 37 aa, Artificial Sequence; identical to SEQ ID NO: 143
  synthetic polypeptide

SEQ ID NO: 145 - protein, 37 aa, Artificial Sequence
  synthetic polypeptide
HSDAVFTQQYTRLRKQVALKKYLNSIQNAKRLLKKLK

SEQ ID NO: 146 - protein, 37 aa, Artificial Sequence
  synthetic polypeptide
HSDAVFTDQYTRLRKQVALKKYLNSIKNAKRLLKKLK

SEQ ID NO: 147 - protein, 37 aa, Artificial Sequence; identical to SEQ ID NO: 146
  synthetic polypeptide

SEQ ID NO: 148 - protein, 37 aa, Artificial Sequence
  synthetic polypeptide
HSDAVFTDQYTRLRKQVALKKYLNSIKNAKRLLEKLK

SEQ ID NO: 149 - protein, 41 aa, Artificial Sequence; identical to SEQ ID NO: 95
  synthetic polypeptide

SEQ ID NO: 150 - protein, 41 aa, Artificial Sequence
  synthetic polypeptide
HSDAVFTDNYTKLLKQLAAQKYLQSIKNKRYLLRKLSPPPK

SEQ ID NO: 151 - protein, 42 aa, Artificial Sequence
  synthetic polypeptide
HSDAVFTDQYTRLLKQVAAKKYLQWIKKAKRELLRKLSPPPK

SEQ ID NO: 152 - protein, 42 aa, Artificial Sequence
  synthetic polypeptide
HSDAVFTQQYTRLLKQVAAKKYLQWIKKAKRELLRKLSPPPK

SEQ ID NO: 153 - protein, 42 aa, Artificial Sequence
  synthetic polypeptide
HSDAVFTDQYTRLLAKVAAKKYLQWIKKAKRELLRKLSPPPK

SEQ ID NO: 154 - protein, 42 aa, Artificial Sequence
  synthetic polypeptide
HSDAVFTDQYTRLLKQVAAKKYLQSIKKAKRELLEKLSPPPK

SEQ ID NO: 155 - protein, 42 aa, Artificial Sequence
  synthetic polypeptide
HSDAVFTQQYTRLLKQVAAKKYLQWIKKAKRELLEKLSPPPK

SEQ ID NO: 156 - protein, 42 aa, Artificial Sequence
  synthetic polypeptide
HSDAVFTQNYTRLLKQVAAKKYLQSIKKAKRELLEKLSPPPK

SEQ ID NO: 157 - protein, 42 aa, Artificial Sequence
  synthetic polypeptide
HSDAVFTQNYTRLLKQVAAKKYLNSIKKAKRELLEKLSPPPK

SEQ ID NO: 158 - protein, 42 aa, Artificial Sequence
  synthetic polypeptide
HSDAVFTQNYTRLLKQVAAKKYLNSIKKAKRELLRKLSPPPK

SEQ ID NO: 159 - protein, 41 aa, Artificial Sequence
  synthetic polypeptide
HSDAVFTDNYTKLLAKLALQKYLQSIKNKRELLRKLSPPPK

SEQ ID NO: 160 - protein, 41 aa, Artificial Sequence
  synthetic polypeptide
HSDAVFTQNYTKLLAKLALQKYLQSIKNKRELLRKLSPPPK

SEQ ID NO: 161 - protein, 41 aa, Artificial Sequence
  synthetic polypeptide
HSDAVFTQNYTKLLAKLALQKYLQSIKNKRQLLRKLSPPPK

SEQ ID NO: 162 - protein, 41 aa, Artificial Sequence; identical to SEQ ID NO: 160
  synthetic polypeptide

SEQ ID NO: 163 - protein, 41 aa, Artificial Sequence
  synthetic polypeptide
HSDAVFTDNYTKLLAKLALQKYLQSIKNKARLLRKLSPPPK

SEQ ID NO: 164 - protein, 41 aa, Artificial Sequence
  synthetic polypeptide
HSDAVFTQNYTKLLAKLALQKYLQSIKNKARLLRKLSPPPK

SEQ ID NO: 165 - protein, 41 aa, Artificial Sequence
  synthetic polypeptide
HSDAVFTDNYTRLLKQVAAKKYLNSIKKAKRLLRKLSPPPK

SEQ ID NO: 166 - protein, 40 aa, Artificial Sequence
  synthetic polypeptide
HSDAVFTDNYTRLLKQVAAKKYLNSIKNKRLLRKLSPPPK

SEQ ID NO: 167 - protein, 41 aa, Artificial Sequence
  synthetic polypeptide
HSDAVFTDQYTRLLKQVAAKKYLQWIKKAKRLLKKLSPPPK

SEQ ID NO: 168 - protein, 41 aa, Artificial Sequence
  synthetic polypeptide
HSDAVFTDQYTRLLKQVAAKKYLQSIKKAKRLLKKLSPPPK

SEQ ID NO: 169 - protein, 41 aa, Artificial Sequence
  synthetic polypeptide
HSDAVFTDNYTRLLKQVAAKKYLNWIKKAKRLLKKLSPPPK

SEQ ID NO: 170 - protein, 41 aa, Artificial Sequence
  synthetic polypeptide
HSDAVFTDNYTRLLKQVAAKKYLNSIKKAKRLLKKLSPPPK

SEQ ID NO: 171 - protein, 41 aa, Artificial Sequence
  synthetic polypeptide
HSDAVFTDNYTRLFKQVAAKKYLNSIKKAKRLLKKLSPPPK

SEQ ID NO: 172 - protein, 41 aa, Artificial Sequence
  synthetic polypeptide
HSDAVFTQNYTRLLKQVAAKKYLNSIKKAKRLLKKLSPPPK

SEQ ID NO: 173 - protein, 41 aa, Artificial Sequence
  synthetic polypeptide
HSDAVFTQNYTRLLLKVAAKKYLNSIKKAKRLLKKLSPPPK

SEQ ID NO: 174 - protein, 41 aa, Artificial Sequence
  synthetic polypeptide
HSDAVFTDNYTRLLLKVAAKKYLNSIKKAKRLLKKLSPPPK

SEQ ID NO: 175 - protein, 41 aa, Artificial Sequence
  synthetic polypeptide
HSDAVFTENYTRLLLKVAAKKYLNSIKKAKRLLKKLSPPPK

SEQ ID NO: 176 - protein, 41 aa, Artificial Sequence
  synthetic polypeptide
HSDAVFTDNYTRLLAKVAAKKYLNSIKKAKRLLKKLSPPPK

SEQ ID NO: 177 - protein, 41 aa, Artificial Sequence
  synthetic polypeptide
HSDAVFTDNYTRLFAKVAAKKYLNSIKKAKRLLKKLSPPPK

SEQ ID NO: 178 - protein, 41 aa, Artificial Sequence
  synthetic polypeptide
HSDAVFTQQYTRLLKQVAAKKYLNSIKKAKRLLKKLSPPPK

SEQ ID NO: 179 - protein, 41 aa, Artificial Sequence
  synthetic polypeptide
HSDAVFTQQYTRLLAKVALKKYLNSIKKAKRLLKKLSPPPK

SEQ ID NO: 180 - protein, 41 aa, Artificial Sequence
  synthetic polypeptide
HSDAVFTQQYTRLLAKVALKKYLNSIKKAKELLKKLSPPPK

SEQ ID NO: 181 - protein, 41 aa, Artificial Sequence
  synthetic polypeptide
HSDAVFTQQYTRLLAKVALKKYLNSIKNAKRLLKKLSPPPK

SEQ ID NO: 182 - protein, 41 aa, Artificial Sequence; identical to SEQ ID NO: 181
  synthetic polypeptide

SEQ ID NO: 183 - protein, 41 aa, Artificial Sequence
  synthetic polypeptide
HSDAVFTQQYTRLLAKVALKKYLNSIQNAKRLLKKLSPPPK

SEQ ID NO: 184 - protein, 41 aa, Artificial Sequence
  synthetic polypeptide
HSDAVFTDQYTRLLAKVALKKYLNSIKNAKRLLKKLSPPPK

SEQ ID NO: 185 - protein, 41 aa, Artificial Sequence; identical to SEQ ID NO: 184
  synthetic polypeptide

SEQ ID NO: 186 - protein, 41 aa, Artificial Sequence
  synthetic polypeptide
HSDAVFTDQYTRLLKQVALKKYLNSIKNAKRLLKKLSPPPK

SEQ ID NO: 187 - protein, 41 aa, Artificial Sequence
  synthetic polypeptide
HSDAVFTQNYTRLRKQVAAKKYLNSIKKAKRLLKKLSPPPK

SEQ ID NO: 188 - protein, 41 aa, Artificial Sequence
  synthetic polypeptide
HSDAVFTQQYTRLRKQVAAKKYLNSIKKAKRLLKKLSPPPK

SEQ ID NO: 189 - protein, 41 aa, Artificial Sequence
  synthetic polypeptide
HSDAVFTDNYTRLRKQVAAKKYLNSIKKAKRLLKKLSPPPK

SEQ ID NO: 190 - protein, 41 aa, Artificial Sequence
  synthetic polypeptide
HSDAVFTENYTRLRKQVAAKKYLNSIKKAKRLLKKLSPPPK

SEQ ID NO: 191 - protein, 41 aa, Artificial Sequence
  synthetic polypeptide
HSDAVFTDNYTRLRKQVAAKKYLNSIKKAKELLKKLSPPPK

SEQ ID NO: 192 - protein, 41 aa, Artificial Sequence
  synthetic polypeptide
HSDAVFTQNYTRLRKQVAAKKYLNSIKKAKELLKKLSPPPK

SEQ ID NO: 193 - protein, 41 aa, Artificial Sequence
  synthetic polypeptide
HSDAVFTQQYTRLRKQVAAKKYLNSIKKAKELLKKLSPPPK

SEQ ID NO: 194 - protein, 41 aa, Artificial Sequence
  synthetic polypeptide
HSDAVFTQQYTRLRLQVALKKYLNSIKKAKRLLKKLSPPPK

SEQ ID NO: 195 - protein, 41 aa, Artificial Sequence
  synthetic polypeptide
HSDAVFTQQYTRLRKQVALKKYLNSIKKAKELLKKLSPPPK

SEQ ID NO: 196 - protein, 41 aa, Artificial Sequence
  synthetic polypeptide
HSDAVFTQQYTRLRKQVALKKYLNSIKNAKRLLKKLSPPPK

SEQ ID NO: 197 - protein, 41 aa, Artificial Sequence; identical to SEQ ID NO: 196
  synthetic polypeptide

SEQ ID NO: 198 - protein, 41 aa, Artificial Sequence
  synthetic polypeptide
HSDAVFTQQYTRLRKQVALKKYLNSIQNAKRLLKKLSPPPK

SEQ ID NO: 199 - protein, 41 aa, Artificial Sequence
  synthetic polypeptide
HSDAVFTDQYTRLRKQVALKKYLNSIKNAKRLLKKLSPPPK

SEQ ID NO: 200 - protein, 41 aa, Artificial Sequence; identical to SEQ ID NO: 199
  synthetic polypeptide

SEQ ID NO: 201 - protein, 41 aa, Artificial Sequence
  synthetic polypeptide
HSDAVFTDQYTRLRKQVALKKYLNSIKNAKRLLEKLSPPPK

SEQ ID NO: 202 - protein, 42 aa, Artificial Sequence
  synthetic polypeptide
HSDAVFTDQYTRLLKQVAAKKYLQSIKKAKRLLRKLSPPPKC

SEQ ID NO: 203 - protein, 42 aa, Artificial Sequence
  synthetic polypeptide
HSDAVFTDQYTRLRKQVAAKKYLQSIKNSRRLLRKLSPPPKC

SEQ ID NO: 204 - protein, 42 aa, Artificial Sequence
  synthetic polypeptide
HSDAVFTDQYTKLLAKLAAKKYLQSIKNSRRLLRKLSPPPKC

SEQ ID NO: 205 - protein, 42 aa, Artificial Sequence
  synthetic polypeptide
HSDAVFTDNYTRLLAKLALQKYLQSIKNKYRLLRKLSPPPKC

SEQ ID NO: 206 - protein, 42 aa, Artificial Sequence
  synthetic polypeptide
HSDAVFTDNYTKLLAKLALQKYLQSIKNKYRLLRKLSPPPKC

SEQ ID NO: 207 - protein, 42 aa, Artificial Sequence
  synthetic polypeptide
HSDAVFTDNYTKLLAKLALQKYLQSIKNKRYLLEKLSPPPKC

SEQ ID NO: 208 - protein, 42 aa, Artificial Sequence
  synthetic polypeptide
HSDAVFTDNYTKLLAKLALQKYLQSIKNKRYLLRKLSPPPKC

SEQ ID NO: 209 - protein, 38 aa, Artificial Sequence
  synthetic polypeptide
HSDAVFTDNYTKLLAKLALQKYLQSIKNKRYLLRKLKC

SEQ ID NO: 210 - protein, 38 aa, Artificial Sequence
  synthetic polypeptide
HSDAVFTDNYTKLLKQLAAQKYLQSIKNKRYLLRKLKC

SEQ ID NO: 211 - protein, 39 aa, Artificial Sequence
  synthetic polypeptide
HSDAVFTDQYTRLLKQVAAKKYLQWIKKAKRELLRKLKC

SEQ ID NO: 212 - protein, 39 aa, Artificial Sequence
  synthetic polypeptide
HSDAVFTQQYTRLLKQVAAKKYLQWIKKAKRELLRKLKC

SEQ ID NO: 213 - protein, 39 aa, Artificial Sequence
  synthetic polypeptide
HSDAVFTDQYTRLLAKVAAKKYLQWIKKAKRELLRKLKC

SEQ ID NO: 214 - protein, 39 aa, Artificial Sequence
  synthetic polypeptide
HSDAVFTDQYTRLLKQVAAKKYLQSIKKAKRELLEKLKC

SEQ ID NO: 215 - protein, 39 aa, Artificial Sequence
  synthetic polypeptide
HSDAVFTQQYTRLLKQVAAKKYLQWIKKAKRELLEKLKC

SEQ ID NO: 216 - protein, 39 aa, Artificial Sequence
  synthetic polypeptide
HSDAVFTQNYTRLLKQVAAKKYLQSIKKAKRELLEKLKC

SEQ ID NO: 217 - protein, 39 aa, Artificial Sequence
  synthetic polypeptide
HSDAVFTQNYTRLLKQVAAKKYLNSIKKAKRELLEKLKC

SEQ ID NO: 218 - protein, 39 aa, Artificial Sequence
  synthetic polypeptide
HSDAVFTQNYTRLLKQVAAKKYLNSIKKAKRELLRKLKC

SEQ ID NO: 219 - protein, 38 aa, Artificial Sequence
  synthetic polypeptide
HSDAVFTDNYTKLLAKLALQKYLQSIKNKRELLRKLKC

SEQ ID NO: 220 - protein, 38 aa, Artificial Sequence
  synthetic polypeptide
HSDAVFTQNYTKLLAKLALQKYLQSIKNKRELLRKLKC

SEQ ID NO: 221 - protein, 38 aa, Artificial Sequence
  synthetic polypeptide
HSDAVFTQNYTKLLAKLALQKYLQSIKNKRQLLRKLKC

SEQ ID NO: 222 - protein, 38 aa, Artificial Sequence; identical to SEQ ID NO: 220
  synthetic polypeptide

SEQ ID NO: 223 - protein, 38 aa, Artificial Sequence
  synthetic polypeptide
HSDAVFTDNYTKLLAKLALQKYLQSIKNKARLLRKLKC

SEQ ID NO: 224 - protein, 38 aa, Artificial Sequence
  synthetic polypeptide
HSDAVFTQNYTKLLAKLALQKYLQSIKNKARLLRKLKC

SEQ ID NO: 225 - protein, 39 aa, Artificial Sequence
  synthetic polypeptide
HSDAVFTDNYTRLLKQVAAKKYLNSIKKAKRLLRKLSKC

SEQ ID NO: 226 - protein, 37 aa, Artificial Sequence
  synthetic polypeptide
HSDAVFTDNYTRLLKQVAAKKYLNSIKNKRLLRKLKC

SEQ ID NO: 227 - protein, 38 aa, Artificial Sequence
  synthetic polypeptide
HSDAVFTDQYTRLLKQVAAKKYLQWIKKAKRLLKKLKC

SEQ ID NO: 228 - protein, 38 aa, Artificial Sequence
  synthetic polypeptide
HSDAVFTDQYTRLLKQVAAKKYLQSIKKAKRLLKKLKC

SEQ ID NO: 229 - protein, 38 aa, Artificial Sequence
  synthetic polypeptide
HSDAVFTDNYTRLLKQVAAKKYLNWIKKAKRLLKKLKC

SEQ ID NO: 230 - protein, 38 aa, Artificial Sequence
  synthetic polypeptide
HSDAVFTDNYTRLLKQVAAKKYLNSIKKAKRLLKKLKC

SEQ ID NO: 231 - protein, 38 aa, Artificial Sequence
  synthetic polypeptide
HSDAVFTDNYTRLFKQVAAKKYLNSIKKAKRLLKKLKC

SEQ ID NO: 232 - protein, 38 aa, Artificial Sequence
  synthetic polypeptide
HSDAVFTQNYTRLLKQVAAKKYLNSIKKAKRLLKKLKC

SEQ ID NO: 233 - protein, 38 aa, Artificial Sequence
  synthetic polypeptide
HSDAVFTQNYTRLLLKVAAKKYLNSIKKAKRLLKKLKC

SEQ ID NO: 234 - protein, 38 aa, Artificial Sequence
  synthetic polypeptide
HSDAVFTDNYTRLLLKVAAKKYLNSIKKAKRLLKKLKC

SEQ ID NO: 235 - protein, 38 aa, Artificial Sequence
  synthetic polypeptide
HSDAVFTENYTRLLLKVAAKKYLNSIKKAKRLLKKLKC

SEQ ID NO: 236 - protein, 38 aa, Artificial Sequence
  synthetic polypeptide
HSDAVFTDNYTRLLAKVAAKKYLNSIKKAKRLLKKLKC

SEQ ID NO: 237 - protein, 38 aa, Artificial Sequence
  synthetic polypeptide
HSDAVFTDNYTRLFAKVAAKKYLNSIKKAKRLLKKLKC

SEQ ID NO: 238 - protein, 38 aa, Artificial Sequence
  synthetic polypeptide
HSDAVFTQQYTRLLKQVAAKKYLNSIKKAKRLLKKLKC

SEQ ID NO: 239 - protein, 38 aa, Artificial Sequence
  synthetic polypeptide
HSDAVFTQQYTRLLAKVALKKYLNSIKKAKRLLKKLKC

SEQ ID NO: 240 - protein, 38 aa, Artificial Sequence
  synthetic polypeptide
HSDAVFTQQYTRLLAKVALKKYLNSIKKAKELLKKLKC

SEQ ID NO: 241 - protein, 38 aa, Artificial Sequence
  synthetic polypeptide
HSDAVFTQQYTRLLAKVALKKYLNSIKNAKRLLKKLKC

SEQ ID NO: 242 - protein, 38 aa, Artificial Sequence; identical to SEQ ID NO: 241
  synthetic polypeptide

SEQ ID NO: 243 - protein, 38 aa, Artificial Sequence
  synthetic polypeptide
HSDAVFTQQYTRLLAKVALKKYLNSIQNAKRLLKKLKC

SEQ ID NO: 244 - protein, 38 aa, Artificial Sequence
  synthetic polypeptide
HSDAVFTDQYTRLLAKVALKKYLNSIKNAKRLLKKLKC

SEQ ID NO: 245 - protein, 38 aa, Artificial Sequence; identical to SEQ ID NO: 244
  synthetic polypeptide

SEQ ID NO: 246 - protein, 38 aa, Artificial Sequence
  synthetic polypeptide
HSDAVFTDQYTRLLKQVALKKYLNSIKNAKRLLKKLKC

SEQ ID NO: 247 - protein, 38 aa, Artificial Sequence
  synthetic polypeptide
HSDAVFTQNYTRLRKQVAAKKYLNSIKKAKRLLKKLKC

SEQ ID NO: 248 - protein, 38 aa, Artificial Sequence
  synthetic polypeptide
HSDAVFTQQYTRLRKQVAAKKYLNSIKKAKRLLKKLKC

SEQ ID NO: 249 - protein, 38 aa, Artificial Sequence
  synthetic polypeptide
HSDAVFTDNYTRLRKQVAAKKYLNSIKKAKRLLKKLKC

SEQ ID NO: 250 - protein, 37 aa, Artificial Sequence
  synthetic polypeptide
HSDAVFTENYTRLRKQVAKKYLNSIKKAKRLLKKLKC

SEQ ID NO: 251 - protein, 38 aa, Artificial Sequence
  synthetic polypeptide
HSDAVFTDNYTRLRKQVAAKKYLNSIKKAKELLKKLKC

SEQ ID NO: 252 - protein, 38 aa, Artificial Sequence
  synthetic polypeptide
HSDAVFTQNYTRLRKQVAAKKYLNSIKKAKELLKKLKC

SEQ ID NO: 253 - protein, 38 aa, Artificial Sequence
  synthetic polypeptide
HSDAVFTQQYTRLRKQVAAKKYLNSIKKAKELLKKLKC

SEQ ID NO: 254 - protein, 38 aa, Artificial Sequence
  synthetic polypeptide
HSDAVFTQQYTRLRLQVALKKYLNSIKKAKRLLKKLKC

SEQ ID NO: 255 - protein, 38 aa, Artificial Sequence
  synthetic polypeptide
HSDAVFTQQYTRLRKQVALKKYLNSIKKAKELLKKLKC

SEQ ID NO: 256 - protein, 38 aa, Artificial Sequence
  synthetic polypeptide
HSDAVFTQQYTRLRKQVALKKYLNSIKNAKRLLKKLKC

SEQ ID NO: 257 - protein, 38 aa, Artificial Sequence; identical to SEQ ID NO: 256
  synthetic polypeptide

SEQ ID NO: 258 - protein, 38 aa, Artificial Sequence
  synthetic polypeptide
HSDAVFTQQYTRLRKQVALKKYLNSIQNAKRLLKKLKC

SEQ ID NO: 259 - protein, 38 aa, Artificial Sequence
  synthetic polypeptide
HSDAVFTDQYTRLRKQVALKKYLNSIKNAKRLLKKLKC

SEQ ID NO: 260 - protein, 38 aa, Artificial Sequence; identical to SEQ ID NO: 259
  synthetic polypeptide

SEQ ID NO: 261 - protein, 38 aa, Artificial Sequence; identical to SEQ ID NO: 259
  synthetic polypeptide

SEQ ID NO: 262 - protein, 42 aa, Artificial Sequence; identical to SEQ ID NO: 208
  synthetic polypeptide

SEQ ID NO: 263 - protein, 42 aa, Artificial Sequence
  synthetic polypeptide
HSDAVFTDNYTKLLKQLAAQKYLQSIKNKRYLLRKLSPPPKC

SEQ ID NO: 264 - protein, 43 aa, Artificial Sequence
  synthetic polypeptide
HSDAVFTDQYTRLLKQVAAKKYLQWIKKAKRELLRKLSPPPKC

SEQ ID NO: 265 - protein, 43 aa, Artificial Sequence
  synthetic polypeptide
HSDAVFTQQYTRLLKQVAAKKYLQWIKKAKRELLRKLSPPPKC

SEQ ID NO: 266 - protein, 43 aa, Artificial Sequence
  synthetic polypeptide
HSDAVFTDQYTRLLAKVAAKKYLQWIKKAKRELLRKLSPPPKC

SEQ ID NO: 267 - protein, 43 aa, Artificial Sequence
  synthetic polypeptide
HSDAVFTDQYTRLLKQVAAKKYLQSIKKAKRELLEKLSPPPKC

SEQ ID NO: 268 - protein, 43 aa, Artificial Sequence
  synthetic polypeptide
HSDAVFTQQYTRLLKQVAAKKYLQWIKKAKRELLEKLSPPPKC

SEQ ID NO: 269 - protein, 43 aa, Artificial Sequence
  synthetic polypeptide
HSDAVFTQNYTRLLKQVAAKKYLQSIKKAKRELLEKLSPPPKC

SEQ ID NO: 270 - protein, 43 aa, Artificial Sequence
  synthetic polypeptide
HSDAVFTQNYTRLLKQVAAKKYLNSIKKAKRELLEKLSPPPKC

SEQ ID NO: 271 - protein, 43 aa, Artificial Sequence
  synthetic polypeptide
HSDAVFTQNYTRLLKQVAAKKYLNSIKKAKRELLRKLSPPPKC

SEQ ID NO: 272 - protein, 42 aa, Artificial Sequence
  synthetic polypeptide
HSDAVFTDNYTKLLAKLALQKYLQSIKNKRELLRKLSPPPKC

SEQ ID NO: 273 - protein, 42 aa, Artificial Sequence
  synthetic polypeptide
HSDAVFTQNYTKLLAKLALQKYLQSIKNKRELLRKLSPPPKC

SEQ ID NO: 274 - protein, 42 aa, Artificial Sequence
  synthetic polypeptide
HSDAVFTQNYTKLLAKLALQKYLQSIKNKRQLLRKLSPPPKC

SEQ ID NO: 275 - protein, 42 aa, Artificial Sequence; identical to SEQ ID NO: 273
  synthetic polypeptide

SEQ ID NO: 276 - protein, 42 aa, Artificial Sequence
  synthetic polypeptide
HSDAVFTDNYTKLLAKLALQKYLQSIKNKARLLRKLSPPPKC

SEQ ID NO: 277 - protein, 42 aa, Artificial Sequence
  synthetic polypeptide
HSDAVFTQNYTKLLAKLALQKYLQSIKNKARLLRKLSPPPKC

SEQ ID NO: 278 - protein, 42 aa, Artificial Sequence
  synthetic polypeptide
HSDAVFTDNYTRLLKQVAAKKYLNSIKKAKRLLRKLSPPPKC

SEQ ID NO: 279 - protein, 42 aa, Artificial Sequence
  synthetic polypeptide
HSDAVFTDNYTRLLKQVAAKKYLNSIKNKRLLRKLSSPPPKC

SEQ ID NO: 280 - protein, 42 aa, Artificial Sequence
  synthetic polypeptide
HSDAVFTDQYTRLLKQVAAKKYLQWIKKAKRLLKKLSPPPKC

SEQ ID NO: 281 - protein, 42 aa, Artificial Sequence
  synthetic polypeptide
HSDAVFTDQYTRLLKQVAAKKYLQSIKKAKRLLKKLSPPPKC

SEQ ID NO: 282 - protein, 42 aa, Artificial Sequence
  synthetic polypeptide
HSDAVFTDNYTRLLKQVAAKKYLNWIKKAKRLLKKLSPPPKC

SEQ ID NO: 283 - protein, 42 aa, Artificial Sequence
  synthetic polypeptide
HSDAVFTDNYTRLLKQVAAKKYLNSIKKAKRLLKKLSPPPKC

SEQ ID NO: 284 - protein, 42 aa, Artificial Sequence
  synthetic polypeptide
HSDAVFTDNYTRLFKQVAAKKYLNSIKKAKRLLKKLSPPPKC

SEQ ID NO: 285 - protein, 42 aa, Artificial Sequence
  synthetic polypeptide
HSDAVFTQNYTRLLKQVAAKKYLNSIKKAKRLLKKLSPPPKC

SEQ ID NO: 286 - protein, 42 aa, Artificial Sequence
  synthetic polypeptide
HSDAVFTQNYTRLLLKVAAKKYLNSIKKAKRLLKKLSPPPKC

SEQ ID NO: 287 - protein, 42 aa, Artificial Sequence
  synthetic polypeptide
HSDAVFTDNYTRLLLKVAAKKYLNSIKKAKRLLKKLSPPPKC

SEQ ID NO: 288 - protein, 42 aa, Artificial Sequence
  synthetic polypeptide
HSDAVFTENYTRLLLKVAAKKYLNSIKKAKRLLKKLSPPPKC

SEQ ID NO: 289 - protein, 42 aa, Artificial Sequence
  synthetic polypeptide
HSDAVFTDNYTRLLAKVAAKKYLNSIKKAKRLLKKLSPPPKC

SEQ ID NO: 290 - protein, 42 aa, Artificial Sequence
  synthetic polypeptide
HSDAVFTDNYTRLFAKVAAKKYLNSIKKAKRLLKKLSPPPKC

SEQ ID NO: 291 - protein, 42 aa, Artificial Sequence
  synthetic polypeptide
HSDAVFTQQYTRLLKQVAAKKYLNSIKKAKRLLKKLSPPPKC

SEQ ID NO: 292 - protein, 42 aa, Artificial Sequence
  synthetic polypeptide
HSDAVFTQQYTRLLAKVALKKYLNSIKKAKRLLKKLSPPPKC

SEQ ID NO: 293 - protein, 42 aa, Artificial Sequence
  synthetic polypeptide
HSDAVFTQQYTRLLAKVALKKYLNSIKKAKELLKKLSPPPKC

SEQ ID NO: 294 - protein, 42 aa, Artificial Sequence
  synthetic polypeptide
HSDAVFTQQYTRLLAKVALKKYLNSIKNAKRLLKKLSPPPKC

SEQ ID NO: 295 - protein, 42 aa, Artificial Sequence; identical to SEQ ID NO: 294
  synthetic polypeptide

SEQ ID NO: 296 - protein, 42 aa, Artificial Sequence
  synthetic polypeptide
HSDAVFTQQYTRLLAKVALKKYLNSIQNAKRLLKKLSPPPKC

SEQ ID NO: 297 - protein, 42 aa, Artificial Sequence
  synthetic polypeptide
HSDAVFTDQYTRLLAKVALKKYLNSIKNAKRLLKKLSPPPKC

SEQ ID NO: 298 - protein, 42 aa, Artificial Sequence; identical to SEQ ID NO: 297
  synthetic polypeptide

SEQ ID NO: 299 - protein, 42 aa, Artificial Sequence
  synthetic polypeptide
HSDAVFTDQYTRLLKQVALKKYLNSIKNAKRLLKKLSPPPKC

SEQ ID NO: 300 - protein, 42 aa, Artificial Sequence
  synthetic polypeptide
HSDAVFTQNYTRLRKQVAAKKYLNSIKKAKRLLKKLSPPPKC

SEQ ID NO: 301 - protein, 42 aa, Artificial Sequence
  synthetic polypeptide
HSDAVFTQQYTRLRKQVAAKKYLNSIKKAKRLLKKLSPPPKC

SEQ ID NO: 302 - protein, 42 aa, Artificial Sequence
  synthetic polypeptide
HSDAVFTDNYTRLRKQVAAKKYLNSIKKAKRLLKKLSPPPKC

SEQ ID NO: 303 - protein, 42 aa, Artificial Sequence
  synthetic polypeptide
HSDAVFTENYTRLRKQVAAKKYLNSIKKAKRLLKKLSPPPKC

SEQ ID NO: 304 - protein, 42 aa, Artificial Sequence
  synthetic polypeptide
HSDAVFTDNYTRLRKQVAAKKYLNSIKKAKELLKKLSPPPKC

SEQ ID NO: 305 - protein, 42 aa, Artificial Sequence
  synthetic polypeptide
HSDAVFTQNYTRLRLQVAAKKYLNSIKKAKELLKKLSPPPKC

SEQ ID NO: 306 - protein, 42 aa, Artificial Sequence
  synthetic polypeptide
HSDAVFTQQYTRLRKQVAAKKYLNSIKKAKELLKKLSPPPKC

SEQ ID NO: 307 - protein, 42 aa, Artificial Sequence
  synthetic polypeptide
HSDAVFTQQYTRLRLQVALKKYLNSIKKAKRLLKKLSPPPKC

SEQ ID NO: 308 - protein, 42 aa, Artificial Sequence
  synthetic polypeptide
HSDAVFTQQYTRLRKQVALKKYLNSIKKAKELLKKLSPPPKC

SEQ ID NO: 309 - protein, 42 aa, Artificial Sequence
  synthetic polypeptide
HSDAVFTQQYTRLRKQVALKKYLNSIKNAKRLLKKLSPPPKC

SEQ ID NO: 310 - protein, 42 aa, Artificial Sequence; identical to SEQ ID NO: 309
  synthetic polypeptide

SEQ ID NO: 311 - protein, 42 aa, Artificial Sequence
  synthetic polypeptide
HSDAVFTQQYTRLRKQVALKKYLNSIQNAKRLLKKLSPPPKC

SEQ ID NO: 312 - protein, 42 aa, Artificial Sequence
  synthetic polypeptide
HSDAVFTDQYTRLRKQVALKKYLNSIKNAKRLLKKLSPPPKC

SEQ ID NO: 313 - protein, 42 aa, Artificial Sequence; identical to SEQ ID NO: 312
  synthetic polypeptide

SEQ ID NO: 314 - protein, 42 aa, Artificial Sequence
  synthetic polypeptide
HSDAVFTDQYTRLRKQVALKKYLNSIKNAKRLLEKLSPPPKC

SEQ ID NO: 315 - protein, 38 aa, Artificial Sequence
  synthetic polypeptide
HSDAVFTDQYTRLLKQVAAKKYLNSIKKAKRLLRKLKC

SEQ ID NO: 316 - protein, 36 aa, Artificial Sequence
  synthetic polypeptide
HSDAVFTDNYTRLRKQVAAKKYLQSIKNSRTSPPPK

SEQ ID NO: 317 - protein, 35 aa, Artificial Sequence
  synthetic polypeptide
HSDAVFTDNYTRLRXQVAAXKYLQSIKNSRTSPPP

SEQ ID NO: 318 - protein, 35 aa, Artificial Sequence
  synthetic polypeptide
HSDAVFTDNYTRLLLKVAAKKYLQSIKNSRTSPPP

SEQ ID NO: 319 - protein, 41 aa, Artificial Sequence; identical to SEQ ID NO: 92
  synthetic polypeptide

SEQ ID NO: 320 - protein, 37 aa, Artificial Sequence; identical to SEQ ID NO: 112
  synthetic polypeptide

SEQ ID NO: 321 - protein, 36 aa, Artificial Sequence; identical to SEQ ID NO: 113
  synthetic polypeptide

SEQ ID NO: 322 - protein, 37 aa, Artificial Sequence; identical to SEQ ID NO: 117
  synthetic polypeptide

SEQ ID NO: 323 - protein, 37 aa, Artificial Sequence; identical to SEQ ID NO: 119
  synthetic polypeptide

SEQ ID NO: 324 - protein, 37 aa, Artificial Sequence; identical to SEQ ID NO: 120
  synthetic polypeptide

SEQ ID NO: 325 - protein, 37 aa, Artificial Sequence; identical to SEQ ID NO: 121
  synthetic polypeptide

SEQ ID NO: 326 - protein, 37 aa, Artificial Sequence; identical to SEQ ID NO: 123
  synthetic polypeptide

SEQ ID NO: 327 - protein, 37 aa, Artificial Sequence; identical to SEQ ID NO: 125
  synthetic polypeptide

SEQ ID NO: 328 - protein, 37 aa, Artificial Sequence; identical to SEQ ID NO: 126
  synthetic polypeptide

SEQ ID NO: 329 - protein, 37 aa, Artificial Sequence; identical to SEQ ID NO: 127
  synthetic polypeptide

SEQ ID NO: 330 - protein, 37 aa, Artificial Sequence; identical to SEQ ID NO: 128
  synthetic polypeptide

SEQ ID NO: 331 - protein, 37 aa, Artificial Sequence; identical to SEQ ID NO: 131
  synthetic polypeptide

SEQ ID NO: 332 - protein, 37 aa, Artificial Sequence; identical to SEQ ID NO: 133
  synthetic polypeptide

SEQ ID NO: 333 - protein, 37 aa, Artificial Sequence; identical to SEQ ID NO: 134
  synthetic polypeptide

SEQ ID NO: 334 - protein, 37 aa, Artificial Sequence; identical to SEQ ID NO: 138
  synthetic polypeptide

SEQ ID NO: 335 - protein, 37 aa, Artificial Sequence; identical to SEQ ID NO: 139
  synthetic polypeptide

SEQ ID NO: 336 - protein, 42 aa, Artificial Sequence; identical to SEQ ID NO: 151
  synthetic polypeptide

SEQ ID NO: 337 - protein, 42 aa, Artificial Sequence; identical to SEQ ID NO: 152
  synthetic polypeptide

SEQ ID NO: 338 - protein, 42 aa, Artificial Sequence
  synthetic polypeptide
HSDAVFTQNYTRLLKQVAAKKVLNSIKKAKRELLRKLSPPPK

SEQ ID NO: 339 - protein, 41 aa, Artificial Sequence
  synthetic polypeptide
HSDAVFTDNYTLKKAKLALQKYLQSIKNKRELLRKLSPPPK

SEQ ID NO: 340 - protein, 41 aa, Artificial Sequence; identical to SEQ ID NO: 160
  synthetic polypeptide

SEQ ID NO: 341 - protein, 41 aa, Artificial Sequence; identical to SEQ ID NO: 161
  synthetic polypeptide

SEQ ID NO: 342 - protein, 41 aa, Artificial Sequence; identical to SEQ ID NO: 164
  synthetic polypeptide

SEQ ID NO: 343 - protein, 41 aa, Artificial Sequence; identical to SEQ ID NO: 170
  synthetic polypeptide

SEQ ID NO: 344 - protein, 41 aa, Artificial Sequence; identical to SEQ ID NO: 172
  synthetic polypeptide

SEQ ID NO: 345 - protein, 41 aa, Artificial Sequence; identical to SEQ ID NO: 173
  synthetic polypeptide

SEQ ID NO: 346 - protein, 41 aa, Artificial Sequence; identical to SEQ ID NO: 174
  synthetic polypeptide

SEQ ID NO: 347 - protein, 41 aa, Artificial Sequence
  synthetic polypeptide
HSDAVFTQQYTRLLAKVALKKVLNSIKKAKELLKKLSPPPK

SEQ ID NO: 348 - protein, 41 aa, Artificial Sequence; identical to SEQ ID NO: 192
  synthetic polypeptide

SEQ ID NO: 349 - protein, 42 aa, Artificial Sequence; identical to SEQ ID NO: 205
  synthetic polypeptide

SEQ ID NO: 350 - protein, 38 aa, Artificial Sequence
  synthetic polypeptide
HSDAVFTDNYTRLLKQVAAKKYLNSIKKAKRLLRKLKC

SEQ ID NO: 351 - protein, 37 aa, Artificial Sequence; identical to SEQ ID NO: 226
  synthetic polypeptide

SEQ ID NO: 352 - protein, 38 aa, Artificial Sequence; identical to SEQ ID NO: 230
  synthetic polypeptide

SEQ ID NO: 353 - protein, 38 aa, Artificial Sequence; identical to SEQ ID NO: 232
  synthetic polypeptide

SEQ ID NO: 354 - protein, 38 aa, Artificial Sequence; identical to SEQ ID NO: 233
  synthetic polypeptide

SEQ ID NO: 355 - protein, 38 aa, Artificial Sequence; identical to SEQ ID NO: 234
  synthetic polypeptide

SEQ ID NO: 356 - protein, 38 aa, Artificial Sequence; identical to SEQ ID NO: 236
  synthetic polypeptide

SEQ ID NO: 357 - protein, 38 aa, Artificial Sequence; identical to SEQ ID NO: 238
  synthetic polypeptide

SEQ ID NO: 358 - protein, 38 aa, Artificial Sequence; identical to SEQ ID NO: 239
  synthetic polypeptide

SEQ ID NO: 359 - protein, 38 aa, Artificial Sequence; identical to SEQ ID NO: 240
  synthetic polypeptide

SEQ ID NO: 360 - protein, 38 aa, Artificial Sequence; identical to SEQ ID NO: 241
  synthetic polypeptide

SEQ ID NO: 361 - protein, 38 aa, Artificial Sequence; identical to SEQ ID NO: 244
  synthetic polypeptide

SEQ ID NO: 362 - protein, 38 aa, Artificial Sequence; identical to SEQ ID NO: 246
  synthetic polypeptide

SEQ ID NO: 363 - protein, 38 aa, Artificial Sequence; identical to SEQ ID NO: 247
  synthetic polypeptide

SEQ ID NO: 364 - protein, 38 aa, Artificial Sequence; identical to SEQ ID NO: 251
  synthetic polypeptide

SEQ ID NO: 365 - protein, 38 aa, Artificial Sequence; identical to SEQ ID NO: 252
  synthetic polypeptide

SEQ ID NO: 366 - protein, 43 aa, Artificial Sequence; identical to SEQ ID NO: 264
  synthetic polypeptide

SEQ ID NO: 367 - protein, 43 aa, Artificial Sequence; identical to SEQ ID NO: 265
  synthetic polypeptide

SEQ ID NO: 368 - protein, 43 aa, Artificial Sequence; identical to SEQ ID NO: 271
  synthetic polypeptide

SEQ ID NO: 369 - protein, 42 aa, Artificial Sequence; identical to SEQ ID NO: 272
  synthetic polypeptide

SEQ ID NO: 370 - protein, 42 aa, Artificial Sequence; identical to SEQ ID NO: 273
  synthetic polypeptide

SEQ ID NO: 371 - protein, 42 aa, Artificial Sequence; identical to SEQ ID NO: 274
  synthetic polypeptide

SEQ ID NO: 372 - protein, 42 aa, Artificial Sequence; identical to SEQ ID NO: 277
  synthetic polypeptide

SEQ ID NO: 373 - protein, 42 aa, Artificial Sequence; identical to SEQ ID NO: 283
  synthetic polypeptide

SEQ ID NO: 374 - protein, 42 aa, Artificial Sequence; identical to SEQ ID NO: 285
  synthetic polypeptide

SEQ ID NO: 375 - protein, 42 aa, Artificial Sequence; identical to SEQ ID NO: 286
  synthetic polypeptide

SEQ ID NO: 376 - protein, 42 aa, Artificial Sequence; identical to SEQ ID NO: 287
  synthetic polypeptide

SEQ ID NO: 377 - protein, 42 aa, Artificial Sequence; identical to SEQ ID NO: 293
  synthetic polypeptide

SEQ ID NO: 378 - protein, 42 aa, Artificial Sequence
  synthetic polypeptide
HSDAVFTQNYTRLRKQVAAKKYLNSIKKAKELLKKLSPPPKC

SEQ ID NO: 379 - protein, 41 aa, Artificial Sequence
  synthetic polypeptide
HSDAVFTDNYTRLLAKLALQKYLQSIKNKYRLLRKLSPPPC

SEQ ID NO: 380 - protein, 37 aa, Artificial Sequence
  synthetic polypeptide
HSDAVFTDNYTRLLKQVAAKKYLNSIKKAKRLLRKLC

SEQ ID NO: 381 - protein, 36 aa, Artificial Sequence
  synthetic polypeptide
HSDAVFTDNYTRLLKQVAAKKYLNSIKNKRLLRKLC

SEQ ID NO: 382 - protein, 37 aa, Artificial Sequence
  synthetic polypeptide
HSDAVFTDNYTRLLKQVAAKKYLNSIKKAKRLLKKLC

SEQ ID NO: 383 - protein, 37 aa, Artificial Sequence
  synthetic polypeptide
HSDAVFTQNYTRLLKQVAAKKYLNSIKKAKRLLKKLC

SEQ ID NO: 384 - protein, 37 aa, Artificial Sequence
  synthetic polypeptide
HSDAVFTQNYTRLLLKVAAKKYLNSIKKAKRLLKKLC

SEQ ID NO: 385 - protein, 37 aa, Artificial Sequence
  synthetic polypeptide
HSDAVFTDNYTRLLLKVAAKKYLNSIKKAKRLLKKLC

SEQ ID NO: 386 - protein, 37 aa, Artificial Sequence
  synthetic polypeptide
HSDAVFTDNYTRLLAKVAAKKYLNSIKKAKRLLKKLC

SEQ ID NO: 387 - protein, 37 aa, Artificial Sequence
  synthetic polypeptide
HSDAVFTQQYTRLLKQVAAKKYLNSIKKAKRLLKKLC

SEQ ID NO: 388 - protein, 37 aa, Artificial Sequence
  synthetic polypeptide
HSDAVFTQQYTRLLAKVALKKYLNSIKKAKRLLKKLC

SEQ ID NO: 389 - protein, 37 aa, Artificial Sequence
  synthetic polypeptide
HSDAVFTQQYTRLLAKVALKKYLNSIKKAKELLKKLC

SEQ ID NO: 390 - protein, 37 aa, Artificial Sequence
  synthetic polypeptide
HSDAVFTQQYTRLLAKVALKKYLNSIKNAKRLLKKLC

SEQ ID NO: 391 - protein, 37 aa, Artificial Sequence
  synthetic polypeptide
HSDAVFTDQYTRLLAKVALKKYLNSIKNAKRLLKKLC

SEQ ID NO: 392 - protein, 37 aa, Artificial Sequence
  synthetic polypeptide
HSDAVFTDQYTRLLKQVALKKVINSIKNAKRLLKKLC

SEQ ID NO: 393 - protein, 37 aa, Artificial Sequence
  synthetic polypeptide
HSDAVFTQNYTRLRKQVAAKKYLNSIKKAKRLLKKLC

SEQ ID NO: 394 - protein, 37 aa, Artificial Sequence
  synthetic polypeptide
HSDAVFTDNYTRLRKQVAAKKYLNSIKKAKELLKKLC

SEQ ID NO: 395 - protein, 37 aa, Artificial Sequence
  synthetic polypeptide
HSDAVFTQNYTRLRKQVAAKKYLNSIKKAKELLKKLC

SEQ ID NO: 396 - protein, 42 aa, Artificial Sequence
  synthetic polypeptide
HSDAVFTDQYTRLLKQVAAKKYLQWIKKAKRELLRKLSPPPC

SEQ ID NO: 397 - protein, 42 aa, Artificial Sequence
  synthetic polypeptide
HSDAVFTQQYTRLLKQVAAKKYLQWIKKAKRELLRKLSPPPC

SEQ ID NO: 398 - protein, 42 aa, Artificial Sequence
  synthetic polypeptide
HSDAVFTQNYTRLLKQVAAKKYLNSIKKAKRELLRKLSPPPC

SEQ ID NO: 399 - protein, 41 aa, Artificial Sequence
  synthetic polypeptide
HSDAVFTDNYTKLLAKLALQKYLQSIKNKRELLRKLSPPPC

SEQ ID NO: 400 - protein, 41 aa, Artificial Sequence
  synthetic polypeptide
HSDAVFTQNYTKLLAKLALQKYLQSIKNKRELLRKLSPPPC

SEQ ID NO: 401 - protein, 41 aa, Artificial Sequence
  synthetic polypeptide
HSDAVFTQNYTKLLAKLALQKYLQSIKNKRQLLRKLSPPPC

SEQ ID NO: 402 - protein, 41 aa, Artificial Sequence
  synthetic polypeptide
HSDAVFTQNYTKLLAKLALQKYLQSIKNKARLLRKLSPPPC

SEQ ID NO: 403 - protein, 41 aa, Artificial Sequence
  synthetic polypeptide
HSDAVFTDNYTRLLKQVAAKKYLNSIKKAKRLLKKLSPPPC

SEQ ID NO: 404 - protein, 41 aa, Artificial Sequence
  synthetic polypeptide
HSDAVFTQNYTRLLKQVAAKKYLNSIKKAKRLLKKLSPPPC

SEQ ID NO: 405 - protein, 41 aa, Artificial Sequence
  synthetic polypeptide
HSDAVFTQNYTRLLLKVAAKKYLNSIKKAKRLLKKLSPPPC

SEQ ID NO: 406 - protein, 41 aa, Artificial Sequence
  synthetic polypeptide
HSDAVFTDNYTRLLLKVAAKKYLNSIKKAKRLLKKLSPPPC

SEQ ID NO: 407 - protein, 41 aa, Artificial Sequence
  synthetic polypeptide
HSDAVFTQQYTRLLAKVALKKYLNSIKKAKELLKKLSPPPC

SEQ ID NO: 408 - protein, 41 aa, Artificial Sequence
  synthetic polypeptide
HSDAVFTQNYTRLRKQVAAKKYLNSIKKAKELLKKLSPPPC

SEQ ID NO: 409 - protein, 10 aa, Artificial Sequence
  synthetic peptide
XXXXXXXXXX

SEQ ID NO: 410 - protein, 14 aa, Artificial Sequence
  synthetic peptide
XXXXXXXXXXXXXX

SEQ ID NO: 411 - protein, 18 aa, Artificial Sequence
  synthetic peptide
XXXXXXXXXXXXXXXXXX

SEQ ID NO: 412 - protein, 22 aa, Artificial Sequence
  synthetic peptide
XXXXXXXXXXXXXXXXXXXXXX

SEQ ID NO: 413 - protein, 5 aa, Artificial Sequence
  synthetic peptide
LKPPP

SEQ ID NO: 414 - protein, 5 aa, Artificial Sequence
  synthetic peptide
XXXXX

SEQ ID NO: 415 - protein, 9 aa, Artificial Sequence
  synthetic peptide
XXXXXXXXX

SEQ ID NO: 416 - protein, 13 aa, Artificial Sequence
  synthetic peptide
XXXXXXXXXXXXX

SEQ ID NO: 417 - protein, 17 aa, Artificial Sequence
  synthetic peptide
XXXXXXXXXXXXXXXXX

SEQ ID NO: 418 - protein, 21 aa, Artificial Sequence
  synthetic peptide
XXXXXXXXXXXXXXXXXXXXX

SEQ ID NO: 419 - protein, 6 aa, Artificial Sequence
  synthetic peptide
XXXXXX

SEQ ID NO: 420 - protein, 10 aa, Artificial Sequence
  synthetic peptide
XXXXXXXXXX

SEQ ID NO: 421 - protein, 14 aa, Artificial Sequence
  synthetic peptide
XXXXXXXXXXXXXX

SEQ ID NO: 422 - protein, 18 aa, Artificial Sequence
  synthetic peptide
XXXXXXXXXXXXXXXXXX

SEQ ID NO: 423 - protein, 22 aa, Artificial Sequence
  synthetic peptide
XXXXXXXXXXXXXXXXXXXXXX

SEQ ID NO: 424 - protein, 40 aa, Artificial Sequence
  synthetic peptide
XXXXXXTXXXTXXXXXXAXXXXXXXXXXXXXXXXXXXXXX

SEQ ID NO: 425 - protein, 38 aa, Artificial Sequence; identical to SEQ ID NO: 253
  synthetic polypeptide

SEQ ID NO: 426 - protein, 38 aa, Artificial Sequence; identical to SEQ ID NO: 253
  synthetic polypeptide

SEQ ID NO: 427 - protein, 38 aa, Artificial Sequence
  synthetic polypeptide
HSDAVFTQQYTRLRKQVAAKKYLNSIKKAKELLKKLKK

SEQ ID NO: 428 - protein, 37 aa, Artificial Sequence; identical to SEQ ID NO: 140
  synthetic polypeptide

SEQ ID NO: 429 - protein, 37 aa, Artificial Sequence; identical to SEQ ID NO: 140
  synthetic polypeptide

SEQ ID NO: 430 - protein, 37 aa, Artificial Sequence
  synthetic polypeptide
HSDAVFTQQYTRLRKQVAAQKYLNSIKKAKELLKKLK

SEQ ID NO: 431 - protein, 37 aa, Artificial Sequence
  synthetic polypeptide
HSDAVFTQQYTRLRKQVAAQKYLQSIKKAKELLKKLK

SEQ ID NO: 432 - protein, 37 aa, Artificial Sequence
  synthetic polypeptide
HSDAVFTQQYTRLRKQVAAQKYLQSIKQAKELLKKLK

SEQ ID NO: 433 - protein, 37 aa, Artificial Sequence; identical to SEQ ID NO: 432
  synthetic polypeptide

SEQ ID NO: 434 - protein, 38 aa, Artificial Sequence
  synthetic polypeptide
HSDAVFTQQYTRLRKQVAAQKYLQSIKQAKELLKKLKC

SEQ ID NO: 435 - protein, 37 aa, Artificial Sequence; identical to SEQ ID NO: 431
  synthetic polypeptide

SEQ ID NO: 436 - protein, 38 aa, Artificial Sequence
  synthetic polypeptide
HSDAVFTQQYTRLRKQVAAQKYLQSIKKAKELLKKLKC

SEQ ID NO: 437 - protein, 38 aa, Artificial Sequence
  synthetic polypeptide
HSDAVFTQQYTRLRRQVAAQRYLQSIRRARELLRRLKC

SEQ ID NO: 438 - protein, 37 aa, Artificial Sequence
  synthetic polypeptide
HSDAVFTQQYTRLRRQVAAQRYLQSIRRARELLRRLK

SEQ ID NO: 439 - protein, 38 aa, Artificial Sequence
  synthetic polypeptide
HSDAVFTQQYTRLRRQVAAQRYLQSIRRARELLRRLKK

SEQ ID NO: 440 - protein, 38 aa, Artificial Sequence; identical to SEQ ID NO: 439
  synthetic polypeptide

SEQ ID NO: 441 - protein, 37 aa, Artificial Sequence; identical to SEQ ID NO: 438
  synthetic polypeptide

SEQ ID NO: 442 - protein, 37 aa, Artificial Sequence; identical to SEQ ID NO: 438
  synthetic polypeptide

SEQ ID NO: 443 - protein, 38 aa, Artificial Sequence
  synthetic polypeptide
HSDAVFTQQYTRLLAKLALQKYLQSIKQKRYELLKKLK

SEQ ID NO: 444 - protein, 38 aa, Artificial Sequence
  synthetic polypeptide
HSDAVFTQQYTRLLLKVAAQKYLQSIKQKRYELLKKLK

SEQ ID NO: 445 - protein, 37 aa, Artificial Sequence
  synthetic polypeptide
HSDAVFTQQYTRLLLKVAAQKYLQSIKQKRELLKKLK

SEQ ID NO: 446 - protein, 37 aa, Artificial Sequence
  synthetic polypeptide
HSDAVFTQQYTRLLLKVAAQKYLQSIKNKRELLKKLK

SEQ ID NO: 447 - protein, 38 aa, Artificial Sequence
  synthetic polypeptide
HSDAVFTQQYTRLLLKVAAKKYLQSIKQKRYELLKKLK

SEQ ID NO: 448 - protein, 37 aa, Artificial Sequence
  synthetic polypeptide
HSDAVFTQQYTRLLLKVAAKKYLQSIKQKRELLKKLK

SEQ ID NO: 449 - protein, 37 aa, Artificial Sequence
  synthetic polypeptide
HSDAVFTQQYTRLLLKVAAQKYLQSIKKAKELLKKLK

SEQ ID NO: 450 - protein, 38 aa, Artificial Sequence
  synthetic polypeptide
HSDAVFTQQYTRLLLKVAAQKYLQSIKQSRYELLKKLK

SEQ ID NO: 451 - protein, 37 aa, Artificial Sequence
  synthetic polypeptide
HSDAVFTQQYTRLLLKVAAQKYLQSIKQSRELLKKLK

SEQ ID NO: 452 - protein, 42 aa, Artificial Sequence
  synthetic polypeptide
HSDAVFTQQYTRLLLKVAAQKYLQSIKQSRYELLKKLSPPPK

SEQ ID NO: 453 - protein, 41 aa, Artificial Sequence
  synthetic polypeptide
HSDAVFTQQYTRLLLKVAAQKYLQSIKQSRELLKKLSPPPK

SEQ ID NO: 454 - protein, 41 aa, Artificial Sequence
  synthetic polypeptide
HSDAVFTQQYTRLLLKVAAQKYLQSIKQKRELLKKLSPPPK

SEQ ID NO: 455 - protein, 38 aa, Artificial Sequence; identical to SEQ ID NO: 444
  synthetic polypeptide

SEQ ID NO: 456 - protein, 38 aa, Artificial Sequence
  synthetic polypeptide
HSDAVFTQQYTRLLARLALQRYLQSIRQRRYELLRRLK

SEQ ID NO: 457 - protein, 39 aa, Artificial Sequence
  synthetic polypeptide
HSDAVFTQQYTRLLARLALQRYLQSIRQRRYELLRRLKC

SEQ ID NO: 458 - protein, 39 aa, Artificial Sequence; identical to SEQ ID NO: 457
  synthetic polypeptide

SEQ ID NO: 459 - protein, 39 aa, Artificial Sequence
  synthetic polypeptide
HSDAVFTQQYTRLLARLALQRYLQSIRQRRYELLRRLKK

SEQ ID NO: 460 - protein, 39 aa, Artificial Sequence; identical to SEQ ID NO: 459
  synthetic polypeptide

SEQ ID NO: 461 - protein, 37 aa, Artificial Sequence
  synthetic polypeptide
HSDAVFTQQYTRLRAQVAARRYLQSIRQSRELLRRLK

SEQ ID NO: 462 - protein, 37 aa, Artificial Sequence
  synthetic polypeptide
HSDAVFTQQYTRLLRQVAARRYLQSIRQSRELLRRLK

SEQ ID NO: 463 - protein, 37 aa, Artificial Sequence
  synthetic polypeptide
HSDAVFTQQYTRLRAQVAAQRYLQSIRQSRELLRRLK

SEQ ID NO: 464 - protein, 38 aa, Artificial Sequence; identical to SEQ ID NO: 443
  synthetic polypeptide

SEQ ID NO: 465 - protein, 38 aa, Artificial Sequence; identical to SEQ ID NO: 444
  synthetic polypeptide

SEQ ID NO: 466 - protein, 37 aa, Artificial Sequence; identical to SEQ ID NO: 445
  synthetic polypeptide

SEQ ID NO: 467 - protein, 37 aa, Artificial Sequence; identical to SEQ ID NO: 446
  synthetic polypeptide

SEQ ID NO: 468 - protein, 38 aa, Artificial Sequence; identical to SEQ ID NO: 447
  synthetic polypeptide

SEQ ID NO: 469 - protein, 37 aa, Artificial Sequence; identical to SEQ ID NO: 448
  synthetic polypeptide

SEQ ID NO: 470 - protein, 37 aa, Artificial Sequence; identical to SEQ ID NO: 449
  synthetic polypeptide

SEQ ID NO: 471 - protein, 38 aa, Artificial Sequence; identical to SEQ ID NO: 450
  synthetic polypeptide

SEQ ID NO: 472 - protein, 37 aa, Artificial Sequence; identical to SEQ ID NO: 451
  synthetic polypeptide

SEQ ID NO: 473 - protein, 42 aa, Artificial Sequence; identical to SEQ ID NO: 452
  synthetic polypeptide

SEQ ID NO: 474 - protein, 41 aa, Artificial Sequence; identical to SEQ ID NO: 453
  synthetic polypeptide

SEQ ID NO: 475 - protein, 41 aa, Artificial Sequence; identical to SEQ ID NO: 454
  synthetic polypeptide

SEQ ID NO: 476 - protein, 38 aa, Artificial Sequence
  synthetic polypeptide
HSDAVFTQQYTRLLLKVAVQKYLQSIKQKRYELLKKLK

SEQ ID NO: 477 - protein, 38 aa, Artificial Sequence; identical to SEQ ID NO: 456
  synthetic polypeptide

SEQ ID NO: 478 - protein, 39 aa, Artificial Sequence; identical to SEQ ID NO: 457
  synthetic polypeptide

SEQ ID NO: 479 - protein, 39 aa, Artificial Sequence; identical to SEQ ID NO: 459
  synthetic polypeptide

SEQ ID NO: 480 - protein, 37 aa, Artificial Sequence; identical to SEQ ID NO: 461
  synthetic polypeptide

SEQ ID NO: 481 - protein, 37 aa, Artificial Sequence; identical to SEQ ID NO: 462
  synthetic polypeptide

SEQ ID NO: 482 - protein, 37 aa, Artificial Sequence; identical to SEQ ID NO: 463
  synthetic polypeptide

SEQ ID NO: 483 - protein, 39 aa, Artificial Sequence
  synthetic polypeptide
HSDAVFTQQYTRLLAKLALQKYLQSIKQKRYELLKKLKC

SEQ ID NO: 484 - protein, 39 aa, Artificial Sequence
  synthetic polypeptide
HSDAVFTQQYTRLLLKVAAQKYLQSIKQKRYELLKKLKC

SEQ ID NO: 485 - protein, 38 aa, Artificial Sequence
  synthetic polypeptide
HSDAVFTQQYTRLLLKVAAQKYLQSIKQKRELLKKLKC

SEQ ID NO: 486 - protein, 38 aa, Artificial Sequence
  synthetic polypeptide
HSDAVFTQQYTRLLLKVAAQKYLQSIKNKRELLKKLKC

SEQ ID NO: 487 - protein, 39 aa, Artificial Sequence
  synthetic polypeptide
HSDAVFTQQYTRLLLKVAAKKYLQSIKQKRYELLKKLKC

SEQ ID NO: 488 - protein, 38 aa, Artificial Sequence
  synthetic polypeptide
HSDAVFTQQYTRLLLKVAAKKYLQSIKQKRELLKKLKC

SEQ ID NO: 489 - protein, 38 aa, Artificial Sequence
  synthetic polypeptide
HSDAVFTQQYTRLLLKVAAQKYLQSIKKAKELLKKLKC

SEQ ID NO: 490 - protein, 39 aa, Artificial Sequence
  synthetic polypeptide
HSDAVFTQQYTRLLLKVAAQKYLQSIKQSRYELLKKLKC

SEQ ID NO: 491 - protein, 38 aa, Artificial Sequence
  synthetic polypeptide
HSDAVFTQQYTRLLLKVAAQKYLQSIKQSRELLKKLKC

SEQ ID NO: 492 - protein, 43 aa, Artificial Sequence
  synthetic polypeptide
HSDAVFTQQYTRLLLKVAAQKYLQSIKQSRYELLKKLSPPPKC

SEQ ID NO: 493 - protein, 42 aa, Artificial Sequence
  synthetic polypeptide
HSDAVFTQQYTRLLLKVAAQKYLQSIKQSRELLKKLSPPPKC

SEQ ID NO: 494 - protein, 42 aa, Artificial Sequence
  synthetic polypeptide
HSDAVFTQQYTRLLLKVAAQKYLQSIKQKRELLKKLSPPPKC

SEQ ID NO: 495 - protein, 38 aa, Artificial Sequence
  synthetic polypeptide
HSDAVFTQQYTRLRAQVAARRYLQSIRQSRELLRRLKC

SEQ ID NO: 496 - protein, 38 aa, Artificial Sequence; identical to SEQ ID NO: 495
  synthetic polypeptide

SEQ ID NO: 497 - protein, 38 aa, Artificial Sequence
  synthetic polypeptide
HSDAVFTQQYTRLRAQVAAQRYLQSIRQSRELLRRLKC

SEQ ID NO: 498 - protein, 38 aa, Artificial Sequence; identical to SEQ ID NO: 443
  synthetic polypeptide

SEQ ID NO: 499 - protein, 38 aa, Artificial Sequence; identical to SEQ ID NO: 444
  synthetic polypeptide

SEQ ID NO: 500 - protein, 37 aa, Artificial Sequence; identical to SEQ ID NO: 445
  synthetic polypeptide

SEQ ID NO: 501 - protein, 37 aa, Artificial Sequence; identical to SEQ ID NO: 446
  synthetic polypeptide

SEQ ID NO: 502 - protein, 38 aa, Artificial Sequence; identical to SEQ ID NO: 447
  synthetic polypeptide

SEQ ID NO: 503 - protein, 37 aa, Artificial Sequence; identical to SEQ ID NO: 448
  synthetic polypeptide

SEQ ID NO: 504 - protein, 37 aa, Artificial Sequence; identical to SEQ ID NO: 449
  synthetic polypeptide

SEQ ID NO: 505 - protein, 38 aa, Artificial Sequence; identical to SEQ ID NO: 450
  synthetic polypeptide

SEQ ID NO: 506 - protein, 37 aa, Artificial Sequence; identical to SEQ ID NO: 451
  synthetic polypeptide

SEQ ID NO: 507 - protein, 42 aa, Artificial Sequence; identical to SEQ ID NO: 452
  synthetic polypeptide

SEQ ID NO: 508 - protein, 41 aa, Artificial Sequence; identical to SEQ ID NO: 453
  synthetic polypeptide

SEQ ID NO: 509 - protein, 41 aa, Artificial Sequence; identical to SEQ ID NO: 454
  synthetic polypeptide

SEQ ID NO: 510 - protein, 37 aa, Artificial Sequence; identical to SEQ ID NO: 431
  synthetic polypeptide

SEQ ID NO: 511 - protein, 37 aa, Artificial Sequence; identical to SEQ ID NO: 432
  synthetic polypeptide

SEQ ID NO: 512 - protein, 37 aa, Artificial Sequence; identical to SEQ ID NO: 432
  synthetic polypeptide

SEQ ID NO: 513 - protein, 37 aa, Artificial Sequence; identical to SEQ ID NO: 432
  synthetic polypeptide

SEQ ID NO: 514 - protein, 37 aa, Artificial Sequence; identical to SEQ ID NO: 431
  synthetic polypeptide

SEQ ID NO: 515 - protein, 37 aa, Artificial Sequence; identical to SEQ ID NO: 431
  synthetic polypeptide

SEQ ID NO: 516 - protein, 37 aa, Artificial Sequence; identical to SEQ ID NO: 438
  synthetic polypeptide

SEQ ID NO: 517 - protein, 37 aa, Artificial Sequence; identical to SEQ ID NO: 438
  synthetic polypeptide

SEQ ID NO: 518 - protein, 37 aa, Artificial Sequence; identical to SEQ ID NO: 438
  synthetic polypeptide

SEQ ID NO: 519 - protein, 37 aa, Artificial Sequence; identical to SEQ ID NO: 438
  synthetic polypeptide

SEQ ID NO: 520 - protein, 37 aa, Artificial Sequence
  synthetic polypeptide
HSDAVFTQQYTRLRRQVAAQRYLQSIRQARELLRRLK

SEQ ID NO: 521 - protein, 37 aa, Artificial Sequence
  synthetic polypeptide
HSDAVFTQQYTRLRRQVAAQRYLQSIRQSRELLRRLK

SEQ ID NO: 522 - protein, 37 aa, Artificial Sequence; identical to SEQ ID NO: 461
  synthetic polypeptide

SEQ ID NO: 523 - protein, 37 aa, Artificial Sequence; identical to SEQ ID NO: 462
  synthetic polypeptide

SEQ ID NO: 524 - protein, 37 aa, Artificial Sequence; identical to SEQ ID NO: 463
  synthetic polypeptide

SEQ ID NO: 525 - protein, 39 aa, Artificial Sequence
  synthetic polypeptide
HSDAVFTQQYTRLLAKLALQKYLQSIKQKRYELLKKLKK

SEQ ID NO: 526 - protein, 39 aa, Artificial Sequence
  synthetic polypeptide
HSDAVFTQQYTRLLLKVAAQKYLQSIKQKRYELLKKLKK

SEQ ID NO: 527 - protein, 38 aa, Artificial Sequence
  synthetic polypeptide
HSDAVFTQQYTRLLLKVAAQKYLQSIKQKRELLKKLKK

SEQ ID NO: 528 - protein, 38 aa, Artificial Sequence
  synthetic polypeptide
HSDAVFTQQYTRLLLKVAAQKYLQSIKNKRELLKKLKK

SEQ ID NO: 529 - protein, 39 aa, Artificial Sequence
  synthetic polypeptide
HSDAVFTQQYTRLLLKVAAKKYLQSIKQKRYELLKKLKK

SEQ ID NO: 530 - protein, 38 aa, Artificial Sequence
  synthetic polypeptide
HSDAVFTQQYTRLLLKVAAKKYLQSIKQKRELLKKLKK

SEQ ID NO: 531 - protein, 38 aa, Artificial Sequence
  synthetic polypeptide
HSDAVFTQQYTRLLLKVAAQKYLQSIKKAKELLKKLKK

SEQ ID NO: 532 - protein, 39 aa, Artificial Sequence
  synthetic polypeptide
HSDAVFTQQYTRLLLKVAAQKYLQSIKQSRYELLKKLKK

SEQ ID NO: 533 - protein, 38 aa, Artificial Sequence
  synthetic polypeptide
HSDAVFTQQYTRLLLKVAAQKYLQSIKQSRELLKKLKK

SEQ ID NO: 534 - protein, 43 aa, Artificial Sequence
  synthetic polypeptide
HSDAVFTQQYTRLLLKVAAQKYLQSIKQSRYELLKKLSPPPKK

SEQ ID NO: 535 - protein, 42 aa, Artificial Sequence
  synthetic polypeptide
HSDAVFTQQYTRLLLKVAAQKYLQSIKQSRELLKKLSPPPKK

SEQ ID NO: 536 - protein, 42 aa, Artificial Sequence
  synthetic polypeptide
HSDAVFTQQYTRLLLKVAAQKYLQSIKQKRELLKKLSPPPKK

SEQ ID NO: 537 - protein, 38 aa, Artificial Sequence
  synthetic polypeptide
HSDAVFTQQYTRLRKQVAAQKYLQSIKKAKELLKKLKK

SEQ ID NO: 538 - protein, 38 aa, Artificial Sequence
  synthetic polypeptide
HSDAVFTQQYTRLRKQVAAQKYLQSIKQAKELLKKLKK

SEQ ID NO: 539 - protein, 38 aa, Artificial Sequence; identical to SEQ ID NO: 538
  synthetic polypeptide

SEQ ID NO: 540 - protein, 38 aa, Artificial Sequence; identical to SEQ ID NO: 538
  synthetic polypeptide

SEQ ID NO: 541 - protein, 38 aa, Artificial Sequence; identical to SEQ ID NO: 537
  synthetic polypeptide

SEQ ID NO: 542 - protein, 38 aa, Artificial Sequence; identical to SEQ ID NO: 537
  synthetic polypeptide

SEQ ID NO: 543 - protein, 38 aa, Artificial Sequence; identical to SEQ ID NO: 439
  synthetic polypeptide

SEQ ID NO: 544 - protein, 38 aa, Artificial Sequence; identical to SEQ ID NO: 439
  synthetic polypeptide

SEQ ID NO: 545 - protein, 38 aa, Artificial Sequence; identical to SEQ ID NO: 439
  synthetic polypeptide

SEQ ID NO: 546 - protein, 38 aa, Artificial Sequence; identical to SEQ ID NO: 439
  synthetic polypeptide

SEQ ID NO: 547 - protein, 38 aa, Artificial Sequence
  synthetic polypeptide
HSDAVFTQQYTRLRRQVAAQRYLQSIRQARELLRRLKK

SEQ ID NO: 548 - protein, 38 aa, Artificial Sequence
  synthetic polypeptide
HSDAVFTQQYTRLRRQVAAQRYLQSIRQSRELLRRLKK

SEQ ID NO: 549 - protein, 38 aa, Artificial Sequence
  synthetic polypeptide
HSDAVFTQQYTRLRAQVAARRYLQSIRQSRELLRRLKK

SEQ ID NO: 550 - protein, 38 aa, Artificial Sequence
  synthetic polypeptide
HSDAVFTQQYTRLLRQVAARRYLQSIRQSRELLRRLKK

SEQ ID NO: 551 - protein, 38 aa, Artificial Sequence
  synthetic polypeptide
HSDAVFTQQYTRLRAQVAAQRYLQSIRQSRELLRRLKK

SEQ ID NO: 552 - protein, 38 aa, Artificial Sequence; identical to SEQ ID NO: 103
  synthetic polypeptide

SEQ ID NO: 553 - protein, 38 aa, Artificial Sequence; identical to SEQ ID NO: 105
  synthetic polypeptide

SEQ ID NO: 554 - protein, 37 aa, Artificial Sequence; identical to SEQ ID NO: 123
  synthetic polypeptide

SEQ ID NO: 555 - protein, 37 aa, Artificial Sequence; identical to SEQ ID NO: 126
  synthetic polypeptide

SEQ ID NO: 556 - protein, 37 aa, Artificial Sequence; identical to SEQ ID NO: 127
  synthetic polypeptide

SEQ ID NO: 557 - protein, 37 aa, Artificial Sequence; identical to SEQ ID NO: 128
  synthetic polypeptide

SEQ ID NO: 558 - protein, 37 aa, Artificial Sequence; identical to SEQ ID NO: 131
  synthetic polypeptide

SEQ ID NO: 559 - protein, 37 aa, Artificial Sequence; identical to SEQ ID NO: 140
  synthetic polypeptide

SEQ ID NO: 560 - protein, 41 aa, Artificial Sequence; identical to SEQ ID NO: 176
  synthetic polypeptide

SEQ ID NO: 561 - protein, 41 aa, Artificial Sequence; identical to SEQ ID NO: 179
  synthetic polypeptide

SEQ ID NO: 562 - protein, 38 aa, Artificial Sequence; identical to SEQ ID NO: 253
  synthetic polypeptide

SEQ ID NO: 563 - protein, 38 aa, Artificial Sequence; identical to SEQ ID NO: 253
  synthetic polypeptide

SEQ ID NO: 564 - protein, 38 aa, Artificial Sequence; identical to SEQ ID NO: 253
  synthetic polypeptide

SEQ ID NO: 565 - protein, 38 aa, Artificial Sequence; identical to SEQ ID NO: 427
  synthetic polypeptide

SEQ ID NO: 566 - protein, 38 aa, Artificial Sequence; identical to SEQ ID NO: 443
  synthetic polypeptide

SEQ ID NO: 567 - protein, 38 aa, Artificial Sequence; identical to SEQ ID NO: 444
  synthetic polypeptide

SEQ ID NO: 568 - protein, 37 aa, Artificial Sequence; identical to SEQ ID NO: 445
  synthetic polypeptide

SEQ ID NO: 569 - protein, 37 aa, Artificial Sequence; identical to SEQ ID NO: 446
  synthetic polypeptide

SEQ ID NO: 570 - protein, 38 aa, Artificial Sequence; identical to SEQ ID NO: 447
  synthetic polypeptide

SEQ ID NO: 571 - protein, 37 aa, Artificial Sequence; identical to SEQ ID NO: 448
  synthetic polypeptide

SEQ ID NO: 572 - protein, 37 aa, Artificial Sequence; identical to SEQ ID NO: 449
  synthetic polypeptide

SEQ ID NO: 573 - protein, 38 aa, Artificial Sequence; identical to SEQ ID NO: 450
  synthetic polypeptide

SEQ ID NO: 574 - protein, 37 aa, Artificial Sequence; identical to SEQ ID NO: 451
  synthetic polypeptide

SEQ ID NO: 575 - protein, 42 aa, Artificial Sequence; identical to SEQ ID NO: 452
  synthetic polypeptide

SEQ ID NO: 576 - protein, 41 aa, Artificial Sequence; identical to SEQ ID NO: 453
  synthetic polypeptide

SEQ ID NO: 577 - protein, 41 aa, Artificial Sequence; identical to SEQ ID NO: 454
  synthetic polypeptide

SEQ ID NO: 578 - protein, 38 aa, Artificial Sequence; identical to SEQ ID NO: 253
  synthetic polypeptide

SEQ ID NO: 579 - protein, 38 aa, Artificial Sequence; identical to SEQ ID NO: 427
  synthetic polypeptide

SEQ ID NO: 580 - protein, 38 aa, Artificial Sequence; identical to SEQ ID NO: 443
  synthetic polypeptide

SEQ ID NO: 581 - protein, 38 aa, Artificial Sequence; identical to SEQ ID NO: 444
  synthetic polypeptide

SEQ ID NO: 582 - protein, 37 aa, Artificial Sequence; identical to SEQ ID NO: 445
  synthetic polypeptide

SEQ ID NO: 583 - protein, 37 aa, Artificial Sequence; identical to SEQ ID NO: 446
  synthetic polypeptide

SEQ ID NO: 584 - protein, 38 aa, Artificial Sequence; identical to SEQ ID NO: 447
  synthetic polypeptide

SEQ ID NO: 585 - protein, 37 aa, Artificial Sequence; identical to SEQ ID NO: 448
  synthetic polypeptide

SEQ ID NO: 586 - protein, 37 aa, Artificial Sequence; identical to SEQ ID NO: 449
  synthetic polypeptide

SEQ ID NO: 587 - protein, 38 aa, Artificial Sequence; identical to SEQ ID NO: 450
  synthetic polypeptide

SEQ ID NO: 588 - protein, 37 aa, Artificial Sequence; identical to SEQ ID NO: 451
  synthetic polypeptide

SEQ ID NO: 589 - protein, 42 aa, Artificial Sequence; identical to SEQ ID NO: 452
  synthetic polypeptide

SEQ ID NO: 590 - protein, 41 aa, Artificial Sequence; identical to SEQ ID NO: 453
  synthetic polypeptide

SEQ ID NO: 591 - protein, 41 aa, Artificial Sequence; identical to SEQ ID NO: 454
  synthetic polypeptide

SEQ ID NO: 592 - protein, 38 aa, Artificial Sequence
  synthetic polypeptide
HSDAVFTQQYTRLRRQVAAKRYLQSIRRARELLRRLKC

SEQ ID NO: 593 - protein, 38 aa, Artificial Sequence
  synthetic polypeptide
HSDAVFTQQYTRLRRQVAAKRYLQSIRRARELLRRLKK

SEQ ID NO: 594 - protein, 38 aa, Artificial Sequence; identical to SEQ ID NO: 456
  synthetic polypeptide

SEQ ID NO: 595 - protein, 38 aa, Artificial Sequence
  synthetic polypeptide
HSDAVFTQQYTRLLLRVAAQRYLQSIRQRRYELLRRLK

SEQ ID NO: 596 - protein, 37 aa, Artificial Sequence
  synthetic polypeptide
HSDAVFTQQYTRLLLRVAAQRYLQSIRQRRELLRRLK

SEQ ID NO: 597 - protein, 37 aa, Artificial Sequence
  synthetic polypeptide
HSDAVFTQQYTRLLLRVAAQRYLQSIRNRRELLRRLK

SEQ ID NO: 598 - protein, 38 aa, Artificial Sequence
  synthetic polypeptide
HSDAVFTQQYTRLRKQVAAKKYLQSIKKAKELLKKLKC

SEQ ID NO: 599 - protein, 37 aa, Artificial Sequence
  synthetic polypeptide
HSDAVFTQQYTRLRKQVAAKKYLQSIKKAKELLKKLK

SEQ ID NO: 600 - protein, 37 aa, Artificial Sequence
  synthetic polypeptide
HSDAVFTQQYTRLRRQVAARRYLQSIRRARELLRRLK

SEQ ID NO: 601 - protein, 37 aa, Artificial Sequence; identical to SEQ ID NO: 140
  synthetic polypeptide

SEQ ID NO: 602 - protein, 37 aa, Artificial Sequence; identical to SEQ ID NO: 140
  synthetic polypeptide

SEQ ID NO: 603 - protein, 37 aa, Artificial Sequence; identical to SEQ ID NO: 140
  synthetic polypeptide

SEQ ID NO: 604 - protein, 38 aa, Artificial Sequence; identical to SEQ ID NO: 253
  synthetic polypeptide

SEQ ID NO: 605 - protein, 38 aa, Artificial Sequence; identical to SEQ ID NO: 253
  synthetic polypeptide